(19) United States
(12) Patent Application Publication
(10) Pub. No.: US 2025/0057840 A1
(43) Pub. Date: Feb. 20, 2025

(54) COMPOSITIONS AND METHODS FOR THE TREATMENT OF DISEASES WITH ISOCITRATE DEHYDROGENASE 1/2 MUTATIONS

(71) Applicant: Fred Hutchison Cancer Center, Seattle, WA (US)

(72) Inventor: Sita Kugel, Seattle, WA (US)

(73) Assignee: Fred Hutchinson Cancer Center, Seattle, WA (US)

(21) Appl. No.: 18/805,988

(22) Filed: Aug. 15, 2024

Related U.S. Application Data
(60) Provisional application No. 63/519,717, filed on Aug. 15, 2023.

(51) Int. Cl. A61K 31/506; A61K 31/517; A61P 35/00; G01N 33/574

(52) U.S. Cl. CPC A61K 31/506; A61K 31/517; A61P 35/00; G01N 33/57484; G01N 2440/14

(57) ABSTRACT

Compositions and methods for treatment of conditions associated with IDH1/2 mutation(s) are disclosed. The compositions and methods include administering an SRC inhibitor and a p70 S6 kinase/AKT (S6K/AKT) inhibitor. Examples of conditions associated with IDH1/2 mutation(s) include intrahepatic cholangiocarcinoma (ICC), oligodendrogliomas, astrocytomas, glioblastomas, leukemias, adenocarcinoma, gliomas, melanomas, oligoastrocytomas, invasive breast carcinoma, invasive ductal carcinoma, and myelodysplastic syndromes.

### CROSS-REFERENCE TO RELATED APPLICATION

This application claims priority to U.S. Provisional Patent Application No. 63/519,717 filed on Aug. 15, 2023, which is incorporated herein by reference in its entirety as if fully set forth herein.

### STATEMENT REGARDING FEDERALLY SPONSORED RESEARCH OR DEVELOPMENT

This invention was made with government support under CA255015 awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is F053-190US-Seq.xml. The file is 16,806 bytes, was created Oct. 25, 2024, and is being submitted electronically via Patent Center

### FIELD OF THE DISCLOSURE

The current disclosure provides compositions and methods for treating conditions associated with Isocitrate Dehydrogenase 1 and/or 2 (IDH1/2) mutation(s). The compositions and methods include administering an SRC inhibitor and a p70 S6 kinase/AKT (S6K/AKT) inhibitor. Examples of conditions associated with IDH1/2 mutation(s) include intrahepatic cholangiocarcinoma (ICC), oligodendrogliomas, astrocytomas, glioblastomas, leukemias, adenocarcinomas, gliomas, melanomas, oligoastrocytomas, invasive breast carcinomas, invasive ductal carcinomas, and myelodysplastic syndromes.

### BACKGROUND OF THE DISCLOSURE

Isocitrate dehydrogenase 1 and/or 2 (IDH1/2) mutation(s) are associated with a number of adverse conditions. For example, IDH1/2 mutation(s) occur in intrahepatic cholangiocarcinoma (ICC), an aggressive cancer with a 5-year survival rate of only 24% if caught in the early stages. However, early stages of ICC are often asymptomatic, and the majority of cases present without identifiable risk factors. Despite significant advances in understanding cholangiocarcinoma etiology, diagnosis, and treatment, the 5-year survival rate for regional ICC has remained dismal at 9%, with an overall median survival of 11.7 months and median progression-free survival of 8.0 months.

Gemcitabine/cisplatin combination chemotherapy is the first line therapy for unresectable ICC, with folinic acid, fluorouracil and oxaliplatin (FOLFOX) as a second line therapy. More recently, the U.S. Food and Drug Administration has approved durvalumab in combination with gemcitabine and cisplatin due to an increased survival rate. However, there are no effective treatment options available for patients with ICC or other conditions associated with IDH1/2 mutation(s) that do not respond to the current standard of care.

### SUMMARY OF THE DISCLOSURE

The current disclosure provides compositions and methods for the treatment of conditions associated with IDH1/2 mutation(s). The compositions and methods include administration of an SRC inhibitor and a p70 S6 kinase/AKT (S6K/AKT) inhibitor. In particular embodiments, SRC inhibitors include: dasatinib, saracatinib, bosutinib, ponatinib, tirbanibulin, NXP900, KX01, KX2-391, PP1, or PP2. In particular embodiments, S6K/AKT inhibitors include: M2698, pyrazolopyrimidines, LY2780301, LY2584702, GNE-477, paxalisib, pyrvinium pamoate, PF-4708671, or MSC2363318A.

Examples of such conditions associated with IDH1/2 mutation(s) include intrahepatic cholangiocarcinoma (ICC), oligodendrogliomas, astrocytomas, glioblastomas, leukemias, adenocarcinomas, gliomas, melanomas, oligoastrocytomas, invasive breast carcinomas, invasive ductal carcinomas, and myelodysplastic syndromes.

In some aspects, treatment of conditions associated with IDH1/2 mutation(s) may be based on levels of S6 phosphorylation in subjects. For example, in some aspects, combinations of an SRC inhibitor and an S6K/AKT inhibitor may be administered to a subject with an elevated level of phosphorylated S6 in comparison to a reference level of phosphorylated S6.

To the accomplishment of the foregoing and related ends, certain illustrative aspects of the compositions and methods are described herein in connection with the following description and the attached drawings. The summary is provided to introduce a selection of concepts in a simplified form that are further described below in the Detailed Description.

### BRIEF DESCRIPTION OF THE SEVERAL VIEWS OF THE DRAWINGS

Some of the drawings submitted herewith may be better understood in color. Applicant considers the color versions of the drawings as part of the original submission and reserves the right to present color images of the drawings in later proceedings.

FIGS. 1A, 1B are (FIG. 1A) proliferation and (FIG. 1B) survival curves of human Isocitrate Dehydrogenase (IDH) wild type (WT) (HuCCT1, CCLP1, and ICC2) v. mutant (m) (SNU-1079, RBE, and ICC5) intrahepatic cholangiocarcinoma (ICC) cell lines treated with increasing doses of dasatinib.

FIG. 2 is a graph depicting the results of an apoptosis assay measuring the percentage of IDH WT (HUCCT) and IDHm (RBE, ICC5, and SNU) cells stained positive for annexin V by flow cytometry at 48 hours (h) after dasatinib treatments. Data are shown as mean±standard error of the mean (SEM) between triplicates and are representative of three independent experiments (Student's two-tailed t test). **P<0.01; ***P<0.001; ****P<0.0001. ns, not significant. N.D., not defined.

FIG. 3A-3C include (FIG. 3A) immunofluorescence staining showing that dasatinib inhibits phosphorylated protein S6 (pS6) in IDHm, but not IDH WT, ICC cell lines. RBE, SNU1079, ICC5 (IDH1 mutant (IDH1m)) and HuCCT1 (IDH1 WT) cells were treated with 50 nM dasatinib for 6 h and subjected to immunofluorescent staining for pS6 and total S6. Quantifications of pS6 signal in IDH WT cells are shown in FIG. 3B and in IDHm ICC cells are shown in FIG. 3C. Ns, not significant; ****, p<0.0001.

FIGS. 4A, 4B are western blots of (FIG. 4A) WT or mutant ICC cell lines that were treated with 50-500 nM of dasatinib for 6 h and probed for the indicated marks at mTOR, ERK, and STAT3 survival pathways; and (FIG. 4B) RBE and HuCCT1 cells that were treated with increasing doses of dasatinib (50, 100, 250, 500 nM) for 6 h and analyzed for the change in phosphorylation of RSK and S6K2.

FIGS. 5A, 5B are forward scatter plots showing the percent reduction in cell size of WT or mutant ICC cells treated with DMSO (indicated by a triangle) or 100 nM dasatinib (indicated by a diamond) for 24 h as measured by flow cytometry in (FIG. 5A) HuCCT1 and (FIG. 5B) RBE cell lines.

FIGS. 6A, 6B illustrate (FIG. 6A) a graph comparing cell size change in RBE, SNU-1079, ICC5, HuCCT1, CCLP, and ICC2 cell lines treated with 100 nM dasatinib (*, p<0.05; **, p<0.01; ***, p<0.001; ****, p<0.0001); and (FIG. 6B) annexin V apoptosis assay results of RBE parental and RBE SRC T341I cells that were treated with 50 nM dasatinib or DMSO for 24 h and 48 h (Two-way ANOVA, ns, not significant; ****, p<0.0001).

FIG. 7 is a western blot of indicated marks or puromycin-labeled proteins in representative WT and mutant ICC cells treated as in FIG. 4A. Thirty minutes before harvest, cells were exposed to 1 μM puromycin. Lysates were probed for the indicated proteins or puromycin-labeled proteins by Western blot.

FIG. 8 is a western blot showing that treatment of dual mTORC1 and mTORC2 inhibitor Torin1 resulted in pS6K and pS6 reduction in IDH WT and IDHm ICC cells at a comparable level.

FIG. 9 is a western blot showing that treatment of dual mTORC1 and mTORC2 inhibitor AZD2014 resulted in pS6K and pS6 reduction in IDH WT and IDHm ICC cells at a comparable level.

FIGS. 10A, 10B, 11A, 11B, and 12-14 illustrate that inhibition of proto-oncogene tyrosine-protein kinase Src (SRC) is both necessary and sufficient in killing IDHm ICC through inhibition of the S6K/S6 axis.

FIGS. 10A, 10B are proliferation curves of parental IDH1 m ICC cell lines (indicated by open squares) or isogenic lines harboring a genomic SRCT341I gatekeeper mutation rendering endogenous SRC dasatinib-resistant (indicated by triangles) treated with increasing doses of dasatinib in (FIG. 10A) RBE and (FIG. 10B) SNU-1079 cell lines.

FIGS. 11A, 11B are graphs of parental SRC WT and SRC gatekeeper lines treated with dasatinib at the indicated doses for 48 h and assessed for the induction of apoptosis by flow cytometry as seem in (FIG. 11A) RBE and (FIG. 11B) SNU-1079 cell lines.

FIG. 12 is a western blot showing the indicated proteins in mTOR, ERK, and STAT3 survival pathways in cells treated with 50-500 nM of dasatinib for 6 h.

FIG. 13 is a western blot of cells treated as in FIG. 12 and, thirty minutes before harvest, exposed to 1 μM puromycin. Lysates were probed for the indicated proteins or puromycin-labeled proteins.

FIG. 14 is a western blot of the indicated antibodies in IDH WT (ICC2, HuCCT1, and CCLP1) or IDHm (ICC5, RBE, and SNU-1079) ICC cell lines that were transduced with either a control shRNA or two independent shRNAs against SRC. Lysates were probed with antibodies of the indicated proteins by western blot.

FIG. 15 illustrates Search Tool for the Retrieval of Interacting Genes/Proteins (STRING) analysis of SRC interaction with other involved mTOR downstream molecules. SRC, S6K1, S6 (RPS6), mTOR, 4EBP1, PPP2CA, PPP2CB, PPP2R5C and MAGI1 were subjected to STRING analysis for known protein-protein interaction. Network nodes represent proteins, whereas edges represent protein-protein associations (known or predicted). Symbols adjacent to the edges represent the types of interaction evidence as shown in the bottom key. Only protein-protein association with higher than STRING calculated combined score of 0.7 (high confidence) is shown.

FIGS. 16A, 16B depict the identification of SRC substrates by phosphoproteomic screen in which IDH1 m ICC SRC WT and T341I gatekeeper pairs were treated with 20 nM dasatinib for 1 h. Phosphopeptides were extracted from the tryptic digests of the protein lysates, followed by mass spectrometry-based multiplexed quantitative phosphoproteomics with phospho-tyrosine peptide, pMAGI Y373, representing the top candidate that was inhibited by dasatinib in both SRC WT lines, (FIG. 16A) SNU-1079 and (FIG. 16B) RBE, but not in their corresponding SRC gatekeeper lines.

FIGS. 17A, 17B are graphs depicting the relative intensity of (FIG. 17A) pMAGI1 Y373 and (FIG. 17B) total MAGI1 signals in IDH1m (SNU-1079 and SNU-1079 SRC T341 I) and WT IDH1 (HuCCT1 and CCLP1) ICC lines treated with DMSO or dasatinib by phosphoproteomic screen.

FIG. 18 is a cartoon of the domain structure of MAGI1 showing the pMAGI1 Y373 site.

FIG. 19 is a western blot of 293T cells transfected with either vector control, SRC, GFP-tagged MAGI1 WT, or GFP-MAGI1 Y373F. Lysates were probed with rabbit antisera against pMAGI1 Y373 or the indicated antibodies.

FIG. 20 is a western blot of pMAGI1 Y373 antisera in 293T cells transfected with either vector control, SRC, myc-tagged MAGI1 full length, or myc-tagged MAGI1 deletion of GUK, WW, or GUK-WW domains.

FIG. 21 is a western blot of 293T cells transfected with either vector control, SRC, flag-tagged MAGI1 full length, MAGI1 GUK deleted, MAGI1 WW deleted, or MAGI1 GUK-WW deleted truncation mutants or co-transfected with SRC, flag-tagged MAGI1 full length, and truncation mutants. Lysates were then immunoprecipitated (IP) with flag antibody and analyzed by Western Blot along with 1% input and probed for SRC and flag antibodies.

FIG. 22 is a western blot of antibodies in IDH1m ICC cells treated with control siRNA or siRNA against MAGI1 and then exposed to increasing doses of dasatinib (5-50 nM) for 6 h.

FIG. 23 is a western blot of antibodies in IDH1m cells RBE expressing Cas9 plus either control sgRNA or sgRNA targeting MAGI1 (clone 2 and clone 4) and then treated as in FIG. 22.

FIG. 24 is a western blot of the same control and MAGI1 knockout clones 2 and 4 as in FIG. 23 that were treated with dasatinib at indicated doses for 48 h and subjected to annexin V apoptosis assay (two-way ANOVA; **P<0.01; ***P<0.001; ****P<0.0001).

FIG. 25 is a western blot showing MAGI1 silencing and mTOR signaling in ICC cells. Three pairs of IDH1 WT (ICC2, HuCCT1, and CCLP1) and IDH1m (ICC5, RBE, and SNU-1079) human ICC cell lines were treated with non-targeting siRNA control or MAGI1 siRNA for two days and the changes of activities in mTOR, STAT3, ERK survival signaling were examined by western blot.

FIGS. 26A, 26B illustrate the effect of S6K overexpression on SRC signaling in IDHm cells treated with dasatinib. RBE cells stably overexpressing (FIG. 26A) p70 S6K or empty vector, or (FIG. 26B) p70 and p85 S6K or empty vector, were treated with increasing doses of dasatinib for 6 h, and the change of SRC, S6K and S6 signaling was examined by western blot.

FIGS. 27, 28, 29A-29C, 31A, and 31B illustrate that dasatinib suppresses S6K signaling through induction of protein phosphatase 2A.

FIG. 27 is a western blot of RBE, SNU-1079 and ICC5 cells treated with dasatinib 100 nM for 6 h and/or the PP2A inhibitor okadaic acid for the indicated timepoints prior to harvest. Cells were probed for phospho- and total SRC, S6K, S6, 4E-BP1 with quantification of pS6K and pS6 are presented below each blot and normalized with corresponding total protein.

FIG. 28 is a western blot of IDH1 m ICC cells treated with control siRNA or siRNA against PPP2CA and then exposed to increasing doses of dasatinib (5-50 nM) for 6 h.

FIGS. 29A-29C are graphs of mRNA expressions of PPP2CA and PPP2CB by qRT-PCR in (FIG. 29A) RBE, (FIG. 29B) SNU-1079, and (FIG. 29C) ICC5 cell lines confirming specific depletion of PPP2CA by siRNA.

FIG. 30 is a western blot showing SRC, S6K, and S6 signaling in RBE parental and RBE Cas9 MAGI1 knockout cells stably re-expressing either MAGI1 WT or MAGI1 Y373E 6hosphor-mimetic mutant. Cells were treated with DMSO or indicated doses of dasatinib for 6 h.

FIGS. 31A, 31B are western blots of a change in PP2A catalytic subunit post translational modification at pY307 in (FIG. 31A) two IDH1 WT and mutant pairs and (FIG. 31B) two SRC WT and SRC T341I gatekeeper pairs in IDH1 m lines upon increasing doses of dasatinib (50-500 nM).

FIGS. 32A-32E illustrate that SRC interacts with the MAGI1-PP2A complex to regulate S6K. FIGS. 32A-32E include western blots of (FIG. 32A) 293T cells that were co-transfected with WT SRC and flag-tagged MAGI1 Y373 WT/Y307E/Y307F for 2 days and treated with DMSO or 50 nM dasatinib for 6 h prior to lysis. Lysates were then immunoprecipitated with flag antibody and analyzed by Western blot along with 1% input and probed for SRC and flag antibodies. (FIG. 32B) Western blot of 293T cells that were transfected with either vector control, HA-tag PP2A catalytic subunit (PP2AC), flag-tagged MAGI1 Y373 WT, flag-tagged MAGI1 Y373E, flag-tagged MAGI1 Y373F or co-transfected with HA-PP2AC and flag-tagged MAGI1 Y373 WT/E/F mutants and treated with DMSO or dasatinib before harvesting for co-immunoprecipitation (co-IP) as in FIG. 32A. Cells were analyzed by Western blot with HA and flag antibodies. Western blots of (FIG. 32C) 293T cells transfected with HA-tag PP2AC and flag-tagged MAGI1 full length or without WW, GUK or GUK-WW domains and immunoprecipitated with flag antibody; and (FIG. 32D) flag-tagged MAGI1 full length WT and HA-PP2AC Y307 WT/E/F mutants co-transfected in 293T cells treated with DMSO or dasatinib for 6 h and processed for IP as in FIG. 32A. HA-tagged PP2AC and endogenous S6K pulled down were analyzed by western blot. (FIG. 32E) 293T cells co-transfected with HA-tag S6K and flag-tagged MAGI1 Y373 WT/Y307E/Y307F and treated with DMSO or dasatinib before harvesting for co-IP as in FIG. 32A.

FIG. 33 is a western blot showing co-IP of PP2AC-HA and MAGI1 WT-flag in 293T cells treated with DMSO or increasing doses of dasatinib (50, 100, 250, 500 nM) for 6 h.

FIGS. 34A, 34B are cartoons of the MAGI1-PP2A tumor suppressive complex regulating IDHm cells survival.

FIGS. 35A-35D and 36-43 illustrate that 2-HG is required in dasatinib-induced cytotoxicity and inhibition of pS6K.

FIGS. 35A-35D illustrate (FIG. 35A) immunofluorescence staining of pS6 and total S6 in RBE, SNU1079, ICC 5 (IDH1m), and HuCCT1 (IDH1 WT) cells (scale bars, 50 μm); and (FIG. 35B) quantification of pS6 median staining intensity. Each data point represents one cell (one-way ANOVA test, ****P<0.0001). (FIG. 35C) Immunohistochemistry staining of pS6 in samples from patients with IDH WT or IDH1 R132C ICC (scale bars, 50 μm); and (FIG. 35D) quantification of pS6 IHC staining intensity by blinded histopathology scoring (one-way ANOVA test, *P<0.05).

FIG. 36 depicts IC50 curves of IDH1 m-specific inhibitor AG-120 in suppressing cellular 2-HG concentrations in three IDHm (SNU-1079, RBE, and ICC5) and IDH WT (RBE KI c5 and RBE KI c9) cell lines.

FIG. 37 depicts proliferation curves of IDH1m (SNU-1079 and RBE) and WT (HuCCT1 and SSP25) lines with increasing doses of AG-120.

FIG. 38 is an image of SNU-1079 cells that were treated with regular media, DMSO, or 5 μM AG-120 for 1, 2, 3 or 4 days followed by dasatinib treatment for 72 h at indicated doses and subjected to crystal violet staining.

FIG. 39 illustrates results of an annexin V apoptosis assay of the cells shown in FIG. 38 (Student's two-tailed t test,**, p<0.01; ****, p<0.0001).

FIG. 40 is a western blot of apoptosis markers, cleaved caspase-3 and PARP, of the cells illustrated in FIG. 38.

FIG. 41 is a western blot of markers in IDH1 m ICC cells pre-treated with either DMSO or 5 μM AG-120 for four days, followed by DMSO or increasing doses of dasatinib (5 nM-50 nM) for 6 h.

FIG. 42 is a western blot of IDH1 m RBE cells and their isogenic WT knocked in (KI) clone 9 that were subjected to increasing doses of dasatinib (50 nM-500 nM) treatment for 6 hours.

FIG. 43 is a western blot of the baseline expression of pSRC, pS6K, and pS6 in a human ICC cell line panel.

FIGS. 44A-44D are IC50 curves of dasatinib-resistant clones to SRC kinase inhibitors, including (FIG. 44A) dasatinib, (FIG. 44B) saracatinib, (FIG. 44C) bosutinib, and (FIG. 44D) ponatinib. Parental RBE cells and two RBE dasatinib resistant clones were treated with increasing doses of dasatinib, saracatinib, bosutinib, and ponatinib for 7 days, and cell viabilities were measured by MTT assay.

FIG. 45 is a western blot of dasatinib resistant clones generated through continuous dasatinib treatment that were subjected to increasing doses of dasatinib treatment.

FIGS. 46A-46C are IC50 curves of IDH WT and mutant cells lines to S6K inhibitors. IDHm cells (SNU-1079 and RBE) and IDH WT cells (CCLP1 and HuCCT1) were treated with S6K inhibitors M2698 (FIG. 46A), PF-4708671 (FIG. 46B), and LY2584702 (FIG. 46C) for 7 days, and the cell viabilities were measured by MTT assay. IC50 values of the three drugs in these cells lines were summarized in Table 4.

FIGS. 47, 48, 49A, 49B, and 50A-50F illustrate that combination treatment with SRC and S6K inhibitors suppresses IDHm ICC growth in patient-derived models.

FIG. 47 is a western blot of IDH WT and mutant ICC cells that were treated with increasing doses of dasatinib (50 nM-500 nM) with or without S6K1/AKT inhibitor M2698 at 10 nM for 6 h, lysed, and probed for the indicated markers.

FIG. 48 is a western blot of pS6k and pS6 levels in patient-derived organoid ICC195, FHICC19 (WT), and FHICC17 (IDH1 m) cells that were treated with increasing doses of dasatinib (100, 500, 1000 nM) for 16 h in the first three panels and, in the fourth panel, FHICC17 IDH1m organoids treated with combination dasatinib and S6K1/AKT inhibitor M2698 at 20 nM for 16 h.

FIGS. 49A, 49B are quantifications of (FIG. 49A) pS6 levels in dasatinib-treated organoid lines ICC 195, FHICC 19, and FHICC 17, and (FIG. 49B) pS6 levels in dasatinib plus M2698-treated FHICC 17 IDH1m organoids.

FIGS. 50A-50F illustrate experimental results from non-obese diabetic (NOD) severe combined immunodeficiency mice (SCID) gamma (NSG) mice with subcutaneously implanted IDH1 WT PDX (ICC 195) and IDH1 R132C mutant PDXs (PDX62) that were treated with vehicle control, dasatinib (30 mg/kg), M2698 (10 mg/kg), or dasatinib (30 mg/kg)+M2698 (10 mg/kg) daily for 28 days by oral gavage (PDX62, n=9, 10, 9, and 12 respectively; ICC 195, n=5 each arm). Part of the IDH1 m PDX cohort was harvested at day 28 of treatment (n=3, 4, 3, and 4 for vehicle, dasatinib, M2698, and dasatinib+M2698 respectively), and the remaining mice were monitored for survival up to 70 days after treatment. Tumor volume fold change of IDH1 m PDX (FIG. 50A) and IDH1 WT (FIG. 50B), tumor volume change at day 29 compared with day 1 for IDH1 m PDX (FIG. 50C), and survival plot for IDH1 m PDX (Kaplan-Meier analysis and log-rank P values are shown between groups) (FIG. 50D). (FIG. 50E) Histological analysis of tumors from PDX62 vehicle, dasatinib, M2698, and combination groups. Left column: Hematoxylin and eosin (H&E) staining; middle column: IHC staining for Ki67 (proliferation marker); and right column: IHC staining for cleaved caspase-3 (cell death marker). Representative images of each experimental group are shown. Scale bar, 250 μm. (FIG. 50F) Quantification of Ki67 (top) and cleaved caspase-3 (bottom) IHC staining. Each dot represents the percentage of cells with positive staining in a randomly selected area. Five areas per slide were quantified. Data are mean±SEM (one-way ANOVA multiple comparisons; *P<0.05; ****P<0.0001).

FIGS. 51A, 51B show mouse weight in (FIG. 51A) NSG mice with subcutaneously implanted IDH1 WT PDX (PDX ICC 195) and (FIG. 51B) NOD SCID mice with IDH1m PDXs (PDX62) treated with vehicle control, dasatinib 30 mg/kg, M2698 10 mg/kg, or dasatinib 30 mg/kg+M2698 10 mg/kg daily by oral gavage.

FIG. 52 includes exemplary sequences supporting the disclosure: Guide RNA sequence, TCATAGGTCGTCATGCTTAT (SEQ ID NO. 1); Human shSRC #1 (TRCN0000195339) CATCCTCAGGAACCAACAATT (SEQ ID NO: 2); shSRC #2 (TRCN0000199186) target sequence CTGACTGAGCTCACCACAAAG (SEQ ID NO: 3); pLKO.1 shRNA with target sequence GCAAGCTGACCCTGAAGTTCAT (SEQ ID NO:4); pCMV6-AC-GFP-MAGI1c WT (Origene, #RG212712) CATAGTAGATACCAAAGACAGGGTCTTCAATCTTTTCCCAAC (SEQ ID NO: 5); pCMV6-AC-GFP-MAGI1c WT (Origene, #RG212712) GTTGGGAAAAGATTGAAGACCCTGTCTTTGGTATCTACTATG (SEQ ID NO: 6); Guide RNA sequence used to generate human MAGI1 KO in Cas9 expressing stable RBE cells G*A*A*GGGUUUCGUGUAAAAAA (SEQ ID NO: 7); Guide RNA sequence used to generate human MAGI1 KO in Cas9 expressing stable RBE cells A*U*C*AAGAGCUUGGUCCUAGA (SEQ ID NO: 8); Guide RNA sequence used to generate human MAGI1 KO in Cas9 expressing stable RBE cells*C*G*UGGCUUUGGCUUCACGG (MAGI1) (SEQ ID NO: 9); Guide RNA sequence used to generate human MAGI1 KO in Cas9 expressing stable RBE cells G*C*A*CUACCAGAGCUAACUCA (non-targeting control) (SEQ ID NO: 10).

### DETAILED DESCRIPTION

The current disclosure provides compositions and methods for treatment of conditions associated with Isocitrate Dehydrogenase (IDH) 1 and/or 2 (1/2) mutation(s).

IDH is a metabolic enzyme present in the cytoplasm and mitochondria that reduces isocitrate to α-ketoglutarate. Hotspot mutations in the isocitrate binding domain of IDH are known to promote cancers in many cell lineages through gain-of-function enzymatic activity, which produces R (−)-2-hydroxyglutarate (2-HG), an oncometabolite. 2-HG inhibits α-ketoglutarate dependent dioxygenases, which are often epigenetic modifiers, and contributes to large-scale changes in the genomic landscape, ultimately resulting in impaired cholangiocyte differentiation.

There are three isoforms of IDH: IDH1, IDH2, and IDH3. IDH1 localizes to the cytoplasm and peroxisomes, and IDH2 and IDH3 localize to the mitochondrial matrix. IDH mutations associated with cancer are generally at the arginine residue responsible for recognition of the substrate, isocitrate. For instance, the arginine residue at position 132 of IDH1 is often replaced with a lower-polarity amino acid, such as histidine, lysine, or cysteine (Han, et al., Br J Cancer 122, 1580-9 (2020). There are two common mutations of IDH2: the arginine residues at position 140 and at position 172. These are often replaced with glutamine, serine, threonine, lysine, or methionine (Guo, et al., Front Oncol 11, 644857 (2021)). These missense mutations of IDH1 and IDH2 cause reduced transformation of isocitrate into α-KG and the production of 2-HG.

IDH1/2 mutations are present in 18%-37% of intrahepatic cholangiocarcinoma (ICC) cases in North America and Europe. Additional examples of conditions associated with IDH1/2 mutation(s) include oligodendrogliomas (e.g., anaplastic oligodendroglioma), astrocytomas (e.g., anaplastic astrocytoma and diffuse astrocytoma), glioblastomas (e.g., conventional glioblastoma multiforme), leukemias (e.g., acute myeloid leukemia), adenocarcinomas (e.g., lung adenocarcinoma, colon adenocarcinoma, endometrial endometrioid adenocarcinoma, high grade ovarian serous adenocarcinoma, and pancreatic adenocarcinoma), gliomas (e.g., diffuse glioma and high-grade glioma), melanomas (e.g., cutaneous melanoma), oligoastrocytomas (e.g., anaplastic oligoastrocytoma), invasive breast carcinomas, invasive ductal carcinomas, oligodendrogliomas, and myelodysplastic syndromes.

The disclosed compositions and methods include administering an SRC inhibitor and a p70 S6 kinase/AKT (S6K/AKT) inhibitor to a subject who has a condition associated with one or more IDH1/2 mutation(s).

SRC is a non-receptor tyrosine kinase that drives aggressiveness and poor prognosis in a number of cancers. SRC activity is generally modulated by phosphorylation events at Y416 and Y527, which are activating and inhibitory, respectively (chicken numbering is used for consistency, however, these would be Y419 and Y530, respectively, in humans). SRC is a well-described proto-oncogene and is involved in a number of signaling cascades controlling differentiation, angiogenesis, motility, and proliferation.

SRC is reported to inhibit protein phosphatase 2A (PP2A) through phosphorylating its catalytic subunit (Yokoyama and Miller, FEBS Lett 505, 460-464 (2001)). The PP2A holoenzyme is composed of three subunits, A (structural), B (regulatory) and C (catalytic). The human genome encodes only two distinct PP2A-A subunits and two distinct PP2A-C subunits but there are at least 12 different PP2A-B genes that allow for a variety of regulatory mechanisms and substrate specificities (Sangodkar, et al., FEBS J 283, 1004-1024 (2016)).

An SRC inhibitor is a compound that targets a member of the SRC family of tyrosine kinases and inhibits one or more cellular processes of a SRC family of tyrosine kinase(s), such as cell growth, differentiation, cell shape, migration and survival, and specialized cell signals. Examples of SRC inhibitors include: dasatinib, saracatinib, bosutinib, NXP900, KX01, KX2-391, PP1, and PP2.

S6K is a member of the cAMP-dependent protein kinase, cGMP-dependent protein kinase, and protein kinase C families (AGC) kinase family that has been implicated in obesity, diabetes, aging, and various types of cancer. S6K activity is predominantly regulated by phosphorylation events at multiple sites, including S411, S418, T421, and S424. In particular, phosphorylation at S411 and S424 allows S6K activation via the oncogenic PI3K/AKT/mTOR (PAM) pathway. Dysregulation of S6Ks has been associated with protein synthesis, mRNA processing, insulin resistance, and cell size. There are two S6K genes in humans, S6K1 and S6K2, which each give rise to two distinct protein isoforms. The most studied isoform is p70 S6K, which is an isoform of S6K1 with a length of 502 amino acids that is found in both the nucleus and the cytoplasm (Fenton and Gout, Int J Biochem Cell Biol 41, 47-59 (2011)). The p70 isoform is a downstream effector of the PAM pathway, which is associated with many cellular functions, including cell cycle regulation, cell growth, and cell metabolism (Artemenko, et al., Cancer Lett 535, 215593 (2022)).

AKT, also known as protein kinase B (PKB), is also a member of the AGC kinase family that is often constitutively active in a number of cancers. Constitutive AKT signaling has been associated with cell proliferation and increased cell survival (Nicholson and Anderson, Cell Signal 14, 381-95 (2002)). AKT has been implicated in cell growth, cell metabolism, apoptosis suppression, and angiogenesis. Dysregulation of AKT-related pathways are associated with cancer, diabetes, cardiovascular diseases, and neurological diseases. AKT can be activated by PIP3, which alters the conformation of AKT to facilitate phosphorylation at T308, and by a second phosphorylation event at S473 (Nitulescu, et al., Int J Oncol 53, 2319-31 (2018)). AKT has been found to modulate downstream protein activity by phosphorylating Ser/Thr residues within a minimal consensus recognition motif of R-α-R-α-α-S/T-β (where a is any amino acid and p denotes a preference for large hydrophobic residues) (Manning and Toker, Cell 169, 381-405 (2017)).

An S6K inhibitor is an inhibitor that targets one or more members of the AGC kinase family and inhibits one or more cellular processes of the AGC kinase family such as cell growth regulation, and phosphorylation of proteins involved in RNA processing. For example, an S6K inhibitor may inhibit one or more cellular processes of the S6K1 protein, such as cell growth regulation. An AKT inhibitor inhibits one or more cellular processes of serine/threonine kinase AKT, a component of the PI3K/AKT/mTOR signaling pathway that is involved in cell growth, proliferation, survival, and metabolism. An S6K/AKT inhibitor is a dual inhibitor that targets both S6K and AKT and inhibits one or more cellular processes of serine/threonine kinase AKT and S6K1. Examples of S6K/AKT inhibitors include: M2698, pyrazolopyrimidines, LY2780301, LY2584702, GNE-477, paxalisib, pyrvinium pamoate, PF-4708671, and MSC2363318A

A high-throughput drug screen of a large panel of cancer cell lines, including 17 biliary tract cancers, found that IDH mutant (IDHm) ICC cells demonstrate a striking response to the multikinase inhibitor dasatinib. As described in further detail below, dasatinib, a multi-kinase, SRC, and BCR-ABL inhibitor, induced apoptosis in IDHm ICC cell lines but not in IDH wild-type cell lines. Further, dasatinib induced necrosis in an IDHm patient-derived xenograft (PDX) model and was also effective in reducing tumor burden in a genetically modified mouse model harboring an IDH2 mutation. Using a multiplexed inhibitor bead column followed by active kinome profiling, the direct target of dasatinib in IDHm ICC was confirmed to be SRC (Hertz, et al., Curr Protoc Chem Biol 2, 15-36 (2010)).

As shown in the experimental results herein, dasatinib inhibits SRC equally in IDH WT and mutant cells, but S6K and S6 phosphorylation are only reduced in the mutant counterparts. Without being bound by theory, upon SRC inhibition, a scaffolding molecule, membrane associated guanylate kinase, WW and PDZ Domain Containing 1 WW and PDZ Domain Containing 1 (MAGI1) forms a tumor suppressive complex with PP2A facilitating S6K dephosphorylation and suppression of S6K/S6 signaling. In IDHm cells, SRC was found to be active and phosphorylate MAGI1 at Y373 and PP2AC at Y307, inhibiting both PP2A phosphatase activity and the formation of the MAGI1-PP2A tumor suppressive complex leading to downstream activation of S6K/S6 survival signaling.

Treatment of malignancies with rapamycin, a specific inhibitor of mTORC1, has shown differential regulation of 4E-BP1 and S6K, in a cell-specific manner. mTOR controls protein synthesis, at least in part, through direct phosphorylation of the tumor suppressor eukaryotic translation initiation factor 4E-BP1 and S6K. Without being bound by theory, inhibition of SRC by dasatinib treatment inhibits p70S6 kinase (S6K) and ribosomal protein S6 (S6), members of the mTORC1 pathway, exclusively in IDHm cells and reduces cell size and de novo protein synthesis. Upon SRC inhibition, MAGI1 forms a tumor suppressive complex with Protein Phosphatase 2A (PP2A) facilitating S6K dephosphorylation and suppression of S6K/S6 signaling.

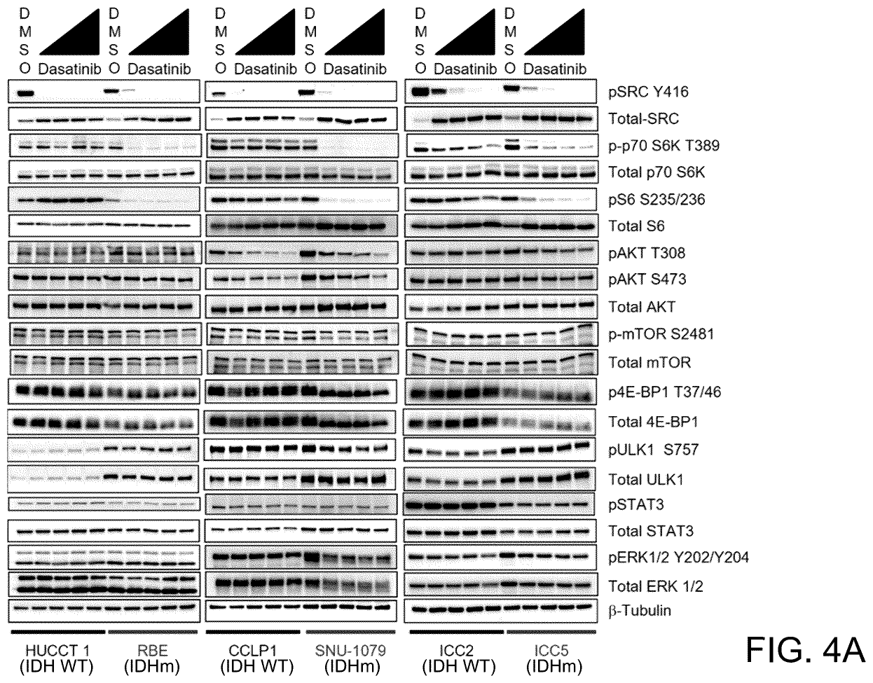

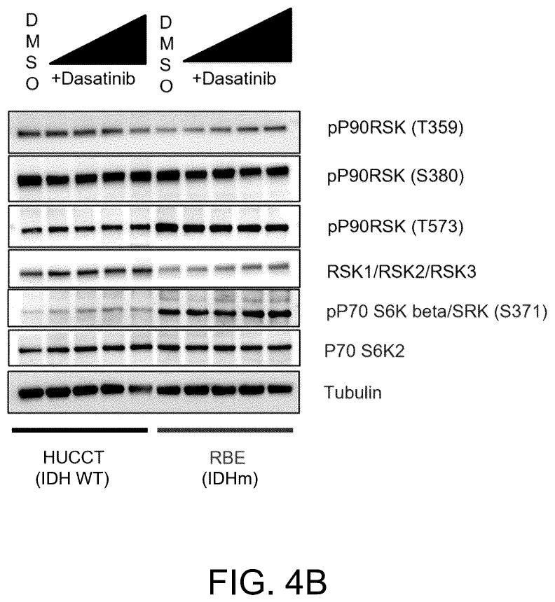

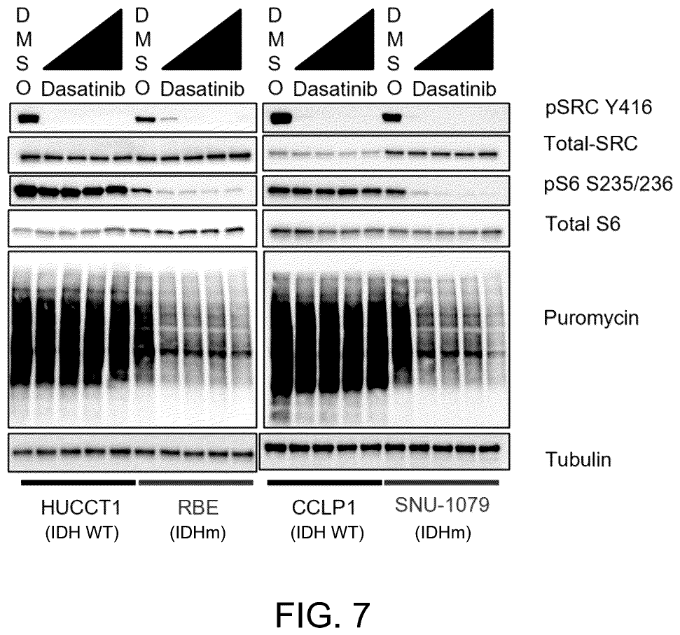

Based on the foregoing, in some aspects, administration of dasatinib to a patient in need thereof may reduce cell size in comparison to patients with IDH1/2 malignancies who have not been administered dasatinib (FIG. 4A). In particular embodiments, administration of dasatinib to a patient in need thereof may reduce protein synthesis in comparison to patients with IDH1/2 malignancies who have not been administered dasatinib (FIG. 7). In particular embodiments, administration of dasatinib to patients in need thereof may inhibit PP2A phosphatase activity. As shown in the examples below, SRC phosphorylation of MAGI1 Y373 and PP2A Y307 may prevent the formation of a suppressive MAGI1-mediated signaling complex. This limits access of PP2AC to S6K and leads to S6K/S6 hyperactivation.

In some aspects, patients with IDH1/2 mutation(s) associated conditions may develop dasatinib resistance.

In some aspects, patients with IDH1/2 mutation(s) associated conditions may be tested for levels of phosphorylated protein S6 or phospho-pS6 (pS6). Elevated levels of pS6 in comparison to threshold levels may indicate resistance of a given cancer to SRC inhibitors alone. Such patients may be treated with a combination of an SRC inhibitor and an S6K/AKT inhibitor.

In some aspects the combination of the SRC inhibitor and S6K/AKT inhibitor may allow for effectiveness at lower doses than the administration of each inhibitor alone.

Aspects of the current disclosure are now described in with additional details and options as follows: (i) Compositions and Formulations; (ii) Methods of Use; (iii) Reference levels; (iv) Exemplary Embodiments; (v) Experimental Examples; and (vi) Closing Paragraphs. These headings are provided for organizational purposes only and do not limit the scope or interpretation of the disclosure.

(i) Compositions and Formulations. In particular embodiments, an SRC inhibitor and an S6K/AKT inhibitor (collectively active compounds) can be formulated together or separately into compositions for administration. When active compounds are formulated separately into compositions that are administered separately, such compounds may be administered in sequence, in either order, or at the same time (concurrently, whether formulated into a single composition or administered as separate compositions).

Active compounds can be formulated into compositions for delivery with a pharmaceutically acceptable carrier that is suitable for administration to a subject. Pharmaceutically acceptable carriers include those that do not produce significantly adverse, allergic or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, compositions can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

For injection, compositions can be made as aqueous solutions, such as in buffers such as Hanks' solution, Ringer's solution, or physiological saline. The solutions can contain formulatory agents such as suspending, stabilizing and/or dispersing agents. Alternatively, the composition can be in lyophilized and/or powder form for constitution with a suitable vehicle, e.g., sterile pyrogen-free water, before use.

Therapeutically effective amounts of active compounds within a composition can include at least 0.1% w/v or w/w active compounds; at least 1% w/v or w/w active compounds; at least 10% w/v or w/w active compounds; at least 20% w/v or w/w active compounds; at least 30% w/v or w/w active compounds; at least 40% w/v or w/w active compounds; at least 50% w/v or w/w active compounds; at least 60% w/v or w/w active compounds; at least 70% w/v or w/w active compounds; at least 80% w/v or w/w active compounds; at least 90% w/v or w/w active compounds; at least 95% w/v or w/w active compounds; or at least 99% w/v or w/w active compounds.

Exemplary preservatives include phenol, benzyl alcohol, meta-cresol, methyl paraben, propyl paraben, octadecyl dimethyl benzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the antibodies or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinositol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, and/or intravesicular administration.

Additionally, compositions can be formulated as sustained-release systems utilizing semipermeable matrices of solid polymers including at least one type of antibody. Various sustained-release materials have been established and are well known by those of ordinary skill in the art. Sustained-release systems may, depending on their chemical nature, release one or more antibodies following administration for a few weeks up to over 100 days. Depot preparations can be administered by injection; parenteral injection; instillation; or implantation into soft tissues, a body cavity, or occasionally into a blood vessel with injection through fine needles.

Any composition or formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers are disclosed in Remington's Pharmaceutical Sciences, 18th Ed. Mack Printing Company, 1990. Moreover, compositions and formulations can be prepared to meet sterility, pyrogenicity, general safety, and purity standards as required by U.S. FDA Office of Biological Standards and/or other relevant foreign regulatory agencies.

(ii) Methods of Use. Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with compositions disclosed herein.

In particular embodiments, the subject has symptoms of a condition associated with IDH1/2 mutation(s). In particular embodiments, the condition associated with IDH1/2 mutation(s) is a cancer. In particular embodiments, a subject is at risk for metastasis of a cancer associated with IDH1/2 mutation(s). In particular embodiments, the subject previously had a cancer associated with IDH1/2 mutation(s) and is at risk for recurrence.

In particular embodiments, the methods include identifying a subject in need of a therapeutic or preventative treatment for a hyperproliferative disease. In some aspects, methods may include identifying IDH1 and/or IDH2 (IDH1/2) mutation(s) in a subject. IDH1/2 mutation(s) may be determined using any method generally known to those of ordinary skill in the art. For example, IDH1/2 mutation(s) may be determined using direct nucleic acid sequencing, hybridization methods, restriction enzyme digestion, polymerase chain reaction (PCT) amplification, or protein detection. Hybridization methods, in various embodiments, include allele-specific oligonucleotide (ASO) hybridization, hybrid capture assays, hybridization using locked nucleic acid (LNA) probes, reverse dot blot assays, southern blot hybridization, northern blot hybridization, and hybridization using DNA probe arrays.

In some aspects, treatment decisions may be influenced by levels of S6 phosphorylation in subject(s). For example, in some aspects, combinations of an SRC inhibitor and an S6K/AKT inhibitor may be administered to subjects with elevated levels of phosphorylated S6 in comparison to a reference level of phosphorylated S6. The level of phosphorylated S6 may be determined using any method generally known to those of ordinary skill in the art. For example, the level of phosphorylated S6 may be determined using western blot, antibody detection, a kinase activity assay, flow cytometry, immunocytochemistry, immunohistochemistry, mass spectrometry, or multi-analyte profiling.

Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological effect. For example, an effective amount may lead to achieving the desired effect in a subject, such as a reduction in a symptom associated with a condition associated with IDH1/2 mutation(s). Effective amounts are often administered for research purposes. Effective amounts disclosed herein can cause a statistically-significant effect in an animal model or in vitro assay relevant to the assessment of a IDH1/2 mutation(s)-associated condition's development, progression, and/or resolution.

A “prophylactic treatment” includes a treatment administered to a subject who does not display signs or symptoms of a condition associated with IDH1/2 mutation(s) or displays only early signs or symptoms of the condition such that treatment is administered for the purpose of diminishing or decreasing the risk of developing the condition further. Thus, a prophylactic treatment functions as a preventative treatment against a condition associated with IDH1/2 mutation(s). In particular embodiments, prophylactic treatments reduce, delay, or prevent the worsening of the condition.

A “therapeutic treatment” includes a treatment administered to a subject who displays symptoms or signs of a condition associated with IDH1/2 mutation(s) and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment, or therapeutic treatment are not mutually exclusive, and in particular embodiments, administered dosages may accomplish more than one treatment effect.

In particular embodiments, active compounds disclosed herein can be administered together or sequentially to a subject. This may mean that the compounds are administered in a combinatorial formulation or sequentially at the same or different times. In some aspects, the active compounds may be administered within a clinically relevant time window. For example the SRC inhibitor and the S6K/AKT inhibitor may be taken a certain amount of time apart. For example, one may be taken in the morning and the other may be taken in the evening, or they may be taken 0, 1, 2, 3, 4, 5, 6, 7, 8, or more hours or days apart, or one may be taken more than once a day and the other taken only once a day.

In particular embodiments, active compounds disclosed herein can be administered for a clinically relevant time period. For example, active compounds may be administered one or more times a day, week, or month for a period of time. For example, in some aspects, the active compounds may be administered for 9 weeks. In certain embodiments, the period of time is 6 months, one year, two years, five years, ten years, 15 years, 20 years, or the lifetime of the patient. In some aspects, the active compounds disclosed herein may be administered for a specific time period and then patient response may be evaluated. For example, the active compounds may be administered episodically for periods of nine weeks and then re-evaluated.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

In particular embodiments, the condition associated with IDH1/2 mutation(s) is a cancer and therapeutically effective amounts provide anti-cancer effects. Cancers that can be treated with the compositions and methods disclosed herein include ICC, oligodendrogliomas (e.g., anaplastic oligodendroglioma), astrocytomas (e.g., anaplastic astrocytoma and diffuse astrocytoma), glioblastomas (e.g., conventional glioblastoma multiforme), leukemias (e.g., acute myeloid leukemia), adenocarcinomas (e.g., lung adenocarcinoma, colon adenocarcinoma, endometrial endometrioid adenocarcinoma, high grade ovarian serous adenocarcinoma, and pancreatic adenocarcinoma), gliomas (e.g., diffuse glioma and high-grade glioma), melanomas (e.g., cutaneous melanoma), oligoastrocytomas (e.g., anaplastic oligoastrocytoma), invasive breast carcinomas, invasive ductal carcinomas, oligodendrogliomas, and myelodysplastic syndromes.

Anti-cancer effects can include a decrease in the number of cancer cells, a decrease in the number of metastases, a decrease in tumor volume, an increase in life expectancy, induced chemo- or radiosensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

A “tumor” can be liquid or solid depending on the cell origin. A solid tumor is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease and can be considered a solid tumor or liquid tumor in the art depending on the cell origin. Tumors show partial or complete lack of structural organization and functional coordination with the normal tissue, and usually form a distinct mass of tissue, which may be benign, pre-malignant or malignant. Liquid tumors refer to the total mass of circulating neoplastic cells, for examples in hematopoietic malignancies such as leukemia.

In particular embodiments, IDH1/2 mutation-related conditions that can be treated by an SRC inhibitor and an S6K/AKT inhibitor include conditions associated with a level of phosphorylated S6 (pS6) above a reference level of pS6 in cells expressing wildtype IDH.

In particular embodiments, useful doses of active ingredients, individually or in combination, can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

In particular embodiments, useful doses of active ingredients, individually or in combination, can include 1 μCi/kg, 15 μCi/kg, 30 μCi/kg, 50 μCi/kg, 55 μCi/kg, 70 μCi/kg, 90 μCi/kg, 150 μCi/kg, 350 μCi/kg, 500 μCi/kg, 750 μCi/kg, or 1000 μCi/kg. In particular embodiments, a dose includes up to 500 μCi/kg.

In particular embodiments, useful doses of SRC inhibitors disclosed herein include 20-40 kg/mg and useful doses of S6K/AKT inhibitors disclosed herein include 5-15 mg/kg. In particular embodiments, useful doses of SRC inhibitors disclosed herein include 25-23 kg/mg and useful doses of S6K/AKT inhibitors disclosed herein include 8-12 mg/kg. In particular embodiments, useful doses of SRC inhibitors disclosed herein include 30 kg/mg and useful doses of S6K/AKT inhibitors disclosed herein include 10 mg/kg.

(iii) Reference levels. Obtained values for parameters associated with a subject described herein (e.g., a level of phosphorylated S6) can be compared to a reference level derived from a control population. In some examples, this comparison can indicate whether a therapy described herein is appropriate for a subject in need thereof (e.g., whether the therapy may be effective for the subject in need thereof). Reference levels can be obtained from one or more relevant datasets from a control population. A “dataset” as used herein is a set of numerical values resulting from evaluation of a sample (or population of samples) under a desired condition. The values of the dataset can be obtained, for example, by experimentally obtaining measures from a sample and constructing a dataset from these measurements. As is understood by one of ordinary skill in the art, the reference level can be based on, for instance, any mathematical or statistical formula useful and known in the art for arriving at a meaningful aggregate reference level from a collection of individual data points (e.g., mean, median, median of the mean, etc.). Alternatively, a reference level or dataset to create a reference level can be obtained from a service provider such as a laboratory, or from a database or a server on which the dataset has been stored.

A reference level from a dataset can be derived from previous measures derived from a control population. A “control population” is any grouping of subjects or samples of like specified characteristics. The grouping could be according to, for example, clinical parameters, clinical assessments, therapeutic regimens, disease status, severity of condition, etc. In particular embodiments, the grouping is based on age range (e.g., 60-65 years) and cancer status. In particular embodiments, a normal control population includes individuals that are age-matched to a test subject and do not have cancer. In particular embodiments, age-matched includes, e.g., 0-10 years old; 30-40 years old, 60-65 years old, 70-85 years old, etc., as is clinically relevant under the circumstances. In particular embodiments, a control population can include those that have cancer without an IDH1/2 mutation. In particular embodiments, a control population can include those that have not been administered a therapeutically effective amount of a formulation and/or a composition.

In particular embodiments, the relevant reference level for values of a particular parameter associated with a subject in need thereof described herein is obtained based on the value of a particular corresponding parameter associated with a subjects in a control population to determine whether a therapy disclosed herein may be therapeutically effective for the subject in need thereof.

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular data point, where the data point is the result of random chance alone. A result is often considered significant (not random chance) at a p-value less than or equal to 0.05. In particular embodiments, a sample value is “comparable to” a reference level derived from a normal control population if the sample value and the reference level are not statistically significantly different.

In certain examples, reference levels can be used to define thresholds. For example, a threshold could be 10%, 20%, 30%, 40%, or 50% higher than a reference level. In certain examples, a threshold may be 5% to 300% greater than a reference level, 5% to 200% greater than a reference level, 5% to 100% than a reference level, or 10%-75% greater than a reference level.

If a subject meets or exceeds a threshold, they can be administered a combination treatment as disclosed herein. If a subject meets or exceeds the threshold, they can be enrolled in a clinical trial evaluating combination treatments as disclosed herein.

### (iv) Exemplary Embodiments

1. A method of treating a subject having a condition associated with IDH1/2 mutation(s), the method including administering to the subject
a therapeutically effective amount of an SRC inhibitor; and
a therapeutically effective amount of an S6K/AKT inhibitor thereby treating the subject having the condition associated with IDH1/2 mutation(s).
   2. The method of embodiment 1, wherein the SRC inhibitor includes dasatinib, saracatinib, bosutinib, NXP900, KX01, KX2-391, PP1, or PP2.
   3. The method of embodiment 1, wherein the SRC inhibitor includes dasatinib or saracatinib.
   4. The method of any of embodiments 1-3, wherein the S6K/AKT inhibitor includes M2698, pyrazolopyrimidines, LY2780301, LY2584702, GNE-477, paxalisib, pyrvinium pamoate, PF-4708671, or MSC2363318A.
   5. The method of any of embodiments 1-4, wherein the SRC inhibitor includes dasatinib and the S6K/AKT inhibitor includes M2698.
   6. The method of any of embodiments 1-5, wherein the condition associated with IDH1/2 mutation(s) includes cholangiocarcinoma, oligodendroglioma, astrocytoma, leukemia, adenocarcinoma, glioma, melanoma, oligoastrocytoma, breast carcinoma, or myelodysplastic syndrome.
   7. The method of embodiment 6, wherein the cholangiocarcinoma includes intrahepatic cholangiocarcinoma.
   8. The method of any of embodiments 1-7, wherein the administering is intravenous, intradermal, intraarterial, intranodal, intravesicular, intrathecal, intraperitoneal, intraparenteral, intranasal, intralesional, intramuscular, oral, intrapulmonary, subcutaneous, or sublingual.
   9. The method of any of embodiments 1-8, wherein the SRC inhibitor and the S6K/AKT inhibitor are administered together.
   10. The method of any of embodiments 1-9, further including assessing a phosphorylation level of S6 in a sample derived from the subject.
   11. The method of any of embodiments 1-10, wherein a phosphorylation level of S6 in a sample derived from the subject exceeded a threshold.
   12. The method of any of embodiments 1-11, further including identifying IDH1 and/or IDH2 mutation(s) in a sample derived from the subject.
   13. The method of any of embodiments 1-12, wherein the subject has IDH1/2 mutation(s) comprising an IDH1 mutation at residue 132.
   14. The method of any of embodiments 1-13, wherein the subject has IDH1/2 mutation(s) comprising an IDH2 mutation at residue 140
   15. The method of any of embodiments 1-14, wherein the subject has IDH1/2 mutation(s) comprising an IDH2 mutation at residue 172.
   16. The method of any of embodiments 1-15, wherein the subject has IDH1/2 mutation(s) comprising an IDH1 mutation at residue 132 and an IDH2 mutation at residue 140.
   17. The method of any of embodiments 1-16, wherein the subject has IDH1/2 mutation(s) comprising an IDH1 mutation at residue 132 and an IDH2 mutation at residue 172.
   18. The method of any of embodiments 1-17, wherein the subject has IDH1/2 mutation(s) comprising an IDH2 mutation at residue 140 and an IDH2 mutation at residue 172.
   19. The method of any of embodiments 1-18, further including diagnosing the subject with the condition associated with IDH1/2 mutation(s).
   20. A method for evaluating a subject having a condition associated with IDH1/2 mutation(s) for enrollment in a clinical trial, the method including
assessing a phosphorylation level of S6 in a sample derived from the subject; and
enrolling the subject in the clinical trial if the phosphorylation level meets or exceeds a threshold.
   21. The method of embodiment 20, wherein the condition associated with IDH1/2 mutation(s) includes cholangiocarcinoma, oligodendroglioma, astrocytoma, leukemia, adenocarcinoma, glioma, melanoma, oligoastrocytoma, breast carcinoma, or myelodysplastic syndrome.
   22. The method of embodiment 21, wherein the cholangiocarcinoma includes intrahepatic cholangiocarcinoma.
   23. The method of any of embodiments 20-22, wherein the clinical trial includes a protocol directing administration of an SRC inhibitor and an S6K/AKT inhibitor.
   24. The method of embodiment 23, wherein the SRC inhibitor includes dasatinib, saracatinib, bosutinib, NXP900, KX01, KX2-391, PP1, or PP2.
   25. The method of embodiment 23, wherein the SRC inhibitor includes dasatinib or saracatinib.
   26. The method of any of embodiments 17-19, wherein the S6K/AKT inhibitor includes M2698, pyrazolopyrimidines, LY2780301, LY2584702, GNE-477, paxalisib, pyrvinium pamoate, PF-4708671, or MSC2363318A.
   27. The method of any of embodiments 23-26, wherein the SRC inhibitor includes dasatinib and the S6K/AKT inhibitor includes M2698.
   28. The method of any of embodiments 20-27, wherein the subject has IDH1/2 mutation(s) comprising an IDH1 mutation at residue 132.
   29. The method of any of embodiments 20-28, wherein the subject has IDH1/2 mutation(s) comprising an IDH2 mutation at residue 140
   30. The method of any of embodiments 20-29, wherein the subject has IDH1/2 mutation(s) comprising an IDH2 mutation at residue 172.
   31. The method of any of embodiments 20-30, wherein the subject has IDH1/2 mutation(s) comprising an IDH1 mutation at residue 132 and an IDH2 mutation at residue 140.
   32. The method of any of embodiments 20-31, wherein the subject has IDH1/2 mutation(s) comprising an IDH1 mutation at residue 132 and an IDH2 mutation at residue 172.
   33. The method of any of embodiments 20-32, wherein the subject has IDH1/2 mutation(s) comprising an IDH2 mutation at residue 140 and an IDH2 mutation at residue 172.
   34. A composition including an SRC inhibitor, an S6K/AKT inhibitor, and a pharmaceutically acceptable carrier.
   35. The composition of embodiment 34, wherein the SRC inhibitor includes dasatinib, saracatinib, bosutinib, NXP900, KX01, KX2-391, PP1, or PP2.
   36. The composition of embodiment 34, wherein the SRC inhibitor includes dasatinib or saracatinib.
   37. The composition of any of embodiments 34-36, wherein the S6K/AKT inhibitor includes M2698, pyrazolopyrimidines, LY2780301, LY2584702, GNE-477, paxalisib, pyrvinium pamoate, PF-4708671, or MSC2363318A.
   38. The composition of any of embodiments 34-37, wherein the SRC inhibitor includes dasatinib and the S6K/AKT inhibitor includes M2698.
   39. A kit including an SRC inhibitor and an S6K/AKT inhibitor.
   40. The kit of embodiment 39, further including a pharmaceutically acceptable carrier.
   41. The kit of embodiment 39 or 40, wherein the SRC inhibitor includes dasatinib, saracatinib, bosutinib, NXP900, KX01, KX2-391, PP1, or PP2.
   42. The kit of any of embodiments 39-41, wherein the SRC inhibitor includes dasatinib or saracatinib.
   43. The kit of any of embodiments 39-42, wherein the S6K/AKT inhibitor includes M2698, pyrazolopyrimidines, LY2780301, LY2584702, GNE-477, paxalisib, pyrvinium pamoate, PF-4708671, or MSC2363318A.
   44. The kit of any of embodiments 39-43, wherein the SRC inhibitor includes dasatinib and the S6K/AKT inhibitor includes M2698.
   45. The kit of any of embodiments 39-44, further including instructions to administer the SRC inhibitor and the S6K/AKT inhibitor to a subject having a condition associated with IDH1/2 mutation(s).
   46. The kit of embodiment 45, wherein the condition associated with IDH1/2 mutation(s) includes cholangiocarcinoma, oligodendroglioma, astrocytoma, leukemia, adenocarcinoma, glioma, melanoma, oligoastrocytoma, breast carcinoma, or myelodysplastic syndrome.
   47. The kit of embodiment 46, wherein the cholangiocarcinoma includes intrahepatic cholangiocarcinoma.
   48. The kit of any of embodiments 39-47, further including instructions to administer the SRC inhibitor and the S6K/AKT inhibitor to a subject having an S6 phosphorylation level that exceeds a threshold.
   49. A method of treating a subject in need thereof, including:
administering to the subject in need:
a therapeutically effective amount of an SRC inhibitor; and
a therapeutically effective amount of an S6K/AKT inhibitor.
   50. The method of embodiment 49, wherein the SRC inhibitor includes one or more of: dasatinib, saracatinib, bosutinib, NXP900, KX01, KX2-391, PP1, or PP2.
   51. The method of embodiments 49 or 50, wherein the SRC inhibitor includes dasatinib or saracatinib.
   52. The method of any of embodiments 49-51, wherein the S6K/AKT inhibitor includes one or more of: M2698, pyrazolopyrimidines, LY2780301, LY2584702, GNE-477, paxalisib, pyrvinium pamoate, PF-4708671, or MSC2363318A.
   53. The method of any of embodiments 49-52, wherein the therapeutically effective amount provides a therapeutic treatment against a condition associated with IDH1/2 mutation(s).
   54. The method of embodiment 53, wherein the condition associated with IDH1/2 mutation(s) includes at least one of: a cholangiocarcinoma, an oligodendroglioma, an astrocytoma, a leukemia, an adenocarcinoma, a glioma, a melanoma, an oligoastrocytoma, a breast carcinoma, or a myelodysplastic syndrome.
   55. The method of embodiment 53 or 54, wherein the condition associated with IDH1/2 mutation(s) includes intrahepatic cholangiocarcinoma.
   56. The method of any of embodiments 49-55, wherein the therapeutically effective amount of the SRC inhibitor includes a dosage of 30 mg/kg.
   57. The method of any of embodiments 49-56, wherein the therapeutically effective amount of the S6K/AKT inhibitor includes a dosage of 10 mg/kg.
   58. The method of any of embodiments 49-57, wherein the administering is through intravenous, intradermal, intraarterial, intranodal, intravesicular, intrathecal, intraperitoneal, intraparenteral, intranasal, intralesional, intramuscular, oral, intrapulmonary, subcutaneous, or sublingual administering.
   59. The method of any of embodiments 49-58, wherein the therapeutically effective amount of the SRC inhibitor and the therapeutically effective amount of the S6K/AKT inhibitor are administered together.
   60. The method of any of embodiments 49-58, wherein the therapeutically effective amount of the SRC inhibitor is administered within a clinically relevant time window of the therapeutically effective amount of an S6K/AKT inhibitor.
   61. The method of any of embodiments 49-59, wherein the subject in need thereof has a level of phosphorylated S6 (pS6) 5% to 300% higher than a reference level, wherein the reference level includes a pS6 level obtained from:
a biological sample of an individual who does not have cancer,
the subject at an earlier point in time, or
an individual with cancer without an IDH1/2 mutation.
   62. The method of embodiment 61, wherein the level of pS6 in the subject includes a range of 10 to 75% higher than the reference level.
   63. The method of any of embodiments 49-62, further including:
identifying IDH1 and/or IDH2 mutation(s) in the subject.
   64. The method of embodiment 63, wherein the identifying identifies IDH1/2 mutation(s) comprising an IDH1 mutation at residue 132.
   65. The method of embodiment 63 or 64, wherein the identifying identifies IDH1/2 mutation(s) comprising an IDH2 mutation at residue 140
   66. The method of any of embodiments 63-65, wherein the identifying identifies IDH1/2 mutation(s) comprising an IDH2 mutation at residue 172.
   67. The method of any of embodiments 63-66, wherein the identifying identifies IDH1/2 mutation(s) comprising an IDH1 mutation at residue 132 and an IDH2 mutation at residue 140.
   68. The method of any of embodiments 63-67, wherein the identifying identifies IDH1/2 mutation(s) comprising an IDH1 mutation at residue 132 and an IDH2 mutation at residue 172.
   69. The method of any of embodiments 63-68, wherein the identifying identifies IDH1/2 mutation(s) comprising an IDH2 mutation at residue 140 and an IDH2 mutation at residue 172.
   70. The method of any of embodiment 63-69, wherein identifying one or more IDH1 and/or IDH2 mutation(s) in the subject includes performing on a sample comprising cells of the subject one or more of: direct nucleic acid sequencing, hybridization methods, restriction enzyme digestion, polymerase chain reaction (PCT) amplification, or protein detection.
   71. A method of treating a subject in need thereof, including:
measuring a level of phosphorylated S6 (pS6) in a sample from the subject;
determining if the level of pS6 in the subject is higher than a reference pS6 level; and
when the level of pS6 in the subject is higher than the reference pS6 level administering to the subject:
   a therapeutically effective amount of an SRC inhibitor; and
   a therapeutically effective amount of an S6K/AKT inhibitor.
      72. The method of embodiment 71, wherein the level of pS6 in the subject is determined via western blot, antibody detection, a kinase activity assay, flow cytometry, immunocytochemistry, immunohistochemistry, mass spectrometry, or multi-analyte profiling.
      73. The method of embodiment 71 or 72, wherein the level of pS6 in the subject includes a range of 5 to 300% higher than a reference level, wherein the reference level comprises a pS6 level obtained from:
a biological sample of an individual who does not have cancer,
the subject at an earlier point in time, or
an individual with cancer without an IDH1/2 mutation.
   74. The method of any of embodiments 71-73, wherein the level of pS6 (pS6) in the subject includes a range of 10 to 75% higher than the reference level.
   75. A composition, comprising:
an SRC inhibitor; and
an S6K/AKT inhibitor.
   76. The composition of embodiment 75, wherein the SRC inhibitor comprises dasatinib, saracatinib, bosutinib, NXP900, KX01, KX2-391, PP1, or PP2.
   77. The composition of embodiment 75 or 76, wherein the SRC inhibitor includes dasatinib or saracatinib.
   78. The composition of any of embodiments 75-77, wherein the S6K/AKT inhibitor includes M2698, pyrazolopyrimidines, LY2780301, LY2584702, GNE-477, paxalisib, pyrvinium pamoate, PF-4708671, or MSC2363318A.
   79. The composition of any of embodiments 75-78, further including a pharmaceutically acceptable carrier.
   80. The composition of any of embodiment 75-79, wherein the SRC inhibitor is formulated with a first pharmaceutically acceptable carrier and wherein the S6K/AKT inhibitor is formulated with a second pharmaceutically acceptable carrier.

(v) Experimental Examples. The current disclosure describes identification of a new SRC regulated survival mechanism in IDHm ICC cells. While not wishing to be bound, inhibition of SRC by dasatinib treatment potently inhibits p70 S6 kinase (S6K) and Ribosomal protein S6 (S6), members of the mTORC1 pathway, exclusively in IDHm cells and leads to significant reductions in cell size and de novo protein synthesis. In this experimental example, there were no effects on other upstream (AKT, mTOR) components of the mTOR pathway.

Using an unbiased phosphoproteomic screen, a scaffolding molecule, Membrane Associated Guanylate Kinase, WW And PDZ Domain Containing 1 (MAGI1) was identified as a novel SRC substrate in IDHm ICC. Upon SRC inhibition, MAGI1 forms a tumor suppressive complex with Protein Phosphatase 2A (PP2A) facilitating S6K dephosphorylation and suppression of S6K/S6 signaling. Inhibition of mutant IDH was found to partially rescue S6K/S6 signaling and dasatinib-induced cytotoxicity. Together, this study characterized the molecular mechanism underlying dasatinib hypersensitivity and revealed a suppressive MAGI1-PP2A signaling complex that functions to inactivate S6K/S6 in IDHm ICC.

Materials and Methods. Study design. The goals of this study were to dissect the underlying mechanism by which IDHm ICC cells are sensitive to SRC inhibition and to evaluate combination treatments to overcome resistance for potential clinical applications. These objectives were accomplished by (i) measuring signaling responses to drug treatments and gene silencing by analysis of survival pathways; (ii) assessing cellular changes by functional assays such as flow cytometry for apoptosis, cell size, and puromycin uptake for translation; (iii) identifying SRC substrates by unbiased phosphoproteomic screen and physical interaction of SRC, MAGI1, PP2A, and S6K signaling complex by co-IPs; (iv) evaluating marker for dasatinib resistance and the effect of combining dasatinib with S6K inhibitor M2698 in overcoming treatment resistance using cell line, PDO (in vitro), and PDX (in vivo) models. For in vitro experiments, the maximum number of available human IDHm cell lines and successfully derived PDO lines were used. For e experiments, sample size and treatment regimen were determined on the basis of published literature and past experience. Mice were randomized into treatment arms. Investigators were blinded to the treatment effect, and a blinded pathologist performed all histological analyses for murine studies. All in vitro experiments were performed with a minimum of two replicates or as indicated.

Cell Lines. Cell lines were obtained from the Riken Bioresource Center (RBE, HuCCT1), the Korean Cell Line Bank (SNU-1079), or were derived as previously described (ICC2, ICC5) (S. Saha, et al. Cancer Discov 6, 727-739 (2016)). CC-LP-1 was a kind gift from Dr. P.J. Bosma (Academic Medical Center, Amsterdam, the Netherlands). RBE IDH1 S132R WT Knock-in (KI) pool cells were generated from parental RBE cells with IDH1 R132S by Synthego using guide RNA sequence: TCATAGGTCGTCATGCTTAT (SEQ ID NO. 1).

RBE KI cell lines were then generated from pooled cells by limiting dilution. Each KI clone was sequenced to confirm the conversion of encoded amino acid from Serine to Arginine. Cell lines were grown at 37° C. under 5% CO₂ in their required growth medium (Gibco) supplemented with 10% fetal bovine serum and 1% penicillin and streptomycin. Cells were passaged by trypsinization.

Patient-derived organoids (PDO) culturing and drug treatments. Human organoids were derived and cultured according to the methods detailed previously (Boj, et al., Cell 160, 324-338 (2015)). Human ICC tissue from surgical resected samples were obtained from Dr. Raymond Yeung (Department of Surgery, University of Washington), IRB #00001852. Fresh tumor chunks were minced and digested with collagenase II (5 mg/ml, Gibco), DNAse I (10 μg/ml, Sigma) and Y-27632 Rho Kinase inhibitor (10.5 μM, Sigma) in human feeding media in a rotating incubator set at 37° C. and 35 rpm rotation for 3 rounds of 15 minutes each. Cells isolated from human tissue were embedded in Matrigel (Corning) and cultured on a 24-well plate in human complete media. Media were changed every 2-4 days. For drug treatment, media were changed to human complete medium containing drug at the desired concentration. After 16 hours of drug treatment, organoids were harvested by resuspending domes in Cell Recovery Solution (Corning) and washed with cold PBS before snap-freezing the pellets for subsequent protein isolation and western blotting as described below.

Proliferation Assays. Cells were plated in 96-well plates (1,000 cells/well) in culture medium. After 24 hours, increasing doses of either dasatinib, AG-120, or DMSO control were added. The cells were allowed to grow 5-7 days until control wells were confluent. Viable cells were quantified by adding MTT (M-6494; ThermoFisher Scientific) at a final concentration of 1 mg/mL for 3 hours at 37° C. 100 μL/well of DMSO was added to solubilize formazan crystals. Absorbance was read at 490 nm and normalized to DMSO control. For each experimental condition, duplicate wells were seeded, and data is represented as mean±SEM among three independent experiments.

Annexin V Apoptosis Assay. Cells were washed twice with cold PBS and then resuspended in 1× Binding Buffer at a concentration of 1×106 cells/ml. 1×105 cells were transferred to a 5 ml culture tube, and 5 μl of FITC Annexin V and 5 μl PI (FITC Annexin V Apoptosis Detection Kit, BD Pharmingen) were added to the cells and incubated for 15 min at RT (25° C.). 400 μl of 1× Binding Buffer to each tube and Annexin V positive cells and PI positive cells were analyzed by flow cytometry. Data are shown as mean±SEM between triplicates and are representative of three independent experiments.

Co-immunoprecipitation. Cells were washed twice with 1× PBS and lysed with lysis buffer (0.3% CHAPS, 25 mM HEPES pH 7.9, 2 mM EGTA, 2.5 mM MgCl2) supplemented with protease and phosphatase inhibitors. Lysates were sonicated using a QSONICA Q800R3 sonicator for 5 mins (30 seconds ON, 30s OFF, 20% AMP; samples in 4° C. water bath throughout sonication cycles). The lysate was centrifuged at 15,000 rpm for 10 minutes at 4° C., and protein concentration of the supernatant was measured using a BCA protein assay kit (Pierce). For each sample, 50 μL of Dynabeads protein G beads (Invitrogen) was incubated with 1 μg of antibody (Santa Cruz Biotechnology: c-Myc (sc-40), mouse IgG (sc-3877); Sigma: FLAG (F1804)) for 10 minutes while rotating. Protein lysates were adjusted to 1 mg/ml and 500 μL of lysate was incubated with antibody-bound Dynabeads for 10 minutes while rotating. 5 μL of lysate was kept as input. Beads were collected and washed with PBS three times and immunoprecipitates were collected by boiling beads in 50 μL of sample buffer. Immunoprecipitates (50 μL) were then loaded on a 4-20% gradient polyacrylamide gel with SDS (Biorad) for western blot analysis as described previously. Results are representative of two independent experiments.

Protein Isolation and Western Blot. Protein lysates were prepared by lysing cells directly in NP-40 buffer supplemented with a protease inhibitor cocktail (Complete EDTA-free, Roche Applied Science), 5 μM and phosphatase inhibitors (Phosphatase Inhibitor Cocktail Sets I and II, Calbiochem). Cells suspended in lysis buffer were sonicated for 10 seconds of active sonication, followed by 20 seconds of rest for 3 cycles at 20% amplitude. The lysate was then centrifuged at 14,000 rpm for 10 mins at 4° C. and the supernatant was harvested. A BCA protein assay kit (Pierce) was used to measure and normalize protein concentration. 30 μg of the cell lysate was run on a 4-20% gradient polyacrylamide gel with SDS (Biorad) and electroblotted onto polyvinylidene difluoride membranes (PVDF) (Millipore). Membranes were blocked in TBS-T with 5% non-fat milk and 0.1% Tween and probed with primary antibodies (Table 1). Horseradish-peroxidase-conjugated secondary antibodies (Vector Biolaboratories) were used to detect membrane-bound proteins. Blots were developed using Clarity Max Western ECL Blotting Substrate (Biorad). Signaling experiments are representative of at least two independent experiments.

TABLE 1
List of Antibodies.

| Protein Name | Source | Catalog Number |
|---|---|---|
| Phospho-SRC Y416 | CST | 6943S |
| Phospho-SRC Y527 | CST | 2105 |
| Src | CST | 2123S |
| Phospho-p70 S6 Kinase T389 | CST | 9234S |
| p70 S6 Kinase | CST | 2708S |
| Phospho-S6 S235/236 | CST | 4858S |
| S6 | CST | 2217S |
| Phospho-4E-BP1 T37/46 | CST | 2855T |
| 4E-BP1 | CST | 9644T |
| Phospho PP2A Y307 | R&D Systems | AF3989 |
| PP2A | CST | 2038S |
| MAGI1c | Santa Cruz | sc-100326 |
| Phospho-AKT T308 | CST | 13038S |
| Phospho-AKT S473 | CST | 9271S |
| AKT | CST | 9272S |
| Phospho-mTOR S2481 | CST | 2974S |
| mTOR | CST | 2972S |
| Phospho-ULK1 S757 | CST | 14202T |
| Total ULK1 | CST | 8054S |
| Phospho-STAT3 | CST | 9134S |
| STAT3 | CST | 9139S |
| Phospho-ERK 1/2 Y202/Y204 | CST | 2708S |
| ERK 1/2 | CST | 4695S |
| B-Tubulin | CST | 2146S |
| B-Actin | Sigma | A5316 |
| Puromycin | Sigma | MABE343 |
| MYC | Santa Cruz | sc-40 |
| GFP | CST | 2555S |
| HA tag | abcam | ab9110 |
| FLAG | Sigma | F1804 |
| PARP | CST | 9542 |
| Cleaved caspase 3 | CST | 9664 |
| anti-Rabbit IgG (H + L) Alexa Fluor 488 | Thermo Fisher | A-11034 |

pMAGI Y373 antisera. Rabbit anti-pY373 MAGI antibodies against synthetic peptide CGEDPV (pY) GIY-amide were made and linked, via the N-terminal cysteine, to maleimide-activated KLH (Pierce Biotechnology, Rockford, IL) according to manufacturer's instructions. Polyclonal antibodies were produced in rabbits by custom commercial preparation (ProSci Inc., Poway, CA; 13 week schedule). Phosphospecific antibody was affinity purified according to published methods (Lampe, et al., J Cell Sci 119, 3435-3442 (2006)). Cell Size Flow Cytometry. Cell culture plates were washed with PBS to remove floating cells. Cell plates were then dissociated into single cells using Trypsin-EDTA 0.25% (Gibco) for no longer than 2 minutes before being quenched with culture media. Cells were gently pelleted and washed with cold PBS. Cells were then fixed with 70% ethanol for at least 24 hrs at 4° C. before flow cytometry analysis. Average cell size was determined by median FSC-A counts. Data are shown as mean±SEM between triplicates and are representative of three independent experiments.

Puromycin Uptake Assays. Directly prior to collection, cells were treated with 1 ug/mL Puromycin and incubated at 37° C. for 30 minutes. Cell plates were washed immediately with ice-cold PBS after the 30 min incubation. Protein isolation and western blotting was performed as previously described. Results are representative of two independent experiments.

Phosphoproteomics. Protein Digestion and TMT Labeling. Cell pellets were re-suspended in cell lysis buffer (75 mM NaCl, 50 mM HEPES [pH 8.5], 10 mM sodium pyrophosphate, 10 mM NaF, 10 mM β-glycerophosphate, 10 mM sodium orthovanadate, 1 mM PMSF, 3% SDS, and complete mammalian protease inhibitor tablet [Roche]) by passing the suspension through a 21-gauge needle 20 times. Dithiothreitol (DTT) was used to reduce disulfide bonds and free thiols were alkylated with iodoacetamide (IAA) as described previously (Ting, et al., Nature methods 8, 937-940 (2011)). Reduced and alkylated proteins were then precipitated with following the methanol/chloroform method precipitation as described previously (Lyons, et al., Sci Signal 11, (2018)). Precipitated proteins were reconstituted in 300 μL of 1 M urea in 50 mM HEPES, pH 8.5. Vortexing and sonication were used to aid solubility. Proteins were then digested in a two-step process, first with 3 μg endoproteinase Lys-C (Wako) for 17 hours at room temperature (RT) and then with 3 μg sequencing-grade trypsin (Promega) for 6 hours at 37° C. The digest was acidified with trifluoroacetic acid (TFA). Peptides were desalted over Sep-Pak C18 solid-phase extraction (SPE) cartridges (Waters). The peptide concentration was determined using a BCA assay (Thermo Scientific) and a maximum of 50 μg of peptides were aliquoted, then dried under vacuum and stored at −80° C. prior to labeling with TMT reagents. Peptides were labeled with 10-plex tandem mass tag (TMT) reagents (Thermo Scientific). TMT reagents were suspended in dry acetonitrile (ACN) at a concentration of 20 μg/μL. Dried peptides were re-suspended in 30% dry ACN in 200 mM HEPES, pH 8.5, and 5 μL of the appropriate TMT reagent were added to the sample, which was incubated at RT for one hour. The reaction was then quenched by adding 6 μl of 5% (w/v) hydroxylamine in 200 mM HEPES (pH 8.5) and incubated for 15 min at RT. The solutions were acidified by adding 50 μl of 1% TFA, combined into one sample, and desalted. If the number of samples exceeded ten, samples were split across two TMT sets and a bridge sample generated by pooling a part of all sample was added to each TMT set (Lapek, Jr., et al., Nat Biotechnol 35, 983-989 (2017)).

Basic pH Reversed-Phase Liquid Chromatography (bRPLC) Sample Fractionation. Basic pH reversed-phase liquid chromatography (bRPLC) was used to perform sample fractionation with concatenated fraction combining. Briefly, samples were re-suspended in 5% formic acid (FA)/5% ACN and separated over a 4.6 mm×250 mm ZORBAX Extend C18 column (5 μm, 80 Å, Agilent Technologies) on an Agilent 1260 HPLC system outfitted with a fraction collector, degasser and variable wavelength detector. A two-buffer system (Buffer A: 5% ACN, 10 mM ammonium bicarbonate; Buffer B: 90% ACN, 10 mM ammonium bicarbonate) was used for separation, with a 20-35% gradient of Buffer B over 60 minutes at a flowrate of 0.5 mL/minute. A total of 96 fractions were collected, which were combined in a total of 24 fractions. The combined fractions were dried under vacuum, re-constituted with 8 μL of 5% FA/5% ACN, 3 μL of which were analyzed by LC-MS2/MS3.

Phosphopeptide enrichment. Peptides were subjected to enrichment for phosphopeptides enrichment using a 4:1 ratio of titanium dioxide beads to peptide (w/w) (Lyons, supra; Kreuzer, et al., Methods Enzymol 626, 41-65 (2019)). Peptides were resuspended in 2 M lactic acid in 50% ACN and added to 1.8 mg of titanium dioxide beads. The mixture was shaken gently for 1 hour. Beads were collected by centrifugation and washed 3 times with 2 M lactic acid in 50% ACN and 3 times with 50% ACN/0.1% TFA. Phosphopeptides were eluted with 2×200 μL of 50 mM KH2PO4, pH 10, and acidified with 1% TFA. Eluted phosphopeptides were desalted, lyophilized, and labeled with 2 μL of 10-plex TMT reagents 127n-130c as described above. The combined sample was enriched for phosphotyrosine-containing peptides using phosphotyrosine antibody-conjugated beads (Cell Signaling Technology) following the protocol provided by the manufacturer. Unbound peptides (phosphoserine and phosphothreonine peptides) were desalted, lyophilized, and fractionated by bRPLC using a gradient of 5-28% Buffer B. A total of 96 fractions were collected, and fractions were combined into 12 fractions. Bound peptides (phosphotyrosine peptides) were eluted and desalted. All 13 fractions were re-suspended in 5% ACN/5% formic acid and analyzed on an Orbitrap Fusion mass spectrometer using LC-MS2/MS3 for identification and quantification of the phosphopeptides.

Mass spectrometry data acquisition and analysis. Combined sample fractions were dried, re-suspended in 5% ACN/5% formic acid, and analyzed in 3-hour runs via LC-M2/MS3 on an Orbitrap Fusion mass spectrometer using the Simultaneous Precursor Selection (SPS) supported MS3 method (Lyons, supra; Ting, et al., Nat Methods 8, 937-940 (2017); McAlister, et al., Anal Chem 86, 7150-7158 (2014).; Erickson, et al., Anal Chem 87, 1241-1249 (2015)). Two MS2 spectra were acquired per peptide upon HCD fragmentation and CID fragmentation followed by an SPS-MS3 spectrum on the CID fragment ions (Lyons, supra.; Kreuzer, supra). MS2 spectra were assigned using a SEQUEST-based in-house built proteomics analysis platform (Huttlin, et al., Cell 143, 1174-1189 (2010)) allowing phosphorylation of serine, threonine, and tyrosine residues as a variable modification. The Ascore algorithm was used to evaluate the correct assignment of phosphorylation within the peptide sequence (Beausoleil, et al., Nat Biotechnol 24, 1285-1292 (2006)). Based on the target-decoy database search strategy (Elias and Gygi, Nat Methods 4, 207-214 (2007)) and employing linear discriminant analysis and posterior error histogram sorting, peptide and protein assignments were filtered to false discovery rate (FDR) of <1% (Huttlin, supra). Peptides with sequences that were contained in more than one protein sequence from the UniProt database were assigned to the protein with most matching peptides (Huttlin, supra). TMT reporter ion intensities were extracted as that of the most intense ion within a 0.03 window around the predicted reporter ion intensities in the collected MS3 spectra. Only MS3 with an average signal-to-noise value of larger than 40 per reporter ion as well as with an isolation specificity of larger than 0.75 were considered for quantification (Lyons, supra). A two-step normalization of the protein TMT-intensities was performed by first normalizing the protein intensities over all acquired TMT channels for each protein based on the median average protein intensity calculated for all proteins. To correct for slight mixing errors of the peptide mixture from each sample a median of the normalized intensities was calculated from all protein intensities in each TMT channel and the protein intensities were normalized to the median value of these median intensities. All mass spectrometer RAW files can be accessed through the MassIVE data repository (massive.ucsd.edu) under the accession number MSV000089619.

shRNA Transfection. Viral particles containing Human shSRC #1 (TRCN0000195339) target sequence: 5-CATCCTCAGGAACCAACAATT-3′ (SEQ ID NO: 2); shSRC #2 (TRCN0000199186) target sequence: 5-CTGACTGAGCTCACCACAAAG-3′ (SEQ ID NO: 3) were synthesized using retroviral (pCL-ECO) packaging plasmids with pCMV-VSV-G (Addgene). pLKO.1 shRNA with target sequence 5-GCAAGCTGACCCTGAAGTTCAT-3′ (SEQ ID NO: 4) was used as a negative control. Cells were incubated with virus and 8 μg/mL polybrene (Millipore, #TR-1003-G) for 24 hours and subsequently selected in 2.5 μg/mL puromycin for at least 2 days. Westerns blots were performed as previously described and results are representative of two independent experiments.

siRNA Transfection. Cells were transfected with pooled siRNA targeting MAGI1c (Dharmacon) PPP2CA/B (Dharmacon) or SRC (Dharmacon) at a final concentration of 40 nM using Lipofectamine RNAimax. Cells were harvested 48 to 72 hours after transfection and processed as stated previously. Results are representative of two independent experiments.

Okadaic Acid Rescue. Cells were treated with 100 nM dasatinib or DMSO for 6 hours. Okadaic acid (final concentration 1 μM) was added at the listed time intervals (15, 20, 25, 30 mins) prior to harvest. Protein isolation western blotting were done as previously described. Results are representative of two independent experiments.

Crystal Violet Staining. Cells were washed with cold PBS twice on 6-well plate followed by fixing with ice cold methanol on ice for 10 mins on an orbital shaker. After fixation, cells were stained with 0.5% crystal violet solution in methanol for 10 mins at RT on a shaker and staining solution was then washed off from plate by running water. Results are representative of three independent experiments.

Polar Metabolite Extraction. At the time of sample collection, cell counts and total cell volume was obtained using a Beckman Coulter Counter Multisizer 4. Cells were washed three times in ice-cold blood bank saline (Fisher, cat #23293184) before being scraped in 1 ml of cold 80% HPLC-grade methanol (Sigma cat #646377) in HPLC-grade water (Sigma, cat #270733). Samples were spun at 17,000 g for 5 minutes at 4° C., and aliquots equivalent to 1 μl total cell volume were taken from each sample and lyophilized in a vacuum centrifuge (Labconco Centrivap) and stored at −80° C. until time of analysis. A standard curve of isotopically labelled 2-hydroxyglutarate (Sigma cat #36106) ranging from 100 nM to 1 mM was generated in 80% HPLC grade methanol and measured in parallel.

Liquid Chromatography-Mass Spectrometry (LCMS). Shortly before mass spectrometry analysis, lyophilized samples were resuspended in 100 μl of 80% HPLC grade methanol in HPLC grade water containing 10 uM isotopically labelled 2-hydroxyglutarate (2-HG) and transferred to liquid chromatography-mass spectrometry (LCMS) vials (Thermo Fisher cat #6ESV9-04PP). Metabolite quantitation was performed using a Q Exactive HF-X Hybrid Quadrupole-Orbitrap Mass Spectrometer equipped with an Ion Max API source and H-ESI II probe, coupled to a Vanquish Flex Binary UHPLC system (Thermo Scientific). Mass calibrations were completed at a minimum of every 5 days in both the positive and negative polarity modes using LTQ Velos ESI Calibration Solution (Pierce). Polar Samples were chromatographically separated by injecting a sample volume of 1 μL into a SeQuant ZIC-pHILIC Polymeric column (2.1×150 mm 5 mM, EMD Millipore). The flow rate was set to 150 mL/min, autosampler temperature set to 10° C., and column temperature set to 30° C. Mobile Phase A consisted of 20 mM ammonium carbonate and 0.1% (v/v) ammonium hydroxide, and Mobile Phase B consisted of 100% acetonitrile. The sample was gradient eluted (% B) from the column as follows: 0-20 min; linear gradient from 85% to 20% B; 20-24 min; hold at 20% B; 24-24.5 min; linear gradient from 20% to 85% B; 24.5 min.-end: hold at 85% B until equilibrated with ten column volumes. Mobile Phase was directed into the ion source with the following parameters: sheath gas=45, auxiliary gas=15, sweep gas=2, spray voltage=2.9 kV in the negative mode or 3.5 kV in the positive mode, capillary temperature=300° C., RF level=40%, auxiliary gas heater temperature=325° C. Mass detection was conducted with a resolution of 240,000 in full scan mode, with an AGC target of 3,000,000 and maximum injection time of 250 msec. Metabolites were detected over a mass range of 70-1050 m/z. Quantitation of all metabolites was performed using Tracefinder 4.1 (Thermo Scientific) referencing an in-house metabolite standards library using ≤5 ppm mass error and isotopically labelled standard. After verifying that peak area scaled linearly with 2-HG abundance in the standard curve, 2-HG concentrations were obtained by comparing peak areas of unlabeled 2-HG and 10 μM isotopically labelled 2-HG spike-in.

Immunofluorescence Staining, Imaging and Quantification. Cells were seeded onto 8-chamber slides for 48 hours and treated with DMSO or dasatinib at 50 nM for 6 hours. Cells were fixed by 4% paraformaldehyde and permeabilized by 0.5% Triton X in PBS, followed by blocking with 5% BSA for 30 mins at room temperature. Cells were then incubated with pS6 or S6 primary antibodies at 1:100 in 5% BSA for 2 hours at room temperature. Following washes with PBS, slides were incubated with 1:500 anti-Rabbit IgG (H+L) Alexa Fluor 488 secondary antibody (Thermo Fisher) in 5% BSA for 1 hour, washed with PBS and counterstained and mounted using VECTASHIELD with DAPI (H-1200, Vector Lab). Cells were imaged on confocal microscope (Zeiss LSM 780) and then analyzed using CellProfiler. Images were saved from the DAPI and Alexa Fluorophore 488 channel separately and converted into grayscale to run through a CellProfiler pipeline. The pipeline was created to first identify the primary object of interest as cells from DAPI staining. Once each cell had been designated, the pS6 median fluorescent intensity was calculated on a per-cell basis. Results are representative of two biological replicates.

Plasmids. pCDNA3.1 murine full length myc-MAGI1, myc-MAGI1 deletion mutants and individual myc-MAGI1 domain constructs were generous gifts from Manuela Baccarini (Zmajkovicova, et al., Mol Cell 50, 43-55 (2013).). Human MAGI1c-Y373F-GFP mutant were generated from pCMV6-AC-GFP-MAGI1c WT (Origene, #RG212712) by site directed mutagenesis using the following primers: CATAGTAGATACCAAAGACAGGGTCTTCAATCTTTTCCCAAC (SEQ ID NO: 5), GTTGGGAAAAGATTGAAGACCCTGTCTTTGGTATCTACTATG (SEQ ID NO: 6). pCMV SRC WT was a gift from Jon Cooper. pRK7-HA-S6K1-WT (Addgene #8984), pRK7-HA-S6K1-E389-deltaCT (Addgene #8993) were gifts from J. Cooper and J. Blenis.

CRISPR guide RNA sequences. Guide RNA sequence used to generate human MAGI1 KO in Cas9 expressing stable RBE cells: G*A*A*GGGUUUCGUGUAAAAAA (SEQ ID NO: 7), A*U*C*AAGAGCUUGGUCCUAGA (SEQ ID NO: 8), U*C*G*UGGCUUUGGCUUCACGG (MAGI1) (SEQ ID NO: 9); G*C*A*CUACCAGAGCUAACUCA (non-targeting control) (SEQ ID NO: 10). * indicates 2′-O-methyl analogs and 3′-phosphorothioate internucleotide linkages. All sequences with added Synthego modified EZ scaffold at 3′(Synthego) where *indicates 2′O-methyl analogs and 3′-phosphorothioate internucleotide linkages. All sequences with added Synthego modified EZ scaffold at 3′(Synthego).

Immunohistochemistry and Image Analysis. Samples from patients with ICC. Surgical resected tumor samples from ICC patients were obtained from Dr. Raymond Yeung following UW Medicine (IRB protocol #00001852). Tissue samples were fixed for 7 days in 10% buffered formalin phosphate, embedded in paraffin and sectioned (5 μm thickness) by the FHCC Experimental Histopathology Core. Immunohistochemistry was performed as previously described (Fitamant, et al., Cell Rep 10, 1692-1707 (2015)). Briefly, sections were hydrated followed by antigen retrieval using sodium citrate buffer. Sections were stained using anti-Phospho-S6 Ribosomal Protein (Ser235/236) primary antibody (Cell Signaling #4858) at 1:50 dilution. Biotinylated secondary antibody was used at 1:200. Sections were then stained with Hematoxylin (Thermo Scientific #6765007), dehydrated, and mounted. Stained slides were visualized using the Zeiss Observer.Z1 microscope at 20× magnification. Representative images were captured using uniform brightness and contrast between samples.

PDX tissue samples. Sample preparation and immunohistochemistry experiments were performed as previously described (G. C. McAlister, supra). Briefly, tumors were fixed immediately after excision in a 4% buffered formalin solution for a maximum of 24 hours at RT before being dehydrated and embedded in paraffin. Fixed tissue samples embedded in paraffin were sectioned to a 3-μm thickness, and slides were heated in the instrument at 75° C. for 8 min and deparaffinized with EZ prep solution (Ventana Medical System, catalog no. 950-102 2 L). Antigen retrieval was performed at 95° C. for 64 min using the cell conditioning 1 buffer (Ventana Medical System, catalog no. 950-124 2 L). Subsequent incubation of 8 min with CM inhibitor (ChromoMap DAP kit) was used for peroxidase blockade. For primary antibodies anti-Ki67 (1:250 dilution; Roche, #05278384001; RRID:AB_2631262) and anti-cleaved caspase-3 (Asp175; 1:100 dilution; Cell Signaling Technology, #9661; RRID:AB_2341188), slides were first incubated at 37° C. for 24 or 60 min, respectively, and for a further 8 min with UltraMap anti-rabbit antibody (horseradish peroxidase; Roche, #05269717001; PRID:AB 2924783). As a detection system, a CM ChromoMap DAB kit (Roche, #760-159) was used according to the manufacturer's instructions, followed by counterstaining with hematoxylin II (Ventana Medical System, #760-2021) for 8 to 12 min and bluing reagent (Ventana Medical System, #760-2037) for 4 min, dehydration, and mounting processes. Slides were scanned in the NanoZoomer 2.0-HT slide scanner (Hamamatsu Photonics) and visualized in the NDP.view2 software (Hamamatsu Photonics) or QuPath.

Patient-derived xenografts (PDX) studies. All mouse procedures were conducted in accordance with the Animal Research Reporting of In Vivo Experiments (ARRIVE) guidelines.

PDX ICC195 (IDH WT). All experiments at Fred Hutchinson Cancer Center were conducted under protocol PROT0202000037 and approved by the Institutional Animal Care and Use Committee. Human PDX ICC195 was developed by implanting a fresh resection specimen from a patient with an IDH WT ICC tumor (IRB-approved protocol #00001852). Tumor fragments with the size of 1-2-mm³ were rinsed in RPMI, suspended in Matrigel (Corning), and implanted subcutaneously into the right flanks of 6- to 8-week-old female NSG (NOD scid gamma) mice. When tumors reached 100-200 mm³, mice were randomized into 4 groups for treatment with vehicle control, dasatinib 30 mg/kg, M2698 10 mg/kg, dasatinib 30 mg/kg+M2698 10 mg/kg combo daily by oral gavage for 28 days. All drugs were dissolved in 100 mM Citrate buffer (pH 3). Tumor growth and body weight were monitored 2 times a week. PDX tumors were harvested at the end of treatment.

PDX62 (IDH1 mutant, R132C). The animal procedures conducted at the Vall d'Hebron Institute of Oncology (VHIO) were approved by the Ethical Committee for the Use of Experimental Animals in accordance with the regulations of the Government of Catalonia. PDX62 was generated by subcutaneous implantation of a metastatic liver biopsy from a patient with IDH1 mutant (mIDH1) (R132C) ICC tumor (Serra-Camprubi, et al., Clin Cancer Res 29, 432-445 (2023).). PDX62 tumor pieces (3-4 mm) were subcutaneously implanted into the right flanks of 6-to 8-week-old female NOD.CB-17-Prkdc scid/Rj mice (Janvier Labs, RRID:MGI:3760616). Animals were housed in air-filtered flow cages with a 12:12 light/dark cycle, and food and water were provided ad libitum. Upon xenograft growth (150-200 mm³), PDX62-bearing mice were randomized into 4 groups and treated as PDX ICC Tumor growth was measured twice per week with a caliper; investigators were blinded to treatment effect. Tumor volumes were calculated using the formula: V=(length×width2)/2. Mice weights were recorded 2 times per week. Mice were euthanized by CO2 inhalation when tumors reached 1-1.5 cm³ or severe weight loss occurred, according to institutional guidelines.

Statistical analyses. Statistical significance was determined by specific tests and is presented as means±standard error of the mean (SEM0 as indicated in the figure legends. Statistical analyses were performed using GraphPad Prism. Student's two-tailed t test was used when comparing data from two groups, and one-way or two-way analysis of variance (ANOVA) was used when comparing more than two groups to determine significance, which was set at a P value of <0.05.

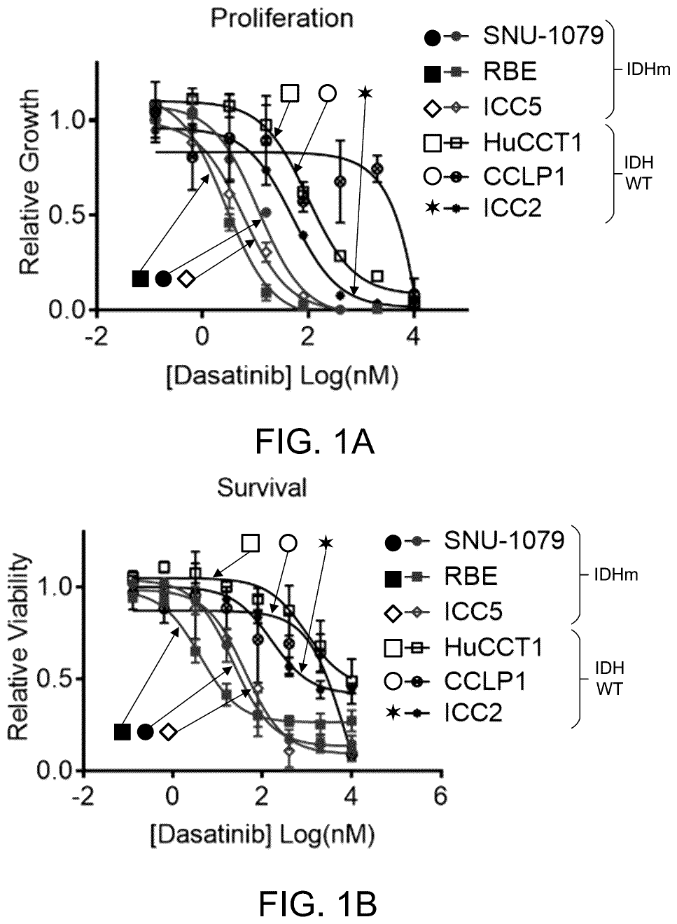

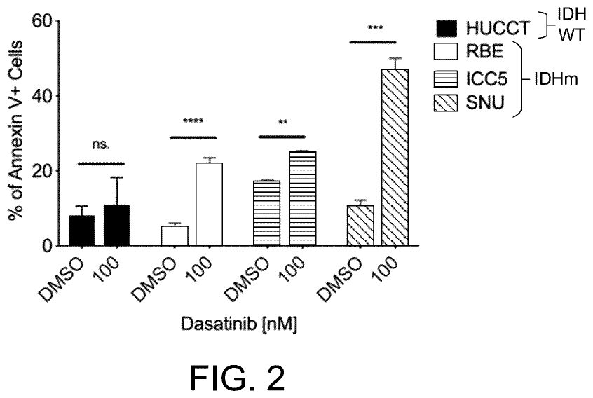

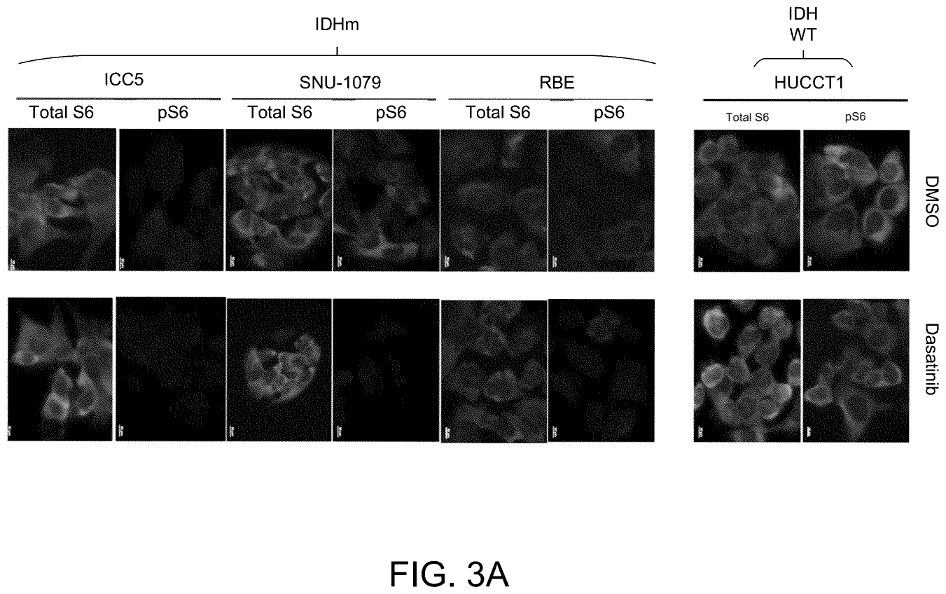

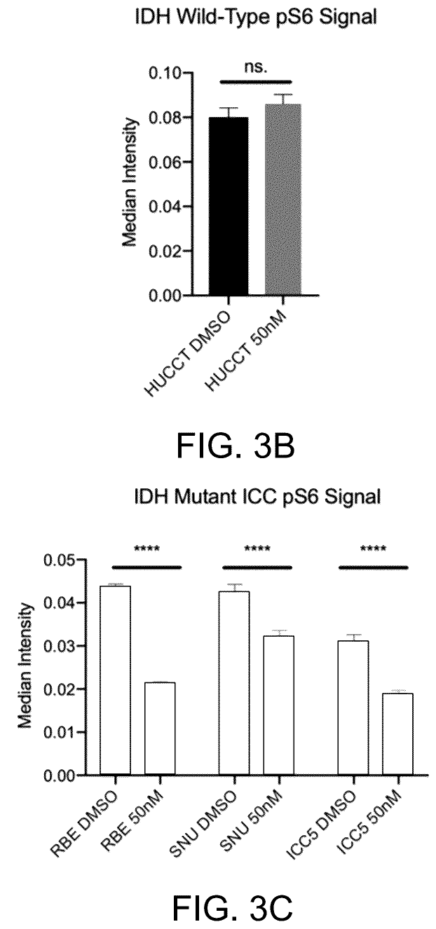

Results. Dasatinib inhibits pS6K, reduces cell size and de novo protein synthesis independent of AKT/mTORC1 in IDHm ICC. IDHm ICC cells are uniquely sensitive to dasatinib (FIGS. 1A, 1B, 2, Table 2). The change in the activity of canonical SRC downstream survival pathways in response to dasatinib was examined in three IDH wild-type (WT) (HuCCT1, CCLP1, and ICC2) and three IDHm (SNU-1079, RBE, and ICC5) cell lines. At 6 hours of dasatinib treatment, while ERK and STAT3 were unaffected, phosphorylation of S6K and its downstream target S6 was completely ablated in all three IDHm lines tested. By contrast, dasatinib had no effect on any of the pathways evaluated in three IDH WT ICC lines in this experimental example. The inhibition was confirmed by immunofluorescence staining of pS6 and total S6 (FIG. 3A-C) Intriguingly, no effect was observed on other upstream (AKT, mTOR) components of the mTOR pathway (FIG. 4A). Moreover, dasatinib treatment did not affect p90RSK and S6K2 phosphorylation (FIG. 4B).

TABLE 2
Proliferation IC₅₀ values from FIG. 1A and survival IC₅₀ values
from FIGS. 1A and 1B.

| Cell Line | Proliferation IC₅₀ | Survival IC₅₀ |
|---|---|---|
| SNU-1079 | 11.40 nM | 22.65 nM |
| RBE | 2.75 nM | 4.10 nM |
| 1CC5 | 5.93 nM | 41.69 nM |
| HuCCT1 | 97.92 nM | 976.7 nM |
| CCLP1 | N.D. | 8043 nM |
| ICC2 | 50.64 nM | 162.3 nM |

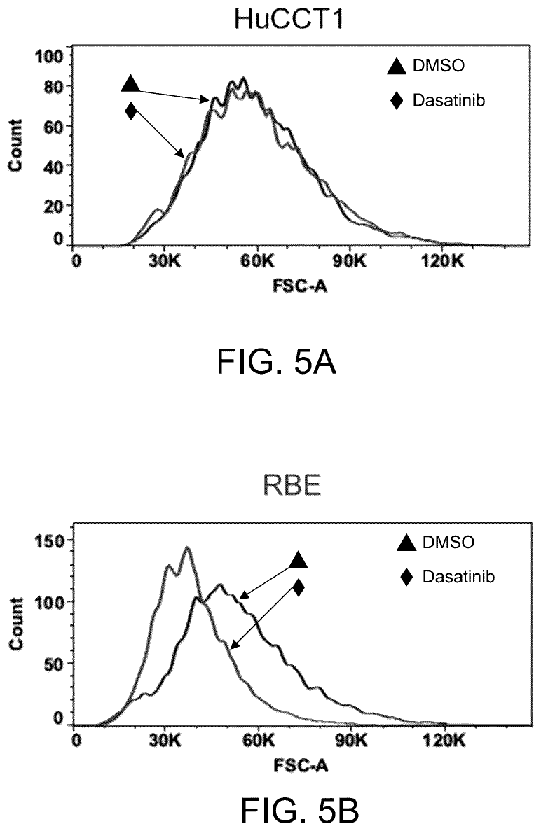

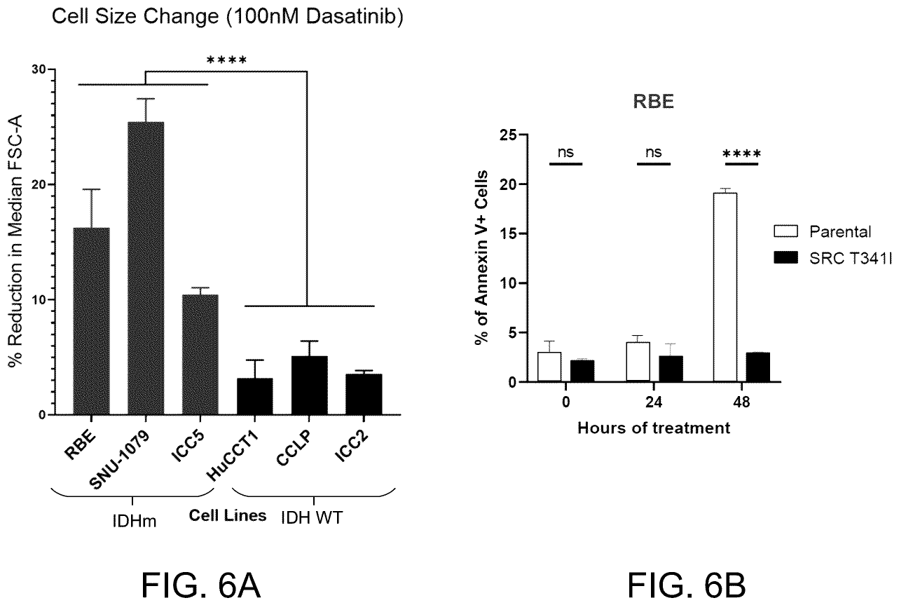

Dasatinib reduced cell size of all three IDHm ICC lines tested as measured by flow cytometry in this experimental example, while having minimal impact on IDH WT cell lines (FIGS. 5A 5B, 6A). The reduction in cell size was observed within 24 h of dasatinib treatment and well before increased apoptosis was detected (FIG. 6B).

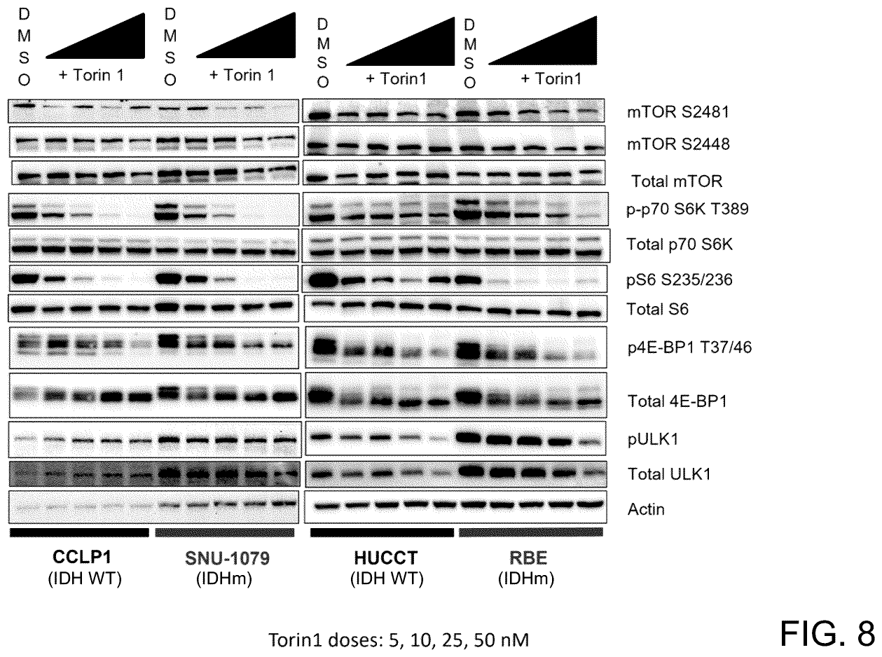

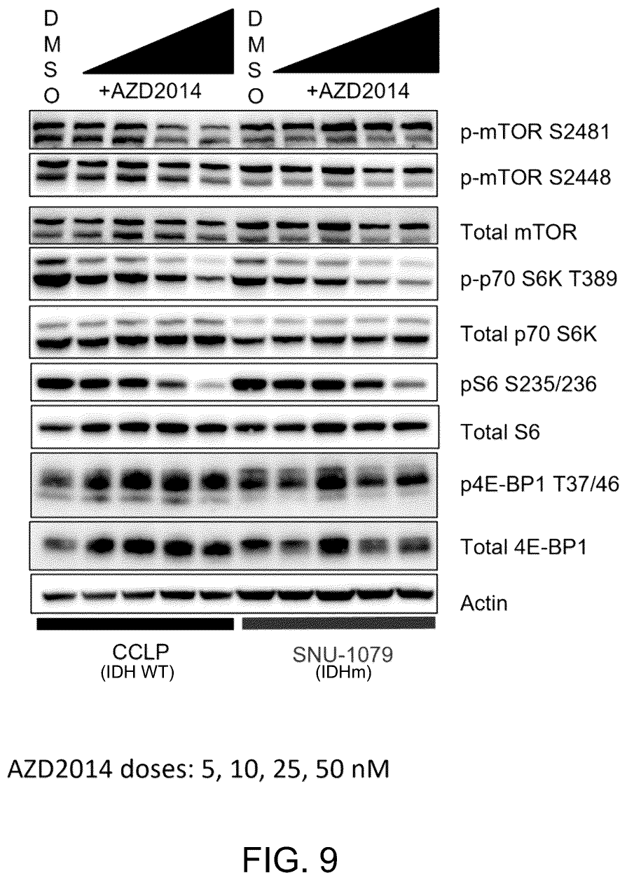

Given that cell size is intimately linked to protein synthesis, de novo protein synthesis of IDHm ICC line upon treatment with dasatinib was measured, using puromycin incorporation as a surrogate. Treating IDHm ICC lines with concentrations of dasatinib as low as 50 nM for just 6 h resulted in a dramatic reduction in global translation, while rates of translation remained robust in IDH WT ICC lines treated with doses as high as 500 nM (FIG. 7). To confirm if the mTOR pathway is intact in IDH WT cells, both IDH WT and mutant cells were challenged with dual mTORC1 and mTORC2 inhibitors Torin1 and AZD2014. Treatment of both mTOR inhibitors result in suppression of pS6K and pS6 at comparable doses (FIGS. 8, 9). Taken together, these results show that dasatinib-induced cell death is associated specifically with the inhibition of S6K/S6 signaling, rapid suppression of protein synthesis, and apoptosis in IDHm ICC cell lines but not their WT counterparts.

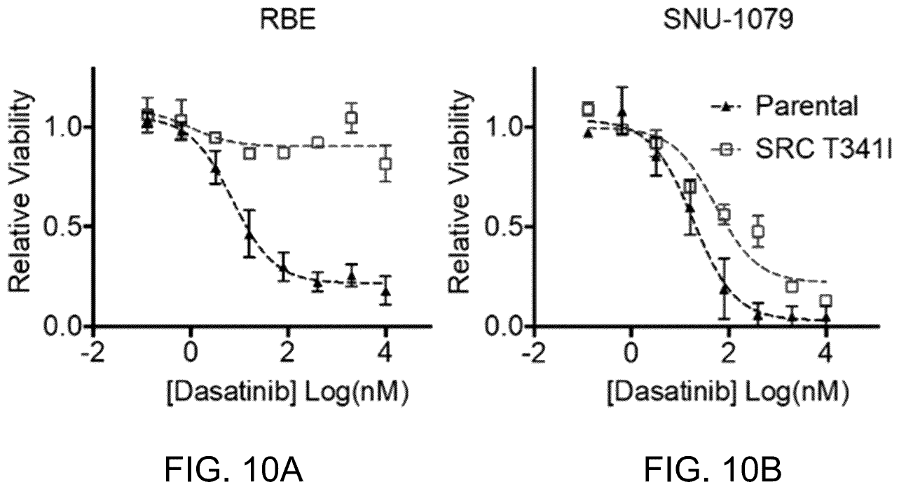

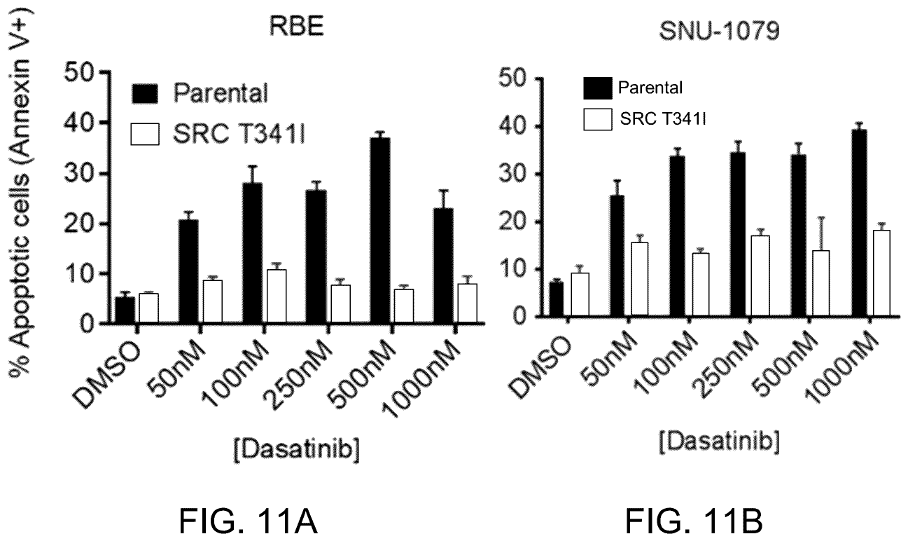

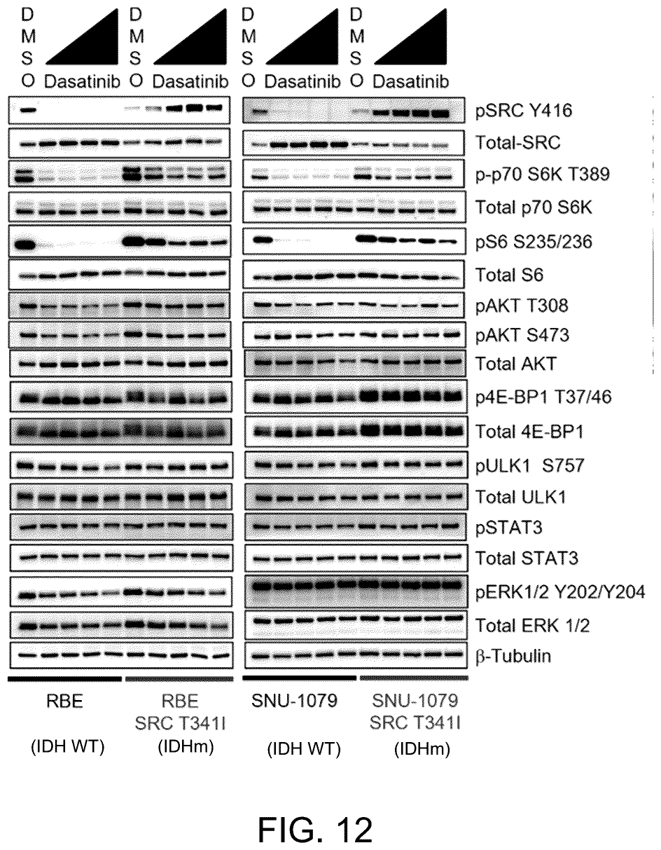

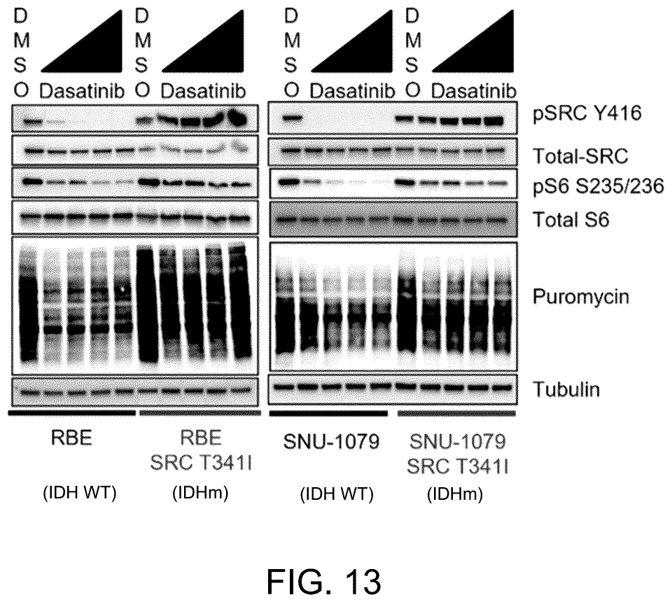

Inhibition of SRC is both necessary and sufficient for killing IDHm ICC through inhibition of S6K/S6 signaling. Two endogenous SRC T341I ‘gatekeeper’ mutant IDHm ICC cell lines were previously generated (Saha, et al., Cancer Discov 6, 727-739 (2016)). Both SRC T341I mutants were highly resistant to growth inhibition and apoptosis by dasatinib compared to their parental counterparts at increasing dasatinib concentrations (FIGS. 10A, 10B, 11A, 11B). The resistant phenotype in the SRC gatekeeper lines in both SNU-1079 and RBE was associated with rescue of the pSRC Y416 mark as well as pS6K and pS6 levels (FIG. 12). None of the other survival signaling pathways, such as pAKT, pSTAT3, pERK and other mTOR downstream targets such as p4E-BP1 and pULK were affected (FIG. 12). Furthermore, dasatinib also failed to inhibit de novo protein synthesis as indicated by sustained puromycin uptake in either SNU-1079 or RBE SRC T341I lines, even at concentrations as high as 500 nM (FIG. 13). These results show that SRC is a necessary target to bring about the cytotoxic and translation inhibitory effects of dasatinib.

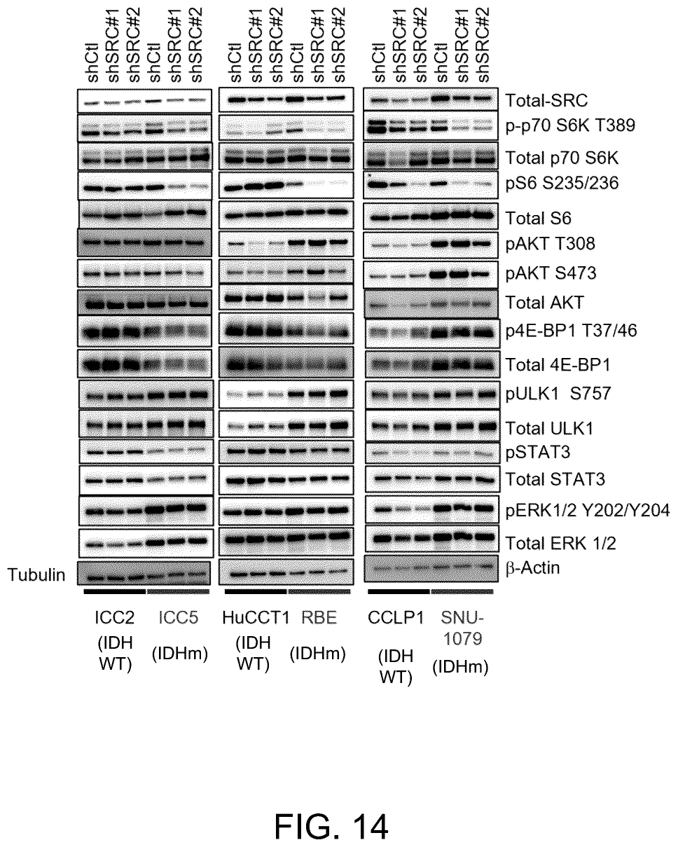

To further investigate whether inhibiting SRC alone was sufficient in suppressing S6K signaling, SRC was knocked down (KD) in three IDHm and three IDH WT ICC lines using two independent shRNA hairpins. SRC KD resulted in reduced pS6K and pS6 in all three IDHm ICC lines, while having no effect on any of the IDH WT lines. In agreement with dasatinib treatment, SRC knockdown did not change other phosphorylation marks including pERK, pSTAT3, pAKT, p4E-BP1 and pULK1 (FIG. 14). Taken together, these results demonstrate that SRC promotes cell survival and regulates both pS6K/pS6 signaling axis and protein translation in IDHm but not IDH WT ICC.

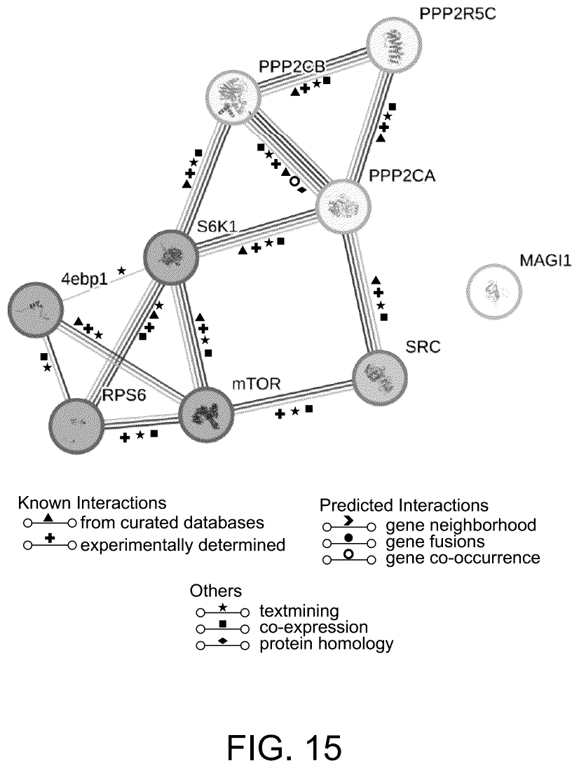

MAGI1 is a novel substrate of SRC and modulates downstream S6K signaling. A Search Tool for the Retrieval of Interacting Genes/Proteins (STRING) analysis was performed that did not identify any known interaction between SRC and S6K (FIG. 15A, Table 3). Based on the fact that SRC is a tyrosine kinase and both S6K and S6 are activated by phosphorylation of serine/threonine residues, it appears that one or more intermediate molecules are involved. Furthermore, no evidence that other upstream or downstream components of the mTOR pathway were affected by SRC inhibition was observed.

TABLE 3
The combined score of individual protein node interaction,
range from 0-1, with 1 being the highest interaction.

| Node 1 | Node 2 | Combined Score |
|---|---|---|
| 4ebp1 | S6K1 | 0.972 |
| 4ebp1 | RPS6 | 0.931 |
| 4ebp1 | mTOR | 0.999 |
| PPP2CA | PPP2CB | 0.986 |
| PPP2CA | S6K1 | 0.979 |
| PPP2CA | SRC | 0.822 |
| PPP2CA | PPP2R5C | 0.999 |
| PPP2CB | S6K1 | 0.92 |
| PPP2CB | PPP2CA | 0.986 |
| PPP2CB | PPP2R5C | 0.995 |
| PPP2R5C | PPP2CB | 0.995 |
| PPP2R5C | PPP2CA | 0.999 |
| RPS6 | S6K1 | 0.999 |
| RPS6 | 4ebp1 | 0.931 |
| RPS6 | mTOR | 0.961 |
| S6K1 | PPP2CB | 0.92 |
| S6K1 | 4ebp1 | 0.972 |
| S6K1 | PPP2CA | 0.979 |
| S6K1 | RPS6 | 0.999 |
| S6K1 | mTOR | 0.999 |
| SRC | mTOR | 0.793 |
| SRC | PPP2CA | 0.822 |
| mTOR | S6K1 | 0.999 |
| mTOR | 4ebp1 | 0.999 |
| mTOR | SRC | 0.793 |
| mTOR | RPS6 | 0.961 |

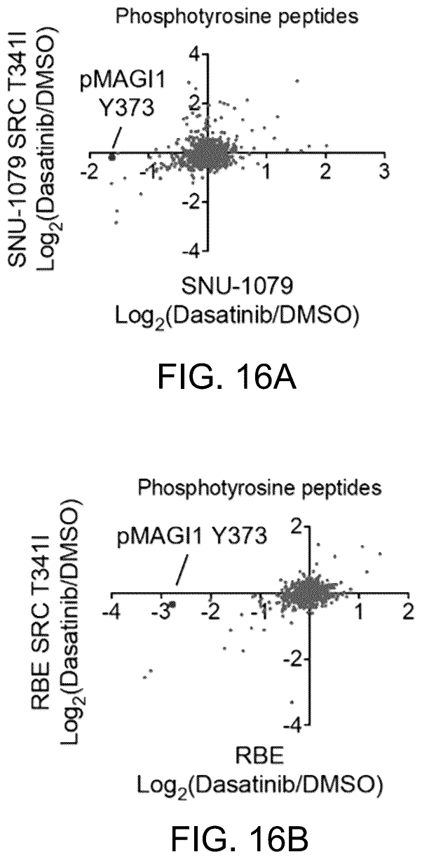

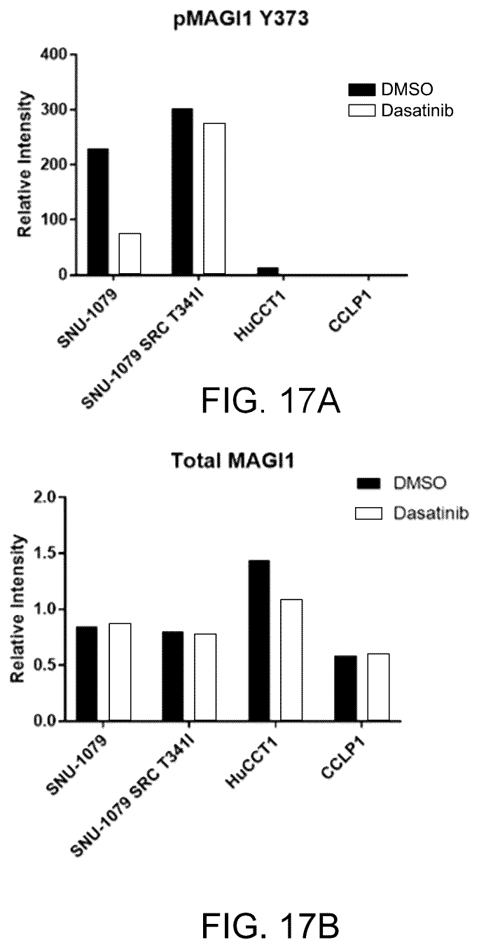

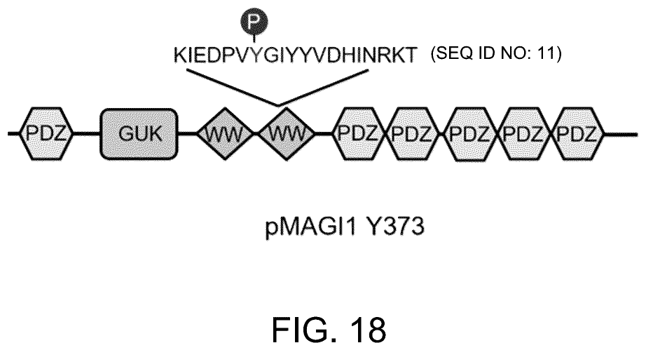

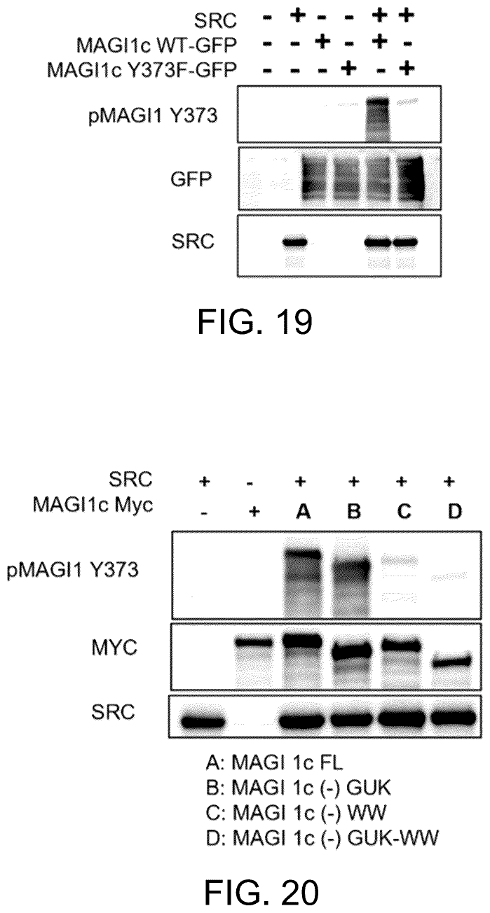

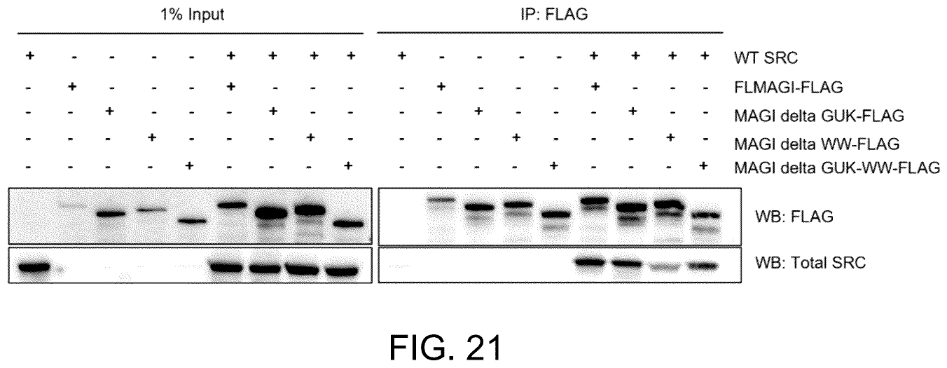

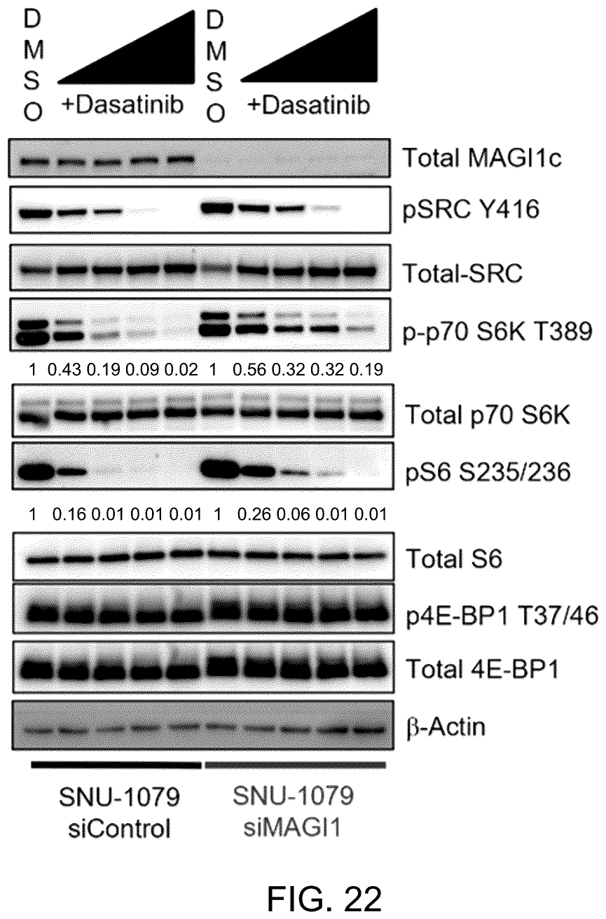

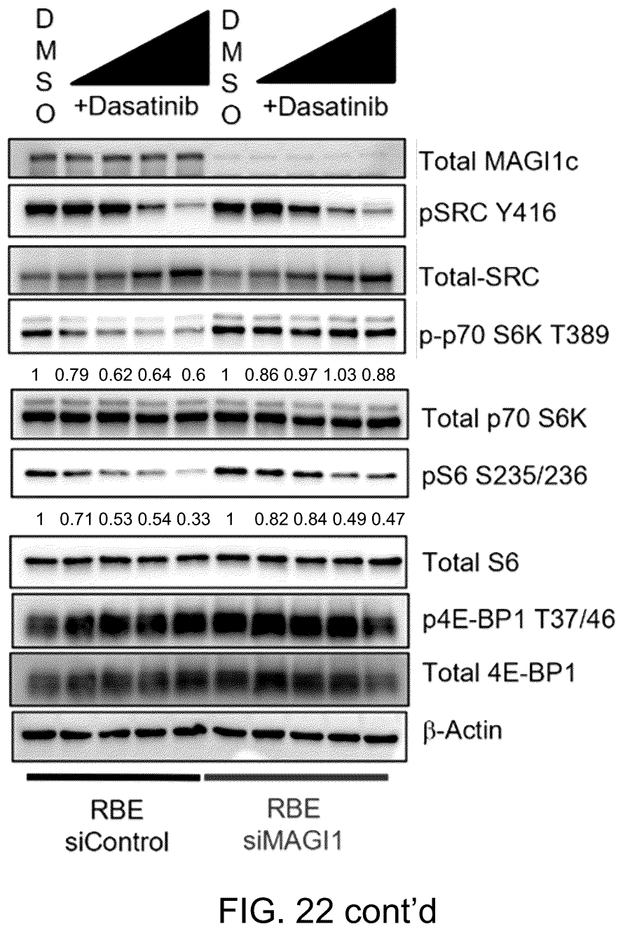

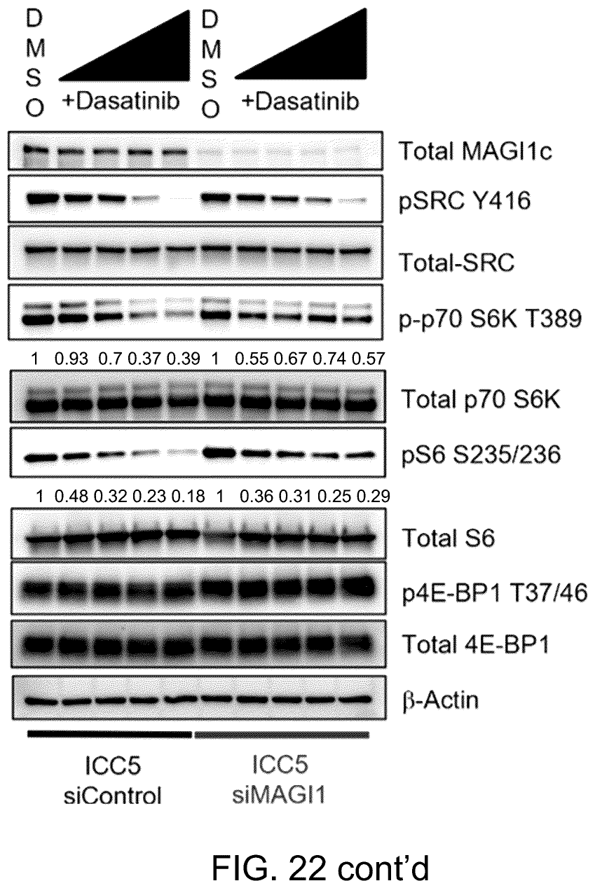

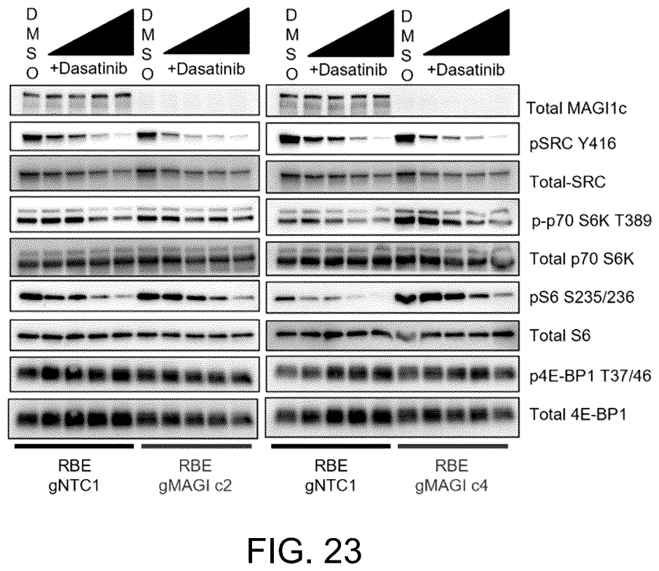

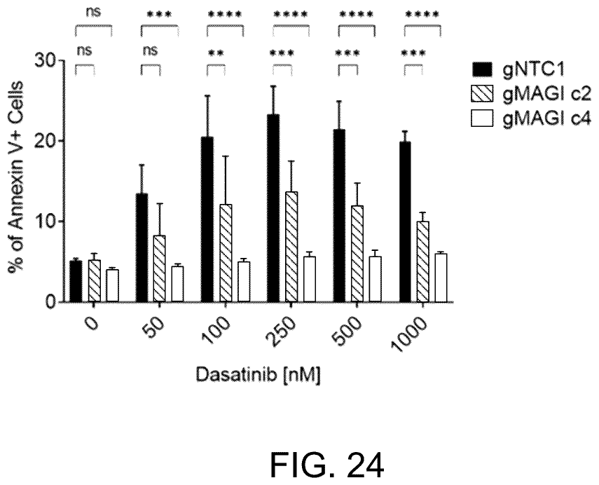

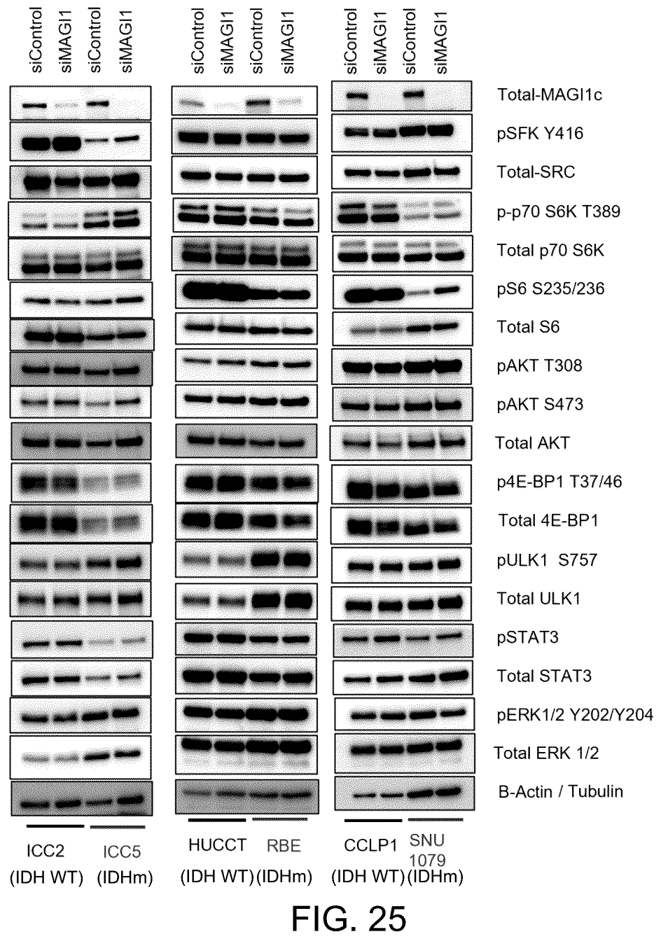

Two IDHm ICC SRC WT and T341I isogenic cell line pairs were treated with dasatinib at 20 nM for 1 hour. Phosphopeptides were extracted from the tryptic digests of the protein lysates, and were then subjected to mass spectrometry-based multiplexed quantitative phosphoproteomics in order to characterize dynamic changes in the phosphoproteome (J. Lyons, supra; J. Kreuzer, supra). Top ‘hits’ would be represented by phosphotyrosine peptides that were significantly reduced in abundance following dasatinib treatment in parental SRC WT lines, but not affected by dasatinib treatment in SRC T341I isogenic derivatives. Surprisingly, the same phosphopeptide, phosphorylated Membrane Associated Guanylate Kinase, WW And PDZ Domain Containing 1 (MAGI1) Y373, represented the top ‘hit’ in both SRC WT/T341I pairs tested (FIGS. 16A, 16B). By contrast, pMAGI1 Y373 was barely detectable in both IDH WT ICC cell lines tested (FIGS. 17A, 17B). MAGI1 is a large scaffolding protein with six PDZ domains, two WW domains and a kinase dead guanylate kinase domain. It is thought to localize to tight junctions and function as a tumor suppressor (Zaric, et al., Oncogene 31, 48-59 (2012); Laura, et al., Exp Cell Res 275, 155-170 (2002)). One study identified a potential role for MAGI1 in the PTEN/PI3K/Akt pathway (Zmajkovicova, et al., Mol Cell 50, 43-55 (2013)). pMAGI Y373 can be found in the second WW domain (FIG. 18) and has been previously described to be a potential substrate for the Ptprz phosphatase (Fujikawa, et al., J Biol Chem 286, 37137-37146 (2011)). To explore whether MAGI1 may serve as a novel SRC substrate, rabbit antisera against pMAGI1 Y373 was raised, and SRC and either WT MAGI1 or a MAGI1 Y373F mutant (which cannot be phosphorylated) were co-expressed in 293T cells. Indeed, co-expression of SRC and WT MAGI1 but not Y373F mutant resulted in strong phosphorylation of MAGI1 at Y373 in this example (FIG. 19). Co-expression of SRC with WT MAGI1 full length and truncation mutants lacking either GUK, WW or GUK-WW domains further demonstrated that Y373 phosphorylation was abolished when WW domain was absent but not GUK (FIG. 20). The interaction between SRC and MAGI1 was confirmed by co-immunoprecipitation in 293T cells where SRC strongly binds MAGI1 full length and GUK deletion truncation mutant. The WW and GUK-WW truncation mutants show reduced binding with SRC, with WW truncation mutant having the lowest affinity in this example (FIG. 21). It was next determined whether MAGI1 may play a functional role in the S6K/S6 pathway. First, three different IDHm ICC cell lines were transfected with either control siRNA or pooled siRNA against MAGI1 and treated with increasing doses of dasatinib. KD of MAGI1 partially rescued pS6K/pS6 in all three lines tested whereas p4E-BP1 remained unchanged in this example (FIG. 22). To further corroborate these findings, CRISPR/Cas9 editing was used to generate two MAGI1 knockout (KO) RBE clones. Similar to the KD cells, dasatinib treatment in both KO clones resulted in partial rescue of pS6K/pS6 levels (FIG. 23) as well as resistance to dasatinib-induced apoptosis (FIG. 24). Additionally, KD as well as KO of MAGI1 led to a modest increase in pS6 even in DMSO treated cells (FIGS. 22, 23). In this experimental example, MAGI1 acts as a negative regulator of the S6K/S6 pathway. To examine if MAGI1 regulated other components of the mTOR pathway, MAGI1 was knocked down in multiple human ICC lines and it was found that MAG1 did not affect mTOR signaling in either IDH WT or IDHm cells (FIG. 25). Based on these results, SRC-mediated phosphorylation of MAGI1 acts as an inhibitory modification in this experimental example, preventing MAGI1 from suppressing the S6K/S6 pathway. In dasatinib-treated cells, SRC can no longer phosphorylate MAGI1, allowing it to inhibit phosphorylation of S6K.

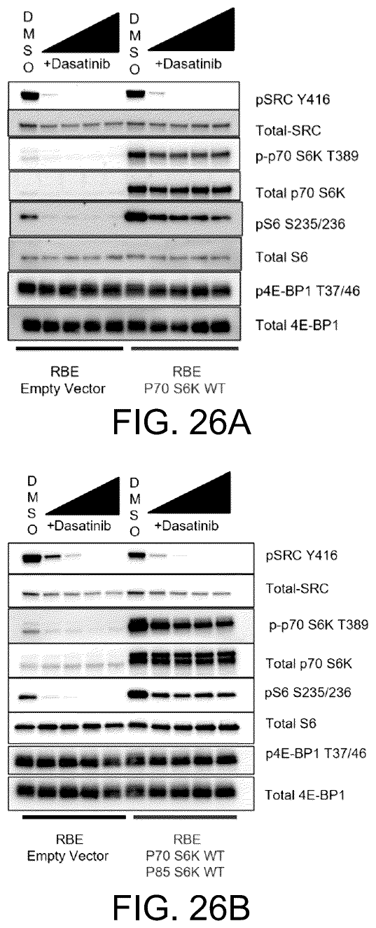

Dasatinib inhibits S6K/S6 signaling through activation of PP2A. As a scaffolding molecule, without intrinsic kinase activity, it is unlikely that MAGI1 directly regulates phosphorylation of S6K. T389 of S6K is a direct substrate of mTOR when complexed with Raptor (mTORC1); however, no evidence that either upstream or downstream components of the mTORC1 pathway were affected by dasatinib treatment was observed. In addition, over-expressing constitutively active S6K failed to rescue S6K/S6 signaling (FIGS. 26A, 26B). Based on these results, SRC/MAGI1 may regulate S6K in an mTOR-independent manner. It was next studied whether a phosphatase may instead be recruited to mediate SRC/MAGI1 regulation of S6K. Of the known human phosphatases, PP2A has been shown to directly bind to S6K and dephosphorylate T389 without affecting phosphorylation of AKT or 4E-BP (Hahn, et al., Cell Metab 11, 438-444 (2010)).

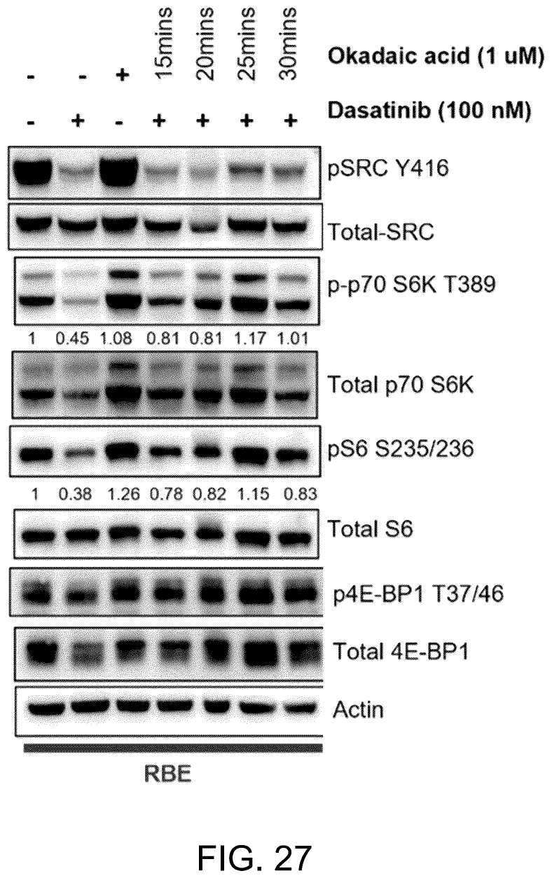

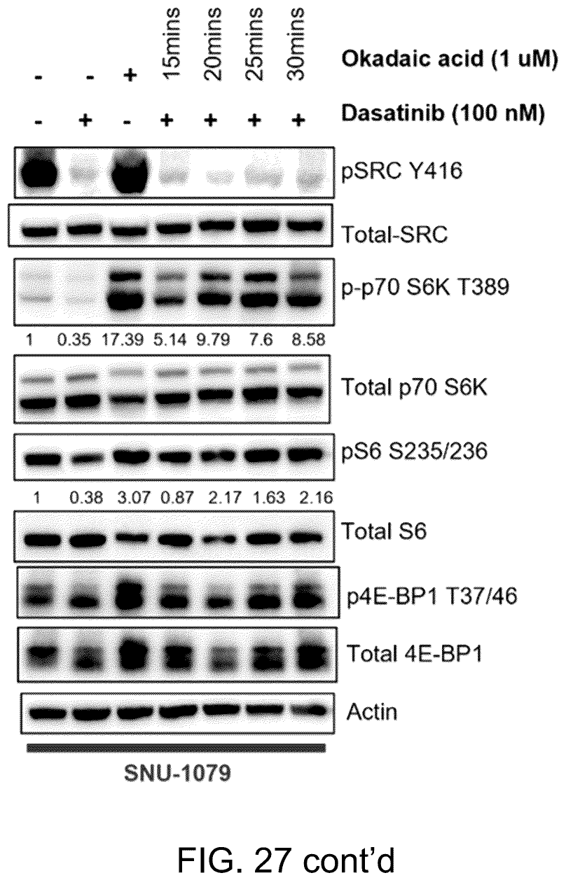

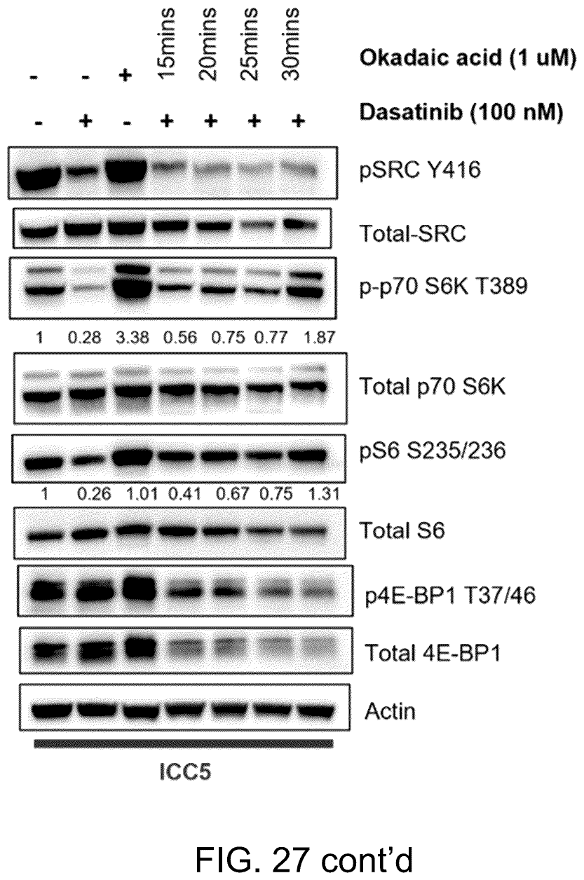

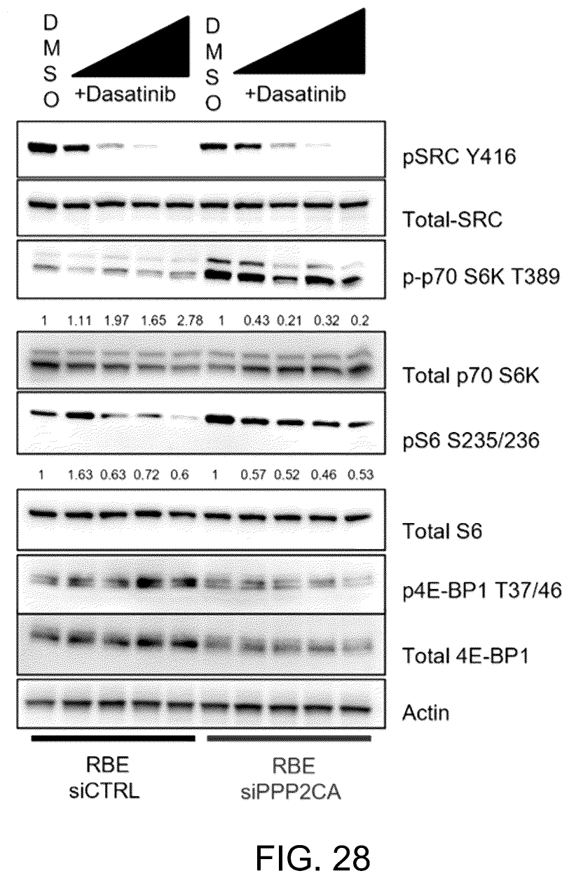

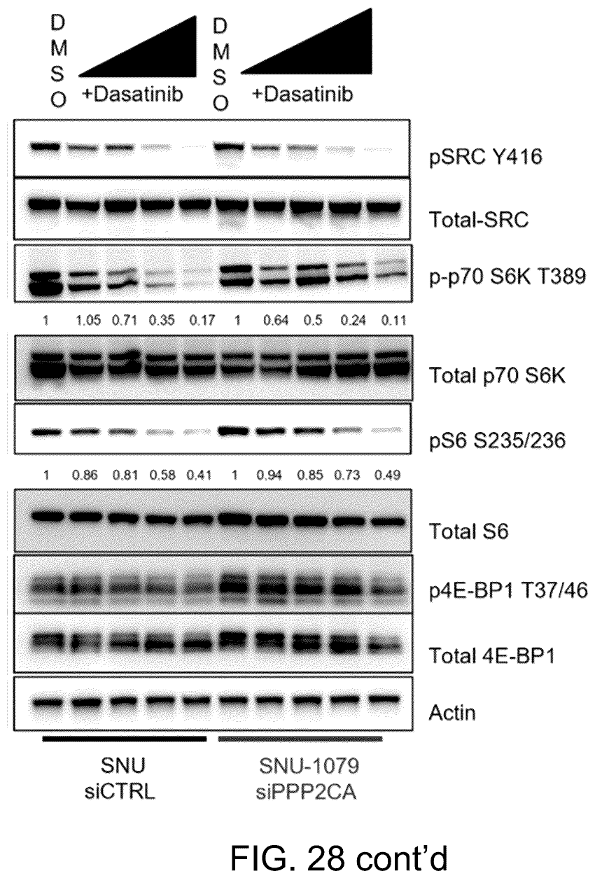

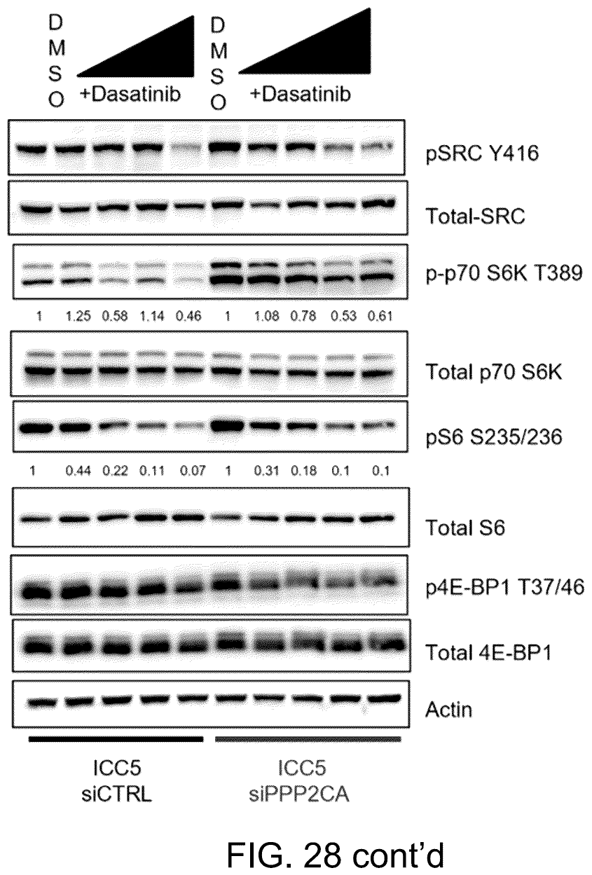

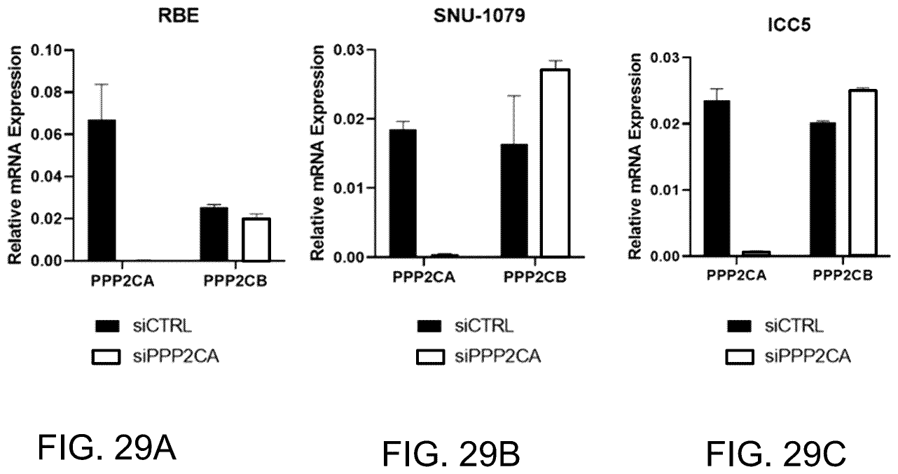

The PP2A holoenzyme is composed of three subunits, A (structural), B (regulatory) and C (catalytic). The human genome encodes only two distinct PP2A-A subunits and two distinct PP2A-C subunits but there are at least 12 different PP2A-B genes that allow for a variety of regulatory mechanisms and substrate specificities (Sangodkar, et al., FEBS J 283, 1004-1024 (2016)). To determine whether PP2A may dephosphorylate S6K T389 in dasatinib-treated IDHm ICC cells, cells with were co-treated okadaic acid, a potent and specific inhibitor of PP2A (cell-free IC50=0.1 nM) and dasatinib. Okadaic acid was able to reverse dasatinib-mediated inhibition of pS6K and pS6 in a time-dependent manner in all three IDHm lines, with rescue seen as early as 15 mins of treatment (FIG. 27). To further verify that the inhibition of pS6K and pS6 came from PP2A phosphatase activity, the dominant form of PP2A catalytic subunit was knocked down by transfecting pooled siRNA against PPP2CA in three IDHm lines. PPP2CA KD elevated baseline S6K and S6 phosphorylation in this experimental example, as well as partially rescued pS6K and pS6 levels upon dasatinib treatment. Neither SRC nor 4E-BP1 phosphorylation were affected, suggesting a specific phosphatase activity targeted against the S6K/S6 axis (FIG. 28). Knockdown was confirmed by qRTPCR showing that the catalytic A subunit was lost with no effect on the catalytic B subunit (FIGS. 29A-29C).

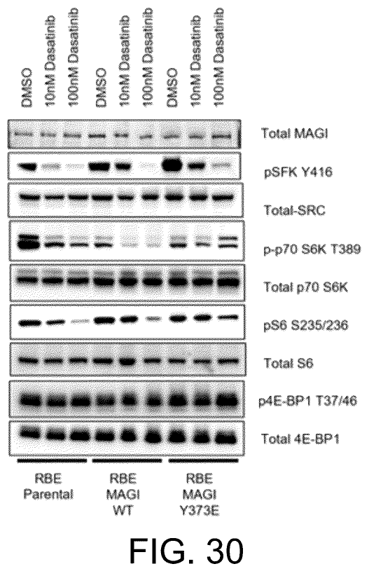

To confirm if phosphorylated MAGI1 is necessary to maintain pS6K, MAGI1 Y373E phospho-mimetic and MAGI1 wild type re-expressing lines were generated in RBE MAGI1 KO cells. When challenged with dasatinib, MAGI1 Y373E phospho-mimetic mutant was able to rescue pS6K and pS6 levels as compared to MAGI1 WT re-expressing and parental RBE lines, while p4E-BP1 remained unchanged (FIG. 30). Taken together, these results show that SRC-mediated S6K/S6 survival signaling is facilitated through binding and direct phosphorylation of MAGI1 at Y373, which then relieves the inhibitory effect of PP2A on S6K/S6 in this Example.

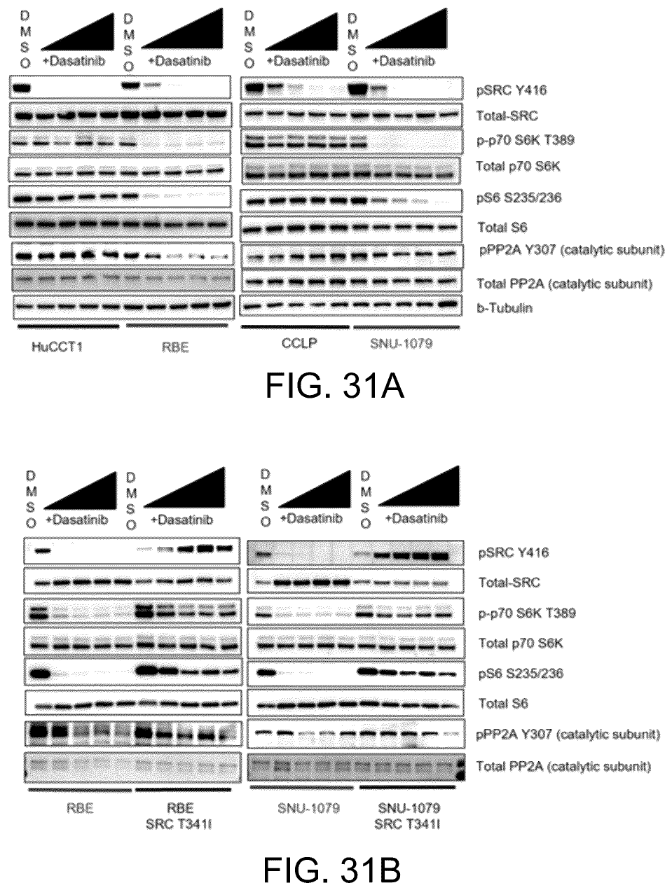

It was next studied if dasatinib treatment either directly or indirectly changed PP2A catalytic activity, which is thought to be modulated by two major post-translational modifications, pY307 (generally repressive) and methyl-L309 (generally activating) (Janssens, et al., Trends Biochem Sci 33, 113-121 (2008)). Phosphorylation of PP2A catalytic subunit (PP2AC) at Y307 decreases its activity by effectively inhibiting the interaction between the catalytic and regulatory subunits of the holoenzyme, which affects proper trafficking to the target (Chen, et al., Science 257, 1261-1264 (1992).; Seshacharyulu, et al., Cancer Lett 335, 9-18 (2013)). This was of particular interest in the study as PP2AC pY307 has been shown to correlate with SRC activity in some contexts (Chen, et al., Science 257, 1261-1264 (1992)), and re-activation of PP2A has become a therapeutic avenue of interest for a number of different cancer types (Farrington, et al., J Biol Chem 295, 757-770 (2020); Tohme, et al., JCI Insight 4, (2019); Shenolikar, J Clin Invest 127, 2048-2050 (2017)). To understand the role of this relationship in IDHm ICC, and to see if re-activation of PP2A may be occurring through inhibition of SRC, the panel of cell lines was treated with dasatinib and probed for pY307 by western blot. IDHm ICC demonstrated a dose-dependent reduction in PP2A pY307 after treatment with dasatinib. This reduction did not occur in IDH WT cell lines (FIG. 31A). Moreover, the SRC gatekeeper IDHm lines partially rescued dasatinib-induced decrease in PP2AC pY307 (FIG. 31B). These findings show that SRC phosphorylates MAGI1 Y373 and thereby inhibits PP2AC through posttranslational modifications.

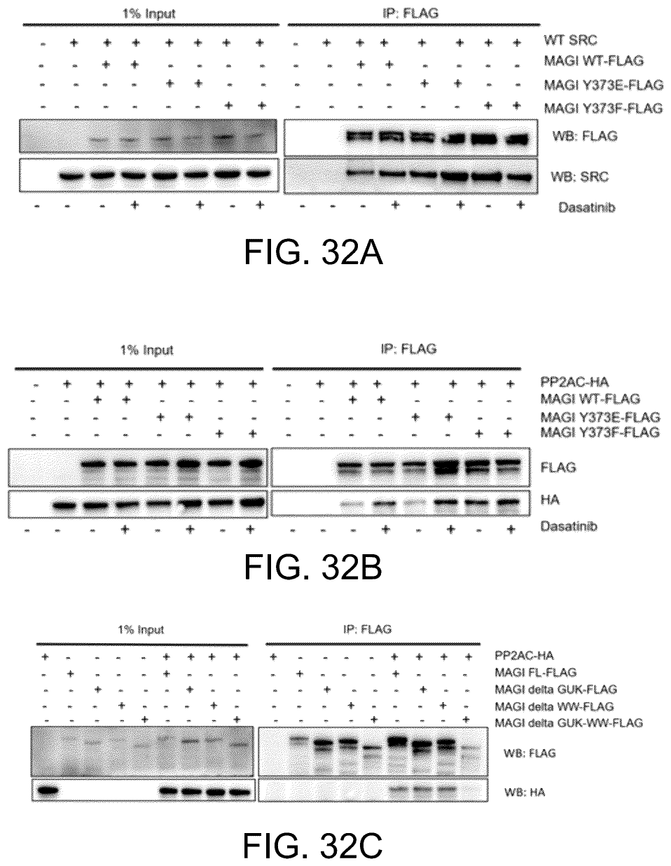

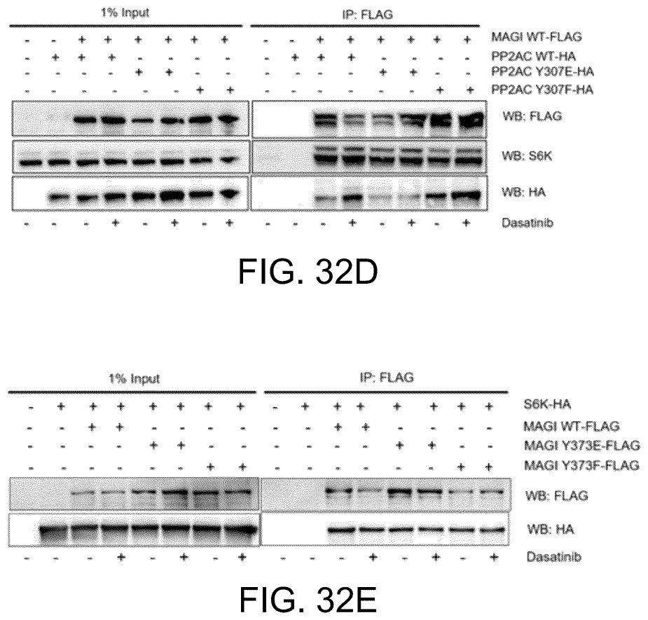

SRC relieves growth suppressive MAGI1-PP2A complex to activate S6K. Given that MAGI1 is a scaffolding protein, it was next determined whether MAG1 could serve to bring together SRC and PP2AC into a signaling complex that could regulate S6K. First, it was examined whether MAGI1 phosphorylation status at Y373 influences SRC binding to MAGI1. SRC was co-immunoprecipitated (co-IP) with either MAGI1 WT, Y373E (phosphomimetic) or MAGI1 Y373F (non-phosphorylatable) mutants, with or without dasatinib treatment. The MAGI Y373F mutant displayed highest binding to SRC. Dasatinib treatment led to increased SRC binding for both WT MAGI and MAGI Y373E mutant (FIG. 32A). This increase was not observed for the MAGI Y373F mutant. Dasatinib belongs to a class of inhibitors that stabilizes the open conformation of SRC. However, SRC inhibition does not seem to affect SRC binding to a non-phosphorylatable target in this experimental example. This data shows that though phosphorylation status of MAGI Y373 influences SRC binding, it is not correlated with SRC activity in this example.

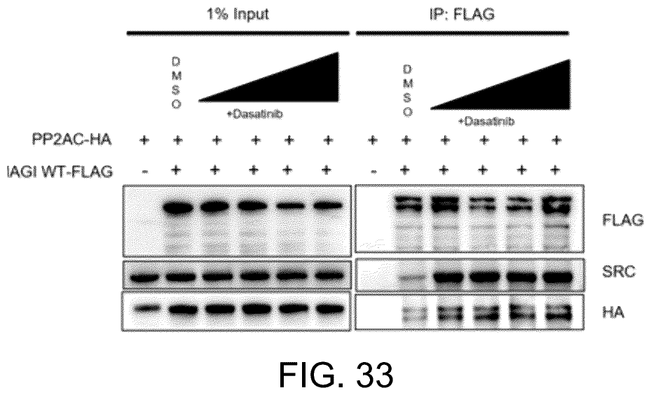

Next, the physical interaction between MAGI1 and PP2AC was examined. Co-IP of PP2AC and MAGI1 in 293T cells demonstrated that PP2AC interacts with MAGI1, and the binding is significantly increased upon dasatinib treatment (FIGS. 32B, 33). In this experimental example, MAGI1 recruits a suppressive signaling complex upon SRC inhibition that facilitates downstream dephosphorylation of S6K. Co-IP of PP2AC with MAGI1 Y373E and Y373F mutants with or without dasatinib treatment was also performed. Similar to SRC, PP2AC appears to bind to MAGI1 Y373F more avidly than Y373E and this binding did not increase with SRC inhibition (FIG. 32B). These results show that SRC-mediated phosphorylation of MAGI1 serves to inhibit PP2AC binding in this example. To determine the MAGI1 domain that binds to PP2AC, WT MAGI1 full length and truncation mutants lacking either GUK, WW or GUK-WW domains were co-expressed with PP2AC. It was found that PP2AC binds less to the truncation mutant lacking both the GUK and WW domains (FIG. 32C). Next, it was determined whether PP2AC phosphorylation status at Y307 and activity influences binding to MAGI1. Co-IP of MAGI1 with either WT, Y307E (phosphomimetic) or Y307F (non-phosphorylatable) PP2AC with or without dasatinib treatment was performed. PP2AC Y307E has weaker whereas Y307F has stronger binding to MAGI1 at baseline in this example. While dasatinib treatment results in increased binding of both WT and Y307F PP2AC to MAGI1, Y307E phosphomimetic mutant does not show any change. This data shows that phosphorylated or inactive PP2AC Y307 cannot bind MAGI1 thus preventing de-phosphorylation of S6K/S6 signaling (FIG. 32D). Endogenous S6K bound to MAGI1 equally well with or without dasatinib when co-expressed with PP2AC WT and Y307 mutants (FIG. 32D). To further confirm S6K binding to MAGI1, co-IP between MAGI1 WT or Y373E/F mutants (flag tagged) with S6K (HA tagged) with or without dasatinib was performed. S6K interacted equally with the WT and MAGI1 phospho-mutants, and the interaction did not change upon dasatinib treatment (FIG. 32E). These results show that it is the PP2A-MAGI1 interaction that acts as a determinant of pS6K/pS6 signaling in this experimental example.

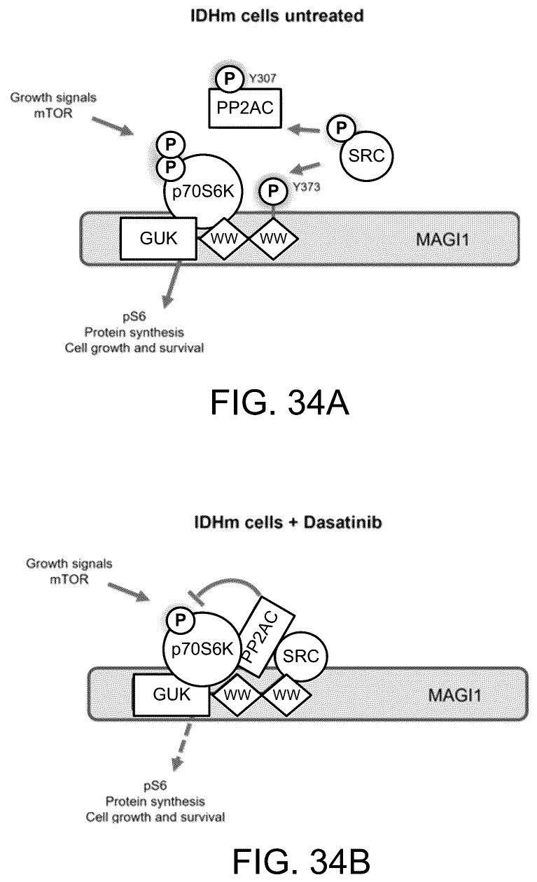

Taken together, these results show that SRC phosphorylation of MAGI1 Y373 and PP2A Y307 prevents the formation of a suppressive MAGI1-mediated signaling complex. This limits access of PP2AC to S6K and leads to S6K/S6 hyperactivation. Inhibition of SRC allows PP2A to bind MAGI1 and de-phosphorylate MAGI1-bound S6K (FIGS. 34A, 34B).

As shown in FIG. 34A, under normal conditions, p70S6K (S6K), PP2AC and SRC physically interact with MAGI1. In IDHm cells at untreated condition, active SRC (in green) phosphorylates MAGI1 at tyrosine 373 at the WW2 domain. When MAGI1 is phosphorylated, PP2AC and SRC bind lesser to MAGI1 whereas S6K binding is unchanged. Active SRC has been shown to phosphorylate PP2AC at Y307 which is an inhibitory modification (in red) that limits its phosphatase activity towards S6K. Phosphorylation status of PP2AC also plays important role in its binding to MAGI1. Phosphorylated PP2AC (inactive, in red) shows weaker affinity to WT MAGI1 than its unphosphorylated form. Lesser PP2AC activity and binding to MAGI1 associates with sustained S6K phosphorylation by mTOR and downstream survival signaling.

As shown in FIG. 34B, when IDHm cells are treated with dasatinib, SRC is inhibited (in red) and resulted in reduced MAGI1 Y373 phosphorylation. Unphosphorylated MAGI1 significantly increases its binding to PP2AC and SRC. Meanwhile, PP2AC Y307 inhibitory phosphorylation is also reduced after SRC inhibition which resulted in more active PP2AC. Active PP2AC (in green) is brought in proximity with S6K by MAGI1 and facilitates S6K dephosphorylation and hence suppression of downstream protein synthesis and survival. Thus, unphosphorylated MAGI1 (or Y373F unphosphorylatable mutant) facilitates SRC-MAGI1-PP2AC complex formation. Also, unphosphorylated PP2AC (active, in green) is associated much stronger to MAGI1 as shown by Y307F unphosphorylatable mutant and dasatinib treatment. Together, SRC inhibition in IDHm cells reduces the phosphorylation of both MAGI1 and PP2AC which result in increased formation of MAGI1-PP2AC complex that negatively regulated mTOR downstream signaling specifically in the S6K/S6 axis.

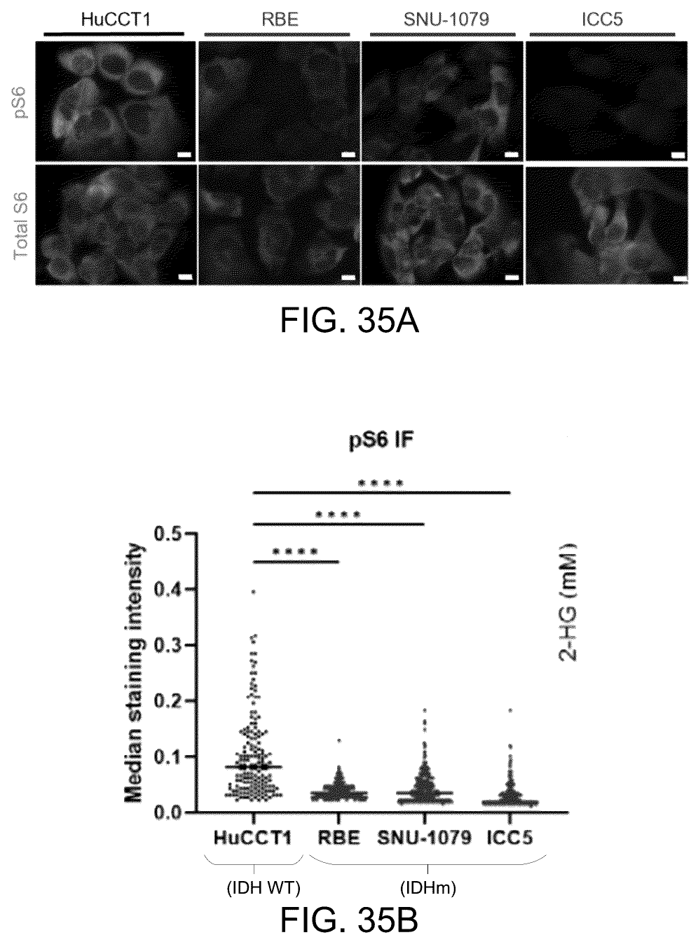

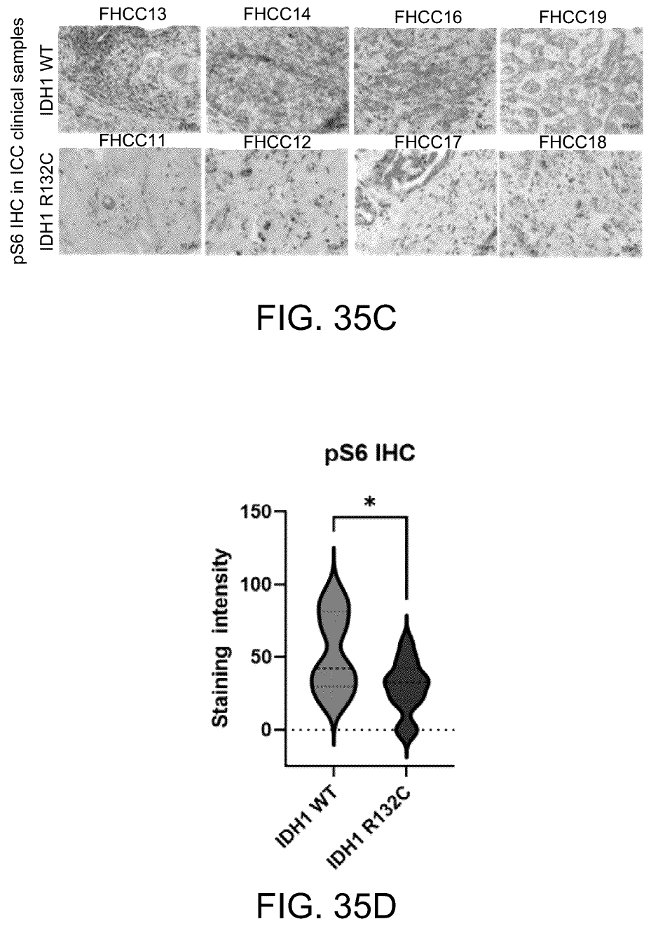

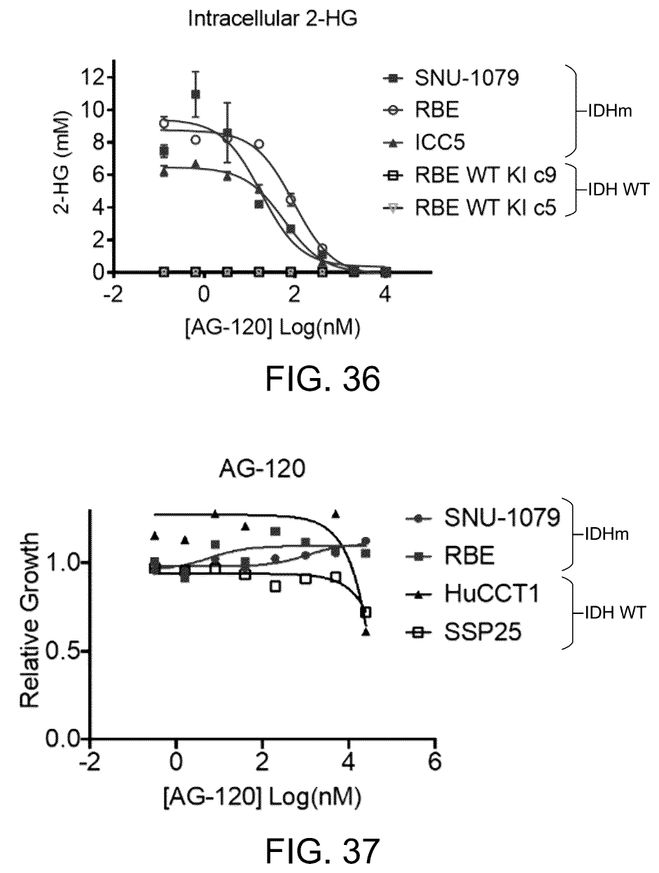

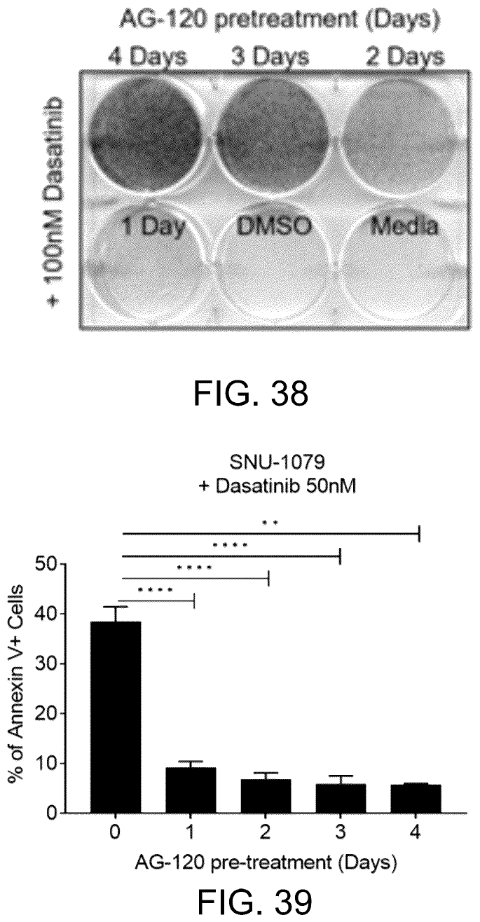

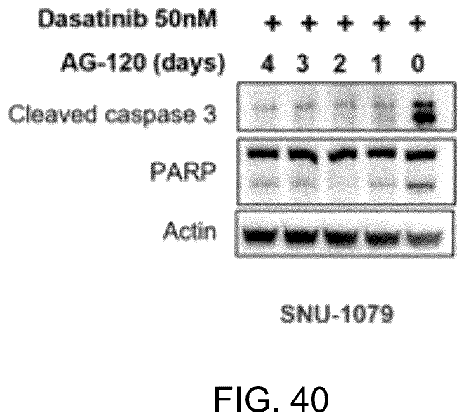

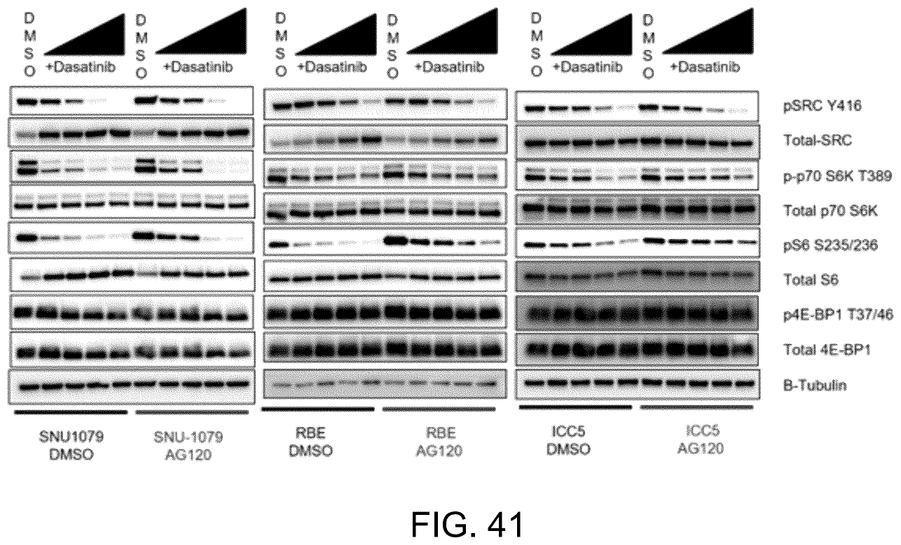

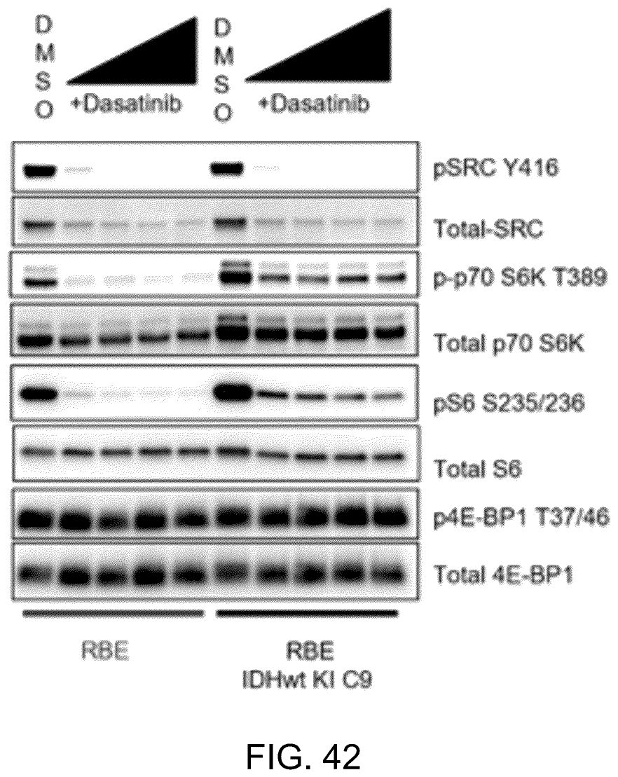

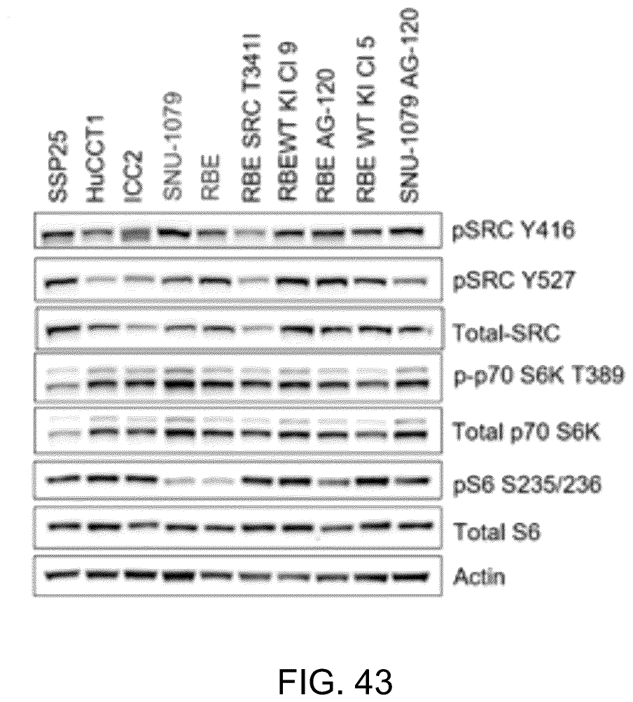

Elevated pS6 levels determine intrinsic and acquired resistance to dasatinib. The results described herein show that IDHm ICC is exquisitely dependent on SRC for cell survival. Intriguingly, IDHm cell lines had much lower levels of pS6 compared to IDH WT (FIGS. 35A, 35B). This finding was recapitulated by immunohistochemistry on samples obtained from patients with ICC (FIGS. 35C, 35D), showing that higher baseline levels of pS6 are what determine resistance. The role of mutant IDH in the response to dasatinib was studied. Mutant IDH1 and the oncometabolite 2-hydroxyglutarate (2HG) it produces was inhibited with a highly potent and specific pharmacological inhibitor, Ivosidenib (AG-120). Treatment with AG-120 in all three human IDHm ICC lines effectively inhibited cellular 2-HG levels with average IC50 of 90 nM, without causing significant toxicity using doses as high as 10 μM in proliferation assays (FIGS. 36, 37). Pretreating IDHm ICC cells with AG-120 resulted in a substantial rescue from dasatinib-induced cytotoxicity, as shown in crystal violet staining, Annexin apoptosis assay and caspase-3 and PARP cleavage (FIGS. 38-40). Furthermore, AG-120 pretreatment (4 days) in all three IDHm ICC lines consistently showed a higher baseline level of pS6K/pS6 to IDH WT levels and partial rescue of dasatinib-mediated inhibition of pS6K/pS6 (FIG. 41). In addition to treatment with AG-120, the wild-type allele of IDH1 was knocked in (KI) into IDHm ICC cells. Restoration of the wild-type allele phenocopied treatment with AG-120 and also resulted in elevated pS6K/pS6 at baseline and a partial rescue from dasatinib-induced inhibition of pS6 (FIGS. 42, 43). Thus, both IDHm and pS6 levels act as a biomarker for dasatinib sensitivity in this experimental example.

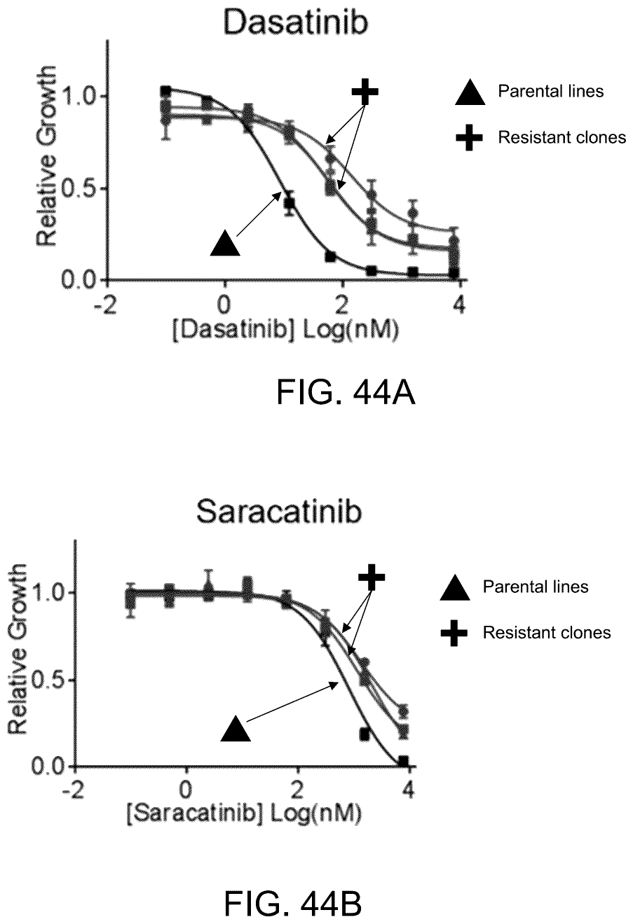

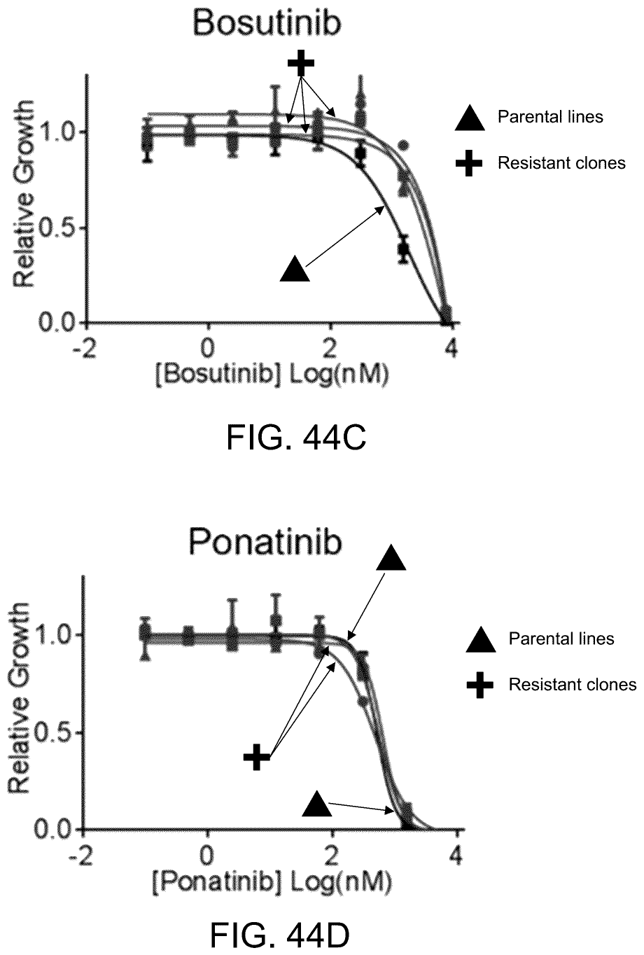

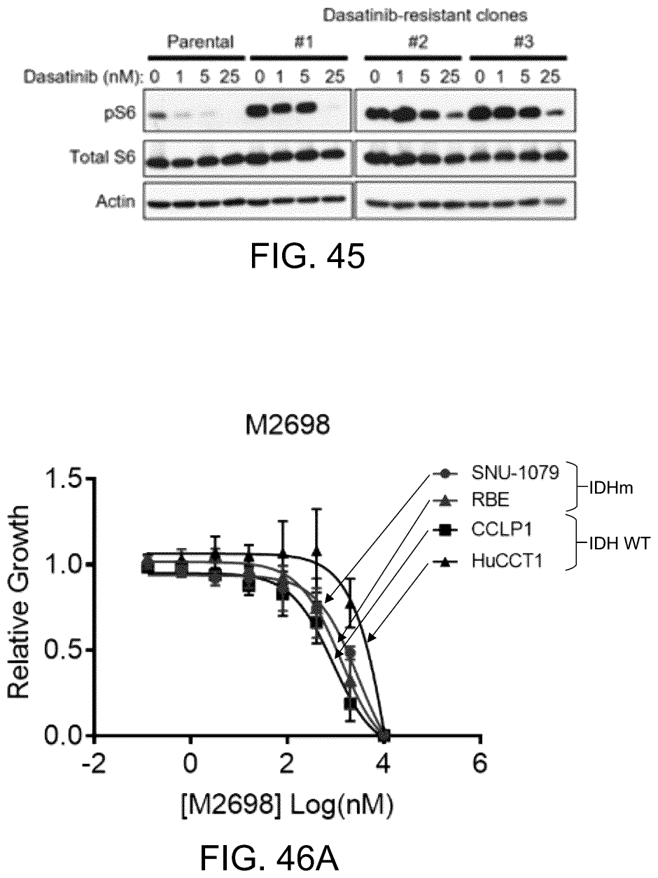

Next, dasatinib resistant clones were generated over several months by culturing in increasing concentrations of dasatinib. All three resistant clones have higher IC50 values compared to dasatinib and to other SRC inhibitors (FIG. 44A-D). These clones with acquired dasatinib resistance demonstrated elevated levels of pS6 at baseline and maintained higher pS6 levels with increasing doses of dasatinib (FIG. 45). The activation of PP2A through inhibition of SRC was not enough to keep pS6 levels reduced in this experimental example. The results disclosed herein show that additional inhibition of S6 upstream kinase activity is needed to synergize with dasatinib in IDHm cells to counter both intrinsic and acquired resistance through elevated pS6.

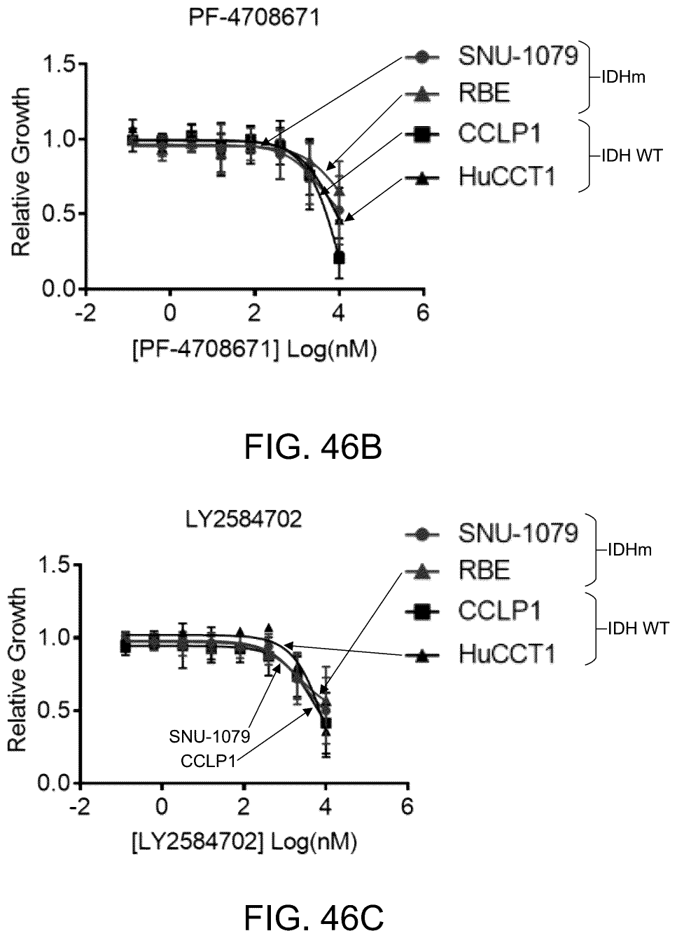

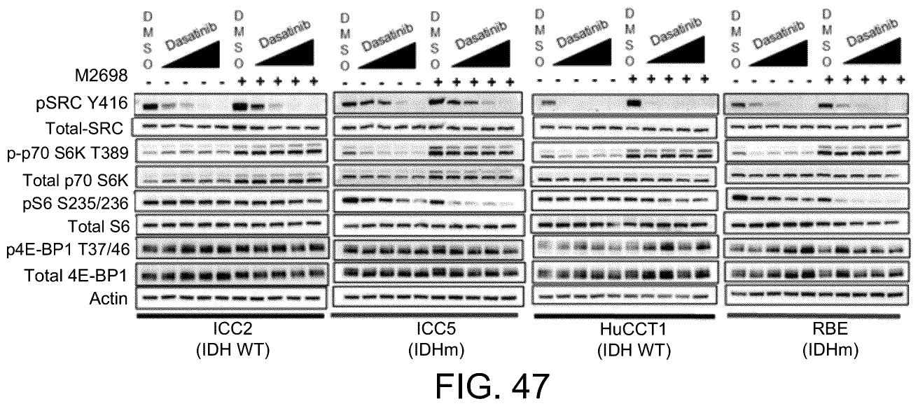

To test this, cells were treated with a variety of S6K inhibitors, such as PF-4708671 (Pearce et al., Biochem J 431, 245-255 (2010)), LY-2779964 (Tolcher et al., Eur J Cancer 50, 867-875 (2014)) and M2698 (Tsimberidou, et al., J Hematol Oncol 14, 127 (2021)). M2698, a potent, orally bioavailable, selective inhibitor against S6K, AKT1 and AKT3 had the most robust activity (FIGS. 46A-46C, Table 4).

TABLE 4
IC₅₀ values from FIGS. 46A-46C

| IC₅₀ (μM) | SNU-1079 | RBE | HuCCT1 | CCLP1 |
|---|---|---|---|---|
| M2698 | 2.91 | 1.26 | 25.14 | 0.86 |
| PF-4708671 | 4.28 | 10.07 | 8.76 | 14.58 |
| LY2584702 | 3.02 | 2.20 | 17.38 | 5.58 |

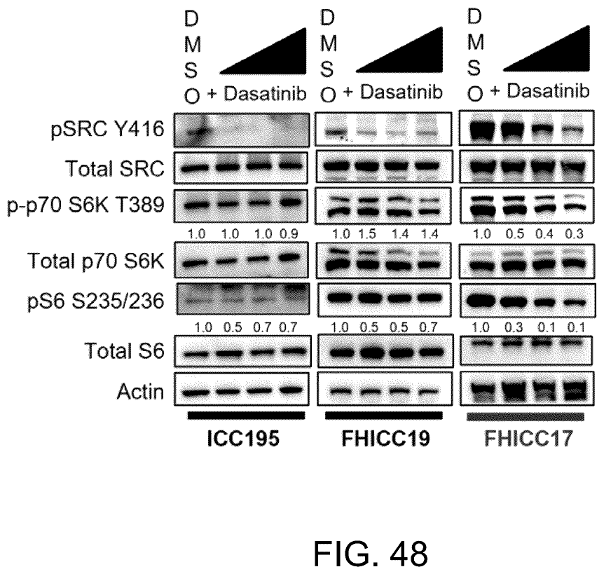

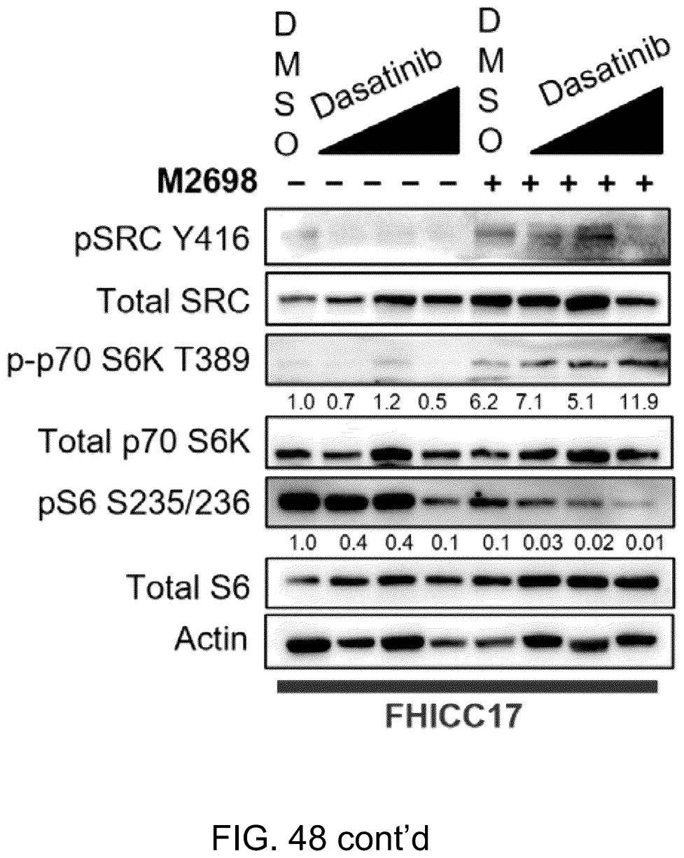

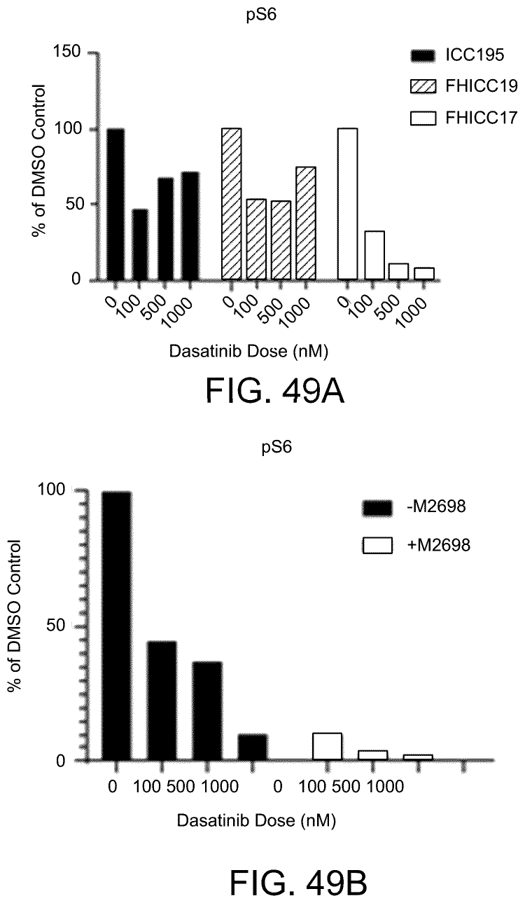

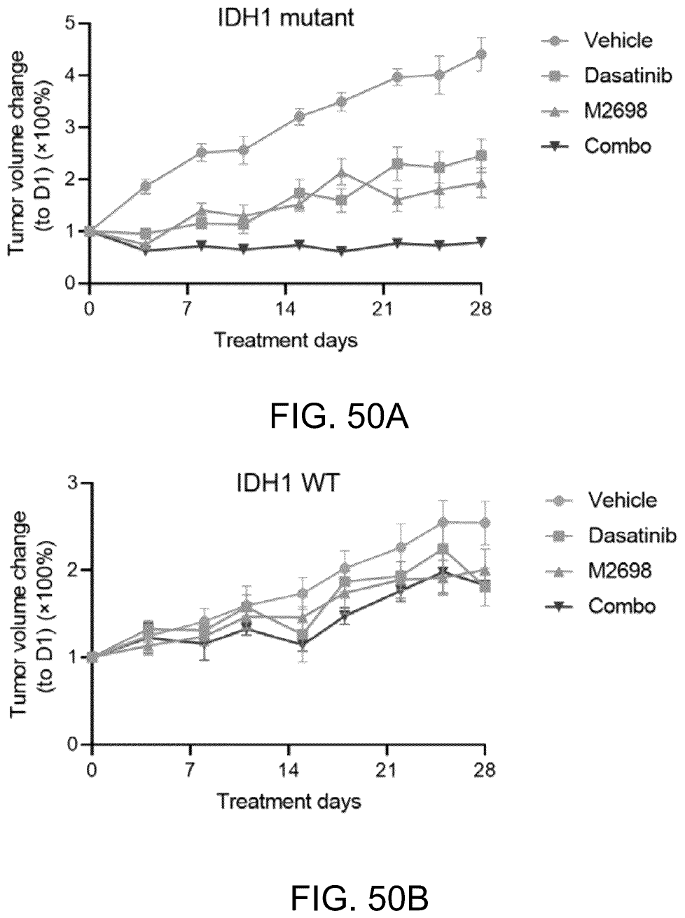

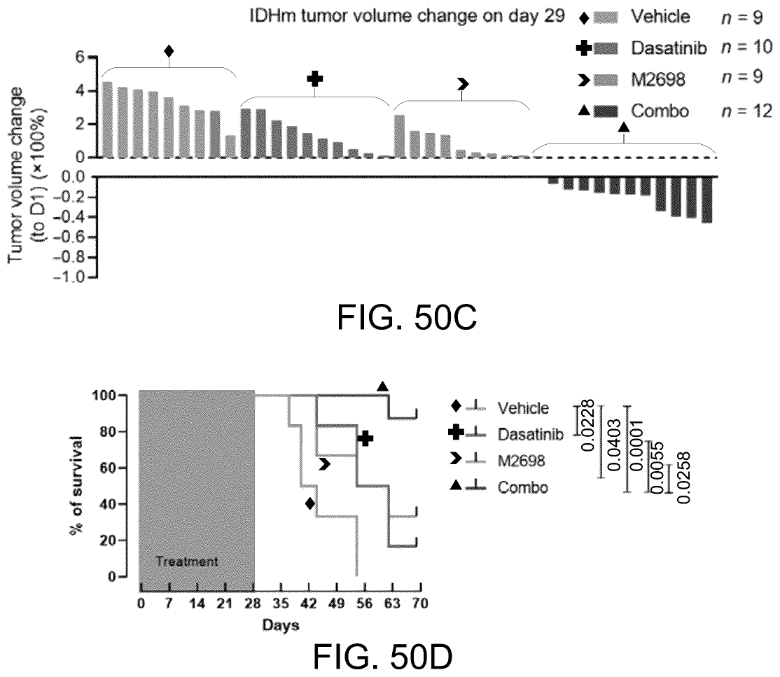

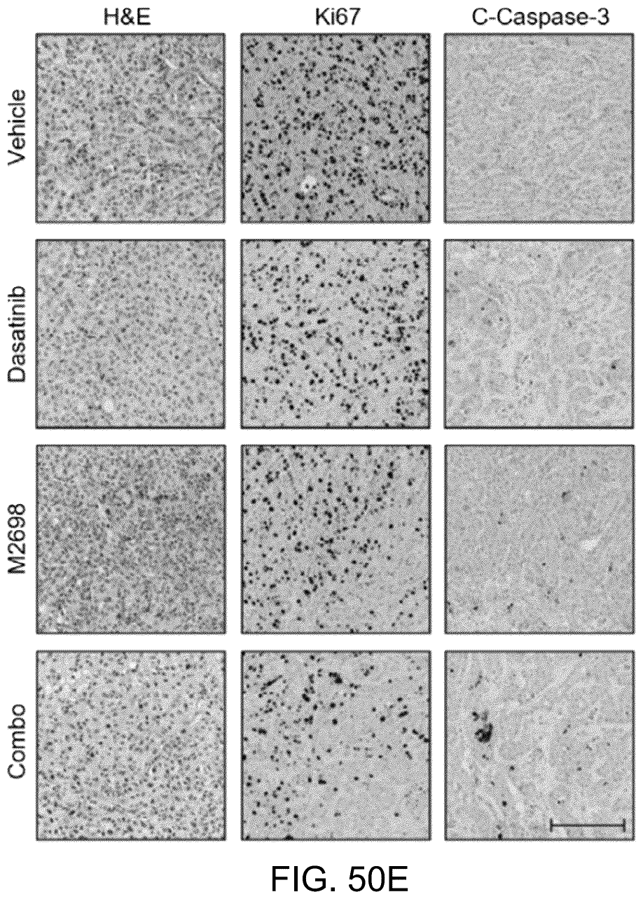

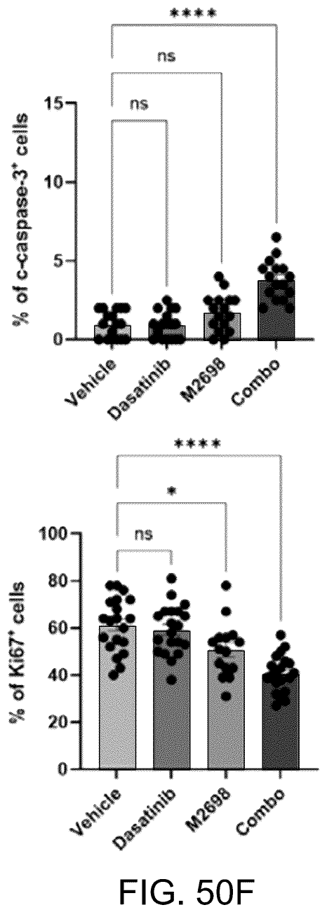

The combination of M2698 with dasatinib effectively reduced pS6 to undetectable levels in IDHm ICC cells while there was no change in IDH WT cells (FIG. 39). This was at clinically achievable doses of both dasatinib and M2698. Dasatinib was tested in combination with M2698 in IDHm and WT patient-derived organoids (PDOs), and it was found again that the combination substantially improved the reduction of pS6 levels in IDHm ICC organoids when compared to dasatinib alone, while there was no change in IDH WT PDOs (FIGS. 48, 49A, 49B). To study the antitumor efficacy of dasatinib and M2698 alone and in combination, in vivo drug trials were performed in IDHm and IDH WT ICC patient-derived xenograft models. Mice with established tumors were randomized into four arms—vehicle control, dasatinib only, M2698 only, and dasatinib+M2698 combination. PDX62 (IDH1 R132C) treated with a single treatment of either dasatinib or M2698 resulted in significant inhibitory effects relative to control. Excitingly, the combination of dasatinib+M2698 inhibited tumor growth to a much greater extent compared to either single drug alone (FIG. 50A). In contrast, the same single treatment and combination regimens did not cause an observable change in growth in ICC195 (IDH WT) PDX, (FIG. 50B). Dasatinib+M2698 resulted in tumor shrinkage in PDX62 (IDH1 R132C) at day 29, which was not seen in either single arm (FIG. 50C). Treatment with either agent resulted in prolonged survival, which was further improved in the combination arm (FIG. 50D). Immunohistochemistry (IHC) showed an increase of apoptosis and reduction of proliferation in the combination arm as measured by cleaved caspase-3 and Ki67 staining, respectively (FIGS. 50E, 50F).

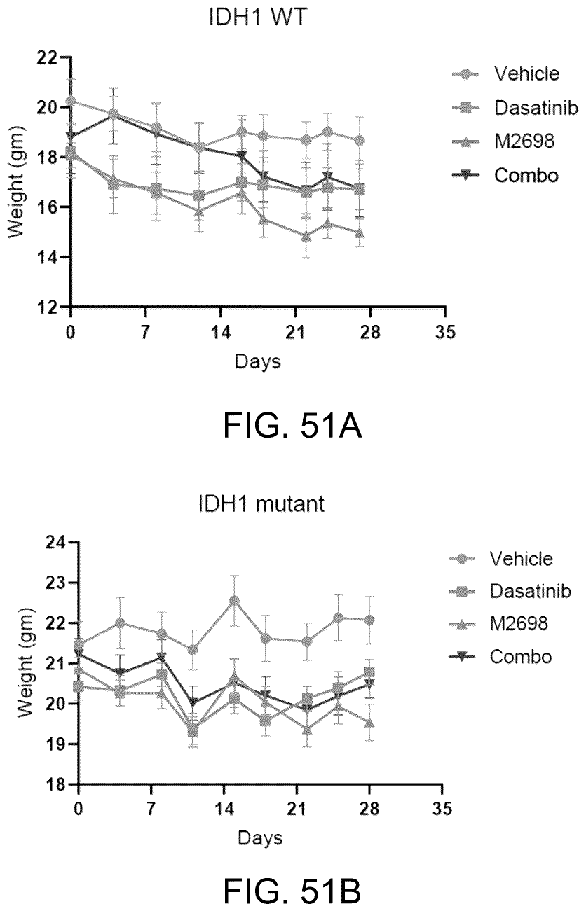

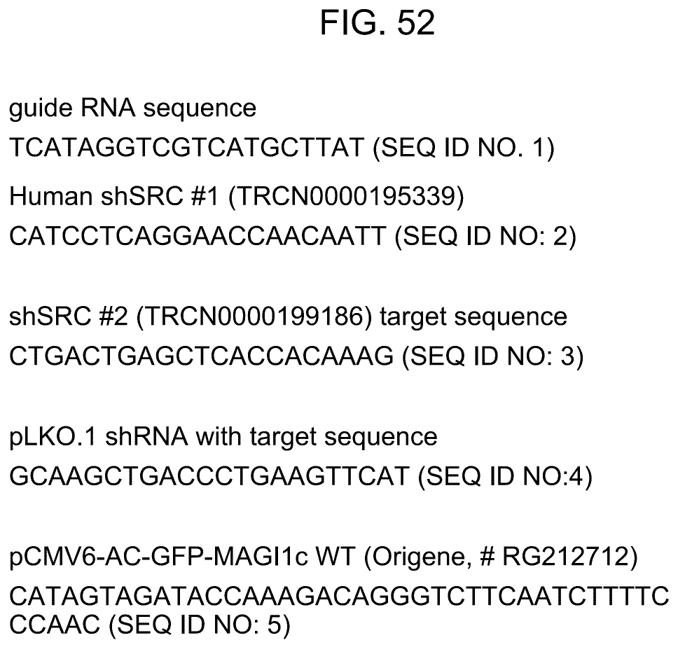

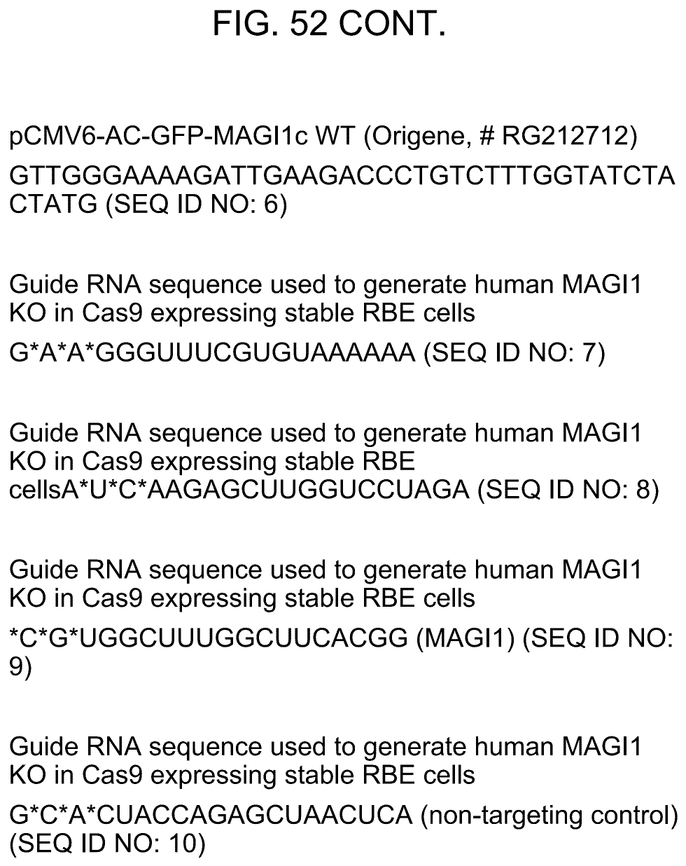

The dosages of dasatinib and M2698 are 30 mg/kg and 10 mg/kg, respectively, which are both below the single-agent effective doses used previously (Saha, et al., Cancer Discov 6, 727-739 (2016); Machl, et al., Am J Cancer Res 6, 806-818 (2016)). The combination treatment did not cause significant toxicity (FIGS. 51A, 51B). Taken together, the in vivo data from PDX models show that dasatinib in combination with S6K/AKT inhibitor M2698 presents a therapeutic option for IDHm ICC patients in this experimental example.

Discussion This experimental example shows that dasatinib-induced SRC inhibition leads to specific reduction of S6K/S6 signaling through de-phosphorylation of an adaptor protein, MAGI1, and activation of PP2A. Inhibiting SRC/S6K/S6 signaling reduced global protein translation and cell growth and eventually led to apoptosis. PP2A holoenzyme is a serine/threonine phosphatase that is composed of structural (A), regulatory (B) and catalytic (C) subunits and targets many substates implicated in oncogenic pathways including myc (Arnold and Sears, Cancer Metastasis Rev 27,147-158 (2008)), ERK, MEK (Abraham, et al., J Biol Chem 275, 22300-22304 (2000)), Akt (Ivaska, et al., Mol Cell Biol 22, 1352-1359 (2002)) and S6K (Peterson, et al., Proc Natl Acad Sci USA 96, 4438-4442 (1999); Westphal, et al., J Biol Chem 274, 687-692 (1999)). As a tumor suppressor, PP2A is frequently mutated or functionally inhibited in many common malignancies including breast, prostate, lungs, colon, melanoma, etc. Ongoing studies are aiming to simultaneously inhibit oncogenic kinases and activate PP2A to enhance anti-tumor activity. The current disclosure describes PP2A involvement in cholangiocarcinoma. The results described herein show that PP2A-mediated dephosphorylation of S6K is the key effector response upon SRC inhibition in IDHm ICC cells.

Dasatinib inhibits SRC equally in IDH WT and mutant cells but S6K and S6 phosphorylation are only reduced in the mutant counterparts. Although MAGI1 is expressed at a similar level in both IDH mutant and IDH WT cells, phosphorylated MAGI1 at Y373 is detected at a much lower level in WT than IDH mutant cells based on phosphoproteomics data (FIGS. 17A, 17B). The difference in MAGI1 baseline phosphorylation contributes to the differential activation of PP2A and subsequent sensitivity to dasatinib between IDH WT and mutant ICC cells.

Increasing evidence suggests that MAGI1 functions as a tumor suppressor (Zaric, et al., Oncogene 31, 48-59 (2012); Kozakai, et al., Int J Hematol 107, 337-344 (2018)). With multiple PDZ domains, MAGI1 can bring together various binding partners to facilitate signaling, an example of which is the membrane recruitment of PTEN (Zmajkovicova, et al., Mol Cell 50, 43-55 (2013)). Without being bound by theory, MAGI1 recruited activated PP2A and S6K to turn off survival signaling in an SRC-regulated manner. When IDHm cells are treated with dasatinib, SRC mediated phosphorylation is blocked, derepressing both MAGI1 and PP2A. This allows for SRC-MAGI1-PP2A binding. The now active PP2AC dephosphorylates S6K leading to inactivation of S6K/S6 signaling and cell death.

mTOR controls protein synthesis, at least in part, through direct phosphorylation of the tumor suppressor eukaryotic translation initiation factor 4E-BP1 and S6K. Evidence exists of an unexplained disconnect between S6K and 4E-BP1 phosphorylation, suggesting the presence of a mechanism to control their activity independent from mTORC1 activity and independent from each other. Treatment with rapamycin, a specific inhibitor of mTORC1, has shown differential regulation of 4E-BP1 and S6K, in a cell-specific manner. Rapamycin potently inhibited S6K activity but 4E-BP1 recovered phosphorylation over 6 hours despite mTOR inhibition (Choo, et al., Proc Natl Acad Sci USA 105, 17414-17419 (2008)). Similarly, primary B lymphocytes isolated from 4-wk-old Eμ-Myc mice show an unexpected and specific increase in mTORC1-dependent phosphorylation of 4E-BP1 while S6K on the contrary was not altered in this pretumor setting (Pourdehnad, et al., Proc Natl Acad Sci USA 110, 11988-11993 (2013)). The current disclosure describes PP2A-mediated dephosphorylation of S6K in a MAGI1-dependent manner revealing a new signaling complex that negatively regulates S6K/S6 activity. This discovery presents an explanation for the potential dissociation of 4E-BP1 and S6K phosphorylation.

There are similarities involving interactions among other viral and cellular proteins. Adenovirus type 9 (Ad9) E4-ORF1 and high-risk human papillomavirus (HPV) E6 proteins bind to the PDZ domains of MAGI1, resulting in MAGI1 being aberrantly sequestered in the cytoplasm by the Ad9 E4-ORF1 protein or being targeted for degradation by high-risk HPV E6 proteins. The authors of these studies have surmised that the tumorigenic potentials of these viral oncoproteins may depend, in part, on an ability to inhibit the function of MAGI1 in cells (Glaunsinger, et al., Oncogene 19, 5270-5280 (2000); Kranjec and Banks, J Virol 85, 1757-1764 (2011); Kranjec, et al., J Virol 88, 7155-7169 (2014); Araujo-Arcos, et al., Sci Rep 12, 1898 (2022)). Polyomavirus middle-T antigen (MT), SRC, and PP2A also form a similar signaling complex (Schaffhausen and Roberts, Virology 384, 304-316 (2009)). Like MAGI1, MT contains no intrinsic kinase activity and relies on recruitment of SRC to activate downstream oncogenic cell signaling. In an ordered sequence of interactions, MT binds to the core dimer of PP2A (Pallas, et al., Cell 60, 167-176 (1990); Walter, et al., Proc Natl Acad Sci USA 87, 2521-2525 (1990)) and then to a member of the SRC family of tyrosine kinases, usually pp60c-src (Courtneidge and Smith, Nature 303, 435-439 (1983)) or pp62c-yes (Kornbluth, et al., Nature 325, 171-173 (1987)). This activates the kinase activity of SRC, which phosphorylates tyrosines within MT. Three of these phosphotyrosines act as binding sites for the SH2 or PTB domains of PI3K (MT Y315) (Talmage, et al., Cell 59, 55-65 (1989)), ShcA (Y250) (Campbell, et al., Proc Natl Acad Sci USA 91, 6344-6348 (1994); Dilworth, et al., Nature 367, 87-90 (1994)), and phospholipase C-y1 ([PLC-γ1]Y322) (Su, et al., J Biol Chem 270, 12331-12334 (1995)). As a consequence of their interaction with MT, each of these polypeptides is, in turn, tyrosine phosphorylated, which activates PI3K- and PLC-γ1—dependent signaling pathways and creates a binding site on ShcA for Grb2 (Dilworth, supra). The guanine nucleotide exchange factor Sos1 and the adapter molecule Gab1 (Nicholson, et al., EMBO J 20, 6337-6346 (2001)) are brought into the MT complex through their interactions with Grb2, thereby activating Ras and the ERK kinase cascade (Li, et al., Nature 363, 85-88 (1993); Rozakis-Adcock, et al., Nature 363, 83-85 (1993)). However, unlike MT complex, SRC-MAGI1-PP2A appears to be growth suppressive through inactivation of S6K/S6 signaling. Further assessment of the similarities and differences between MAGI1 and MT may provide new insights in exploring the biochemical regulation of this SRC-MAGI1-PP2A survival signaling complex in ICC and other cancers.

Inhibition of 2-HG by treatment with AG-120 and IDH1 WT KI could partially reverse dasatinib hypersensitivity and S6K/S6 signaling, suggesting that 2-HG promotes reliance on SRC in ICC cells. One possible mechanism is through acting on one or more of the 70 aKG-dependent dioxygenase family enzymes that have diverse cellular functions, including epigenetic modifications, DNA damage repair, collagen synthesis, and hypoxia response. A recent report suggested that 2-HG can activate mTOR signaling through inhibition of histone demethylase lysine demethylase 4A, one of the αKG dioxygenase family enzymes (Carbonneau, et al., Cell 160, 324-338 (2015)). Experiments performed in endogenous IDH1 R132C fibrosarcoma line HT1080, in which the authors reported that inhibiting IDHm by earlier generation of IDH1m-specific inhibitor AGI-5198 resulted in a reduction of mTOR signaling, showed an opposite effect to what was observed in this Example (P. R. Nicholson, supra). This may indicate a difference in tissue specificity in terms of the implicated dioxygenase enzymes targeted by 2-HG. The data presented in this Example may have important clinical implications, because treating ICC with ivosidenib could make ICC cells more resistant to dasatinib; therefore, combination or sequential applications of dasatinib and ivosidenib in patients with ICC may have to be avoided.

This Experimental Example shows that pS6 abundance predicts intrinsic and extrinsic resistance to dasatinib in multiple ICC cell line models, clinical samples, and PDOs. Targeting pS6 is of critical importance in overcoming dasatinib resistance, and combination treatment with dasatinib and M2698 was demonstrated to effectively suppress pS6 signaling. Inhibitors targeting mTOR and PI3K have shown limited efficacy because inhibition of a single node in the mTOR/PI3K pathway can lead to compensatory activation, usually of AKT, via release of a negative feedback loop (LoRusso, J. Clin. Oncol. 34, 3803-3815 (2016); O'Reilly, et al., Cancer Res. 66, 1500-1508 (2006)). M2698 has the potential to block the AKT compensatory feedback loop while avoiding the adverse effects of pan-AKT inhibition (ipatasertib, capivasertib, GSK690693, and MK-2206), including those associated with AKT2 inhibition (Wang, et al., Br. J. Cancer 117, 159-163 (2017)). M2698 has been well tolerated in a phase 1 clinical trial (Tsimberidou, supra), indicating that M2698 and dasatinib provides a combination for patients with IDHm ICC.

The current disclosure identifies pS6 as a biomarker for SRC inhibitor (ex. dasatinib) sensitivity and describe a combination therapy of an SRC inhibitor and an S6K/AKT inhibitor to reduce pS6 levels and improve cell growth inhibition over SRC inhibitor treatment alone particularly in IDHm ICC

(vi) Closing Paragraphs. Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

The terms “inhibiting” and “reducing” or any variation of these terms, when used in the claims and/or the specification includes any measurable decrease or complete inhibition to achieve a desired result. The terms “prevention” and “preventing” refer to the expectation that something can be kept from happening to some extent or that the severity, duration, or extent of the condition or disease can be alleviated or reduced. It is contemplated that the terms “treating” or “preventing” in the context of a condition or disease refers to any reduction or inhibition of the disease or condition. In specific embodiments, the disease or condition is a hyperproliferative disease or condition. In certain other cases, embodiments pertain to cancer or tumors. In specific embodiments, the cancer is breast, ovarian, prostate or colon cancer.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds, nucleic acid, and amino acids sequences referenced herein that are available in a public database, the information in the database entry is incorporated herein by reference as of the date of an application in the priority chain in which the database identifier for that compound or sequence was first included in the text.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood et al., Oxford University Press, Oxford, 2006).

What is claimed is:

1. A method of treating a subject having a condition associated with IDH1/2 mutation(s), the method comprising administering to the subject
a therapeutically effective amount of an SRC inhibitor; and
a therapeutically effective amount of an S6K/AKT inhibitor,
thereby treating the subject having the condition associated with IDH1/2 mutation(s).

2. The method of claim 1, wherein the SRC inhibitor comprises dasatinib, saracatinib, bosutinib, NXP900, KX01, KX2-391, PP1, or PP2.

3. The method of claim 1, wherein the SRC inhibitor comprises dasatinib or saracatinib.

4. The method of claim 1, wherein the S6K/AKT inhibitor comprises M2698, pyrazolopyrimidines, LY2780301, LY2584702, GNE-477, paxalisib, pyrvinium pamoate, PF-4708671, or MSC2363318A.

5. The method of claim 1, wherein the SRC inhibitor comprises dasatinib and the S6K/AKT inhibitor comprises M2698.

6. The method of claim 1, wherein the condition associated with IDH1/2 mutation(s) comprises cholangiocarcinoma, oligodendroglioma, astrocytoma, leukemia, adenocarcinoma, glioma, melanoma, oligoastrocytoma, breast carcinoma, or myelodysplastic syndrome.

7. The method of claim 6, wherein the cholangiocarcinoma comprises intrahepatic cholangiocarcinoma.

8. The method of claim 1, wherein the administering is intravenous, intradermal, intraarterial, intranodal, intravesicular, intrathecal, intraperitoneal, intraparenteral, intranasal, intralesional, intramuscular, oral, intrapulmonary, subcutaneous, or sublingual.

9. The method of claim 1, wherein the SRC inhibitor and the S6K/AKT inhibitor are administered together.

10. The method of claim 1, further comprising assessing a phosphorylation level of S6 in a sample derived from the subject.

11. The method of claim 1, wherein a phosphorylation level of S6 in a sample derived from the subject exceeded a threshold.

12. The method of claim 1, further comprising identifying IDH1 and/or IDH2 mutation(s) in a sample derived from the subject.

13. The method of claim 1, wherein the subject has IDH1/2 mutation(s) comprising an IDH1 mutation at residue 132.

14. The method of claim 1, wherein the subject has IDH1/2 mutation(s) comprising an IDH2 mutation at residue 140.

15. The method of claim 1, wherein the subject has IDH1/2 mutation(s) comprising an IDH2 mutation at residue 172.

16. The method of claim 1, wherein the subject has IDH1/2 mutation(s) comprising an IDH1 mutation at residue 132 and an IDH2 mutation at residue 140.

17. The method of claim 1, wherein the subject has IDH1/2 mutation(s) comprising an IDH1 mutation at residue 132 and an IDH2 mutation at residue 172.

18. The method of claim 1, wherein the subject has IDH1/2 mutation(s) comprising an IDH2 mutation at residue 140 and an IDH2 mutation at residue 172.

19. The method of claim 1, further comprising diagnosing the subject with the condition associated with IDH1/2 mutation(s).

20. A method for evaluating a subject having a condition associated with IDH1/2 mutation(s) for enrollment in a clinical trial, the method comprising
assessing a phosphorylation level of S6 in a sample derived from the subject; and
enrolling the subject in the clinical trial if the phosphorylation level meets or exceeds a threshold.

21. The method of claim 20, wherein the condition associated with IDH1/2 mutation(s) comprises cholangiocarcinoma, oligodendroglioma, astrocytoma, leukemia, adenocarcinoma, glioma, melanoma, oligoastrocytoma, breast carcinoma, or myelodysplastic syndrome.

22. The method of claim 21, wherein the cholangiocarcinoma comprises intrahepatic cholangiocarcinoma.

23. The method of claim 20, wherein the clinical trial comprises a protocol directing administration of an SRC inhibitor and an S6K/AKT inhibitor.

24. The method of claim 23, wherein the SRC inhibitor comprises dasatinib, saracatinib, bosutinib, NXP900, KX01, KX2-391, PP1, or PP2.

25. The method of claim 23, wherein the SRC inhibitor comprises dasatinib or saracatinib.

26. The method of claim 23, wherein the S6K/AKT inhibitor comprises M2698, pyrazolopyrimidines, LY2780301, LY2584702, GNE-477, paxalisib, pyrvinium pamoate, PF-4708671, or MSC2363318A.

27. The method of claim 23, wherein the SRC inhibitor comprises dasatinib and the S6K/AKT inhibitor comprises M2698.

28. The method of claim 20, wherein the subject has IDH1/2 mutation(s) comprising an IDH1 mutation at residue 132.

29. The method of claim 20, wherein the subject has IDH1/2 mutation(s) comprising an IDH2 mutation at residue 140.

30. The method of claim 20, wherein the subject has IDH1/2 mutation(s) comprising an IDH2 mutation at residue 172.

31. The method of claim 20, wherein the subject has IDH1/2 mutation(s) comprising an IDH1 mutation at residue 132 and an IDH2 mutation at residue 140.

32. The method of claim 20, wherein the subject has IDH1/2 mutation(s) comprising an IDH1 mutation at residue 132 and an IDH2 mutation at residue 172.

33. The method of claim 20, wherein the subject has IDH1/2 mutation(s) comprising an IDH2 mutation at residue 140 and an IDH2 mutation at residue 172.

34. A composition comprising an SRC inhibitor, an S6K/AKT inhibitor, and a pharmaceutically acceptable carrier.

35. The composition of claim 34, wherein the SRC inhibitor comprises dasatinib, saracatinib, bosutinib, NXP900, KX01, KX2-391, PP1, or PP2.

36. The composition of claim 34, wherein the SRC inhibitor comprises dasatinib or saracatinib.

37. The composition of claim 34, wherein the S6K/AKT inhibitor comprises M2698, pyrazolopyrimidines, LY2780301, LY2584702, GNE-477, paxalisib, pyrvinium pamoate, PF-4708671, or MSC2363318A.

38. The composition of claim 34, wherein the SRC inhibitor comprises dasatinib and the S6K/AKT inhibitor comprises M2698.

39. A kit comprising an SRC inhibitor and an S6K/AKT inhibitor.

40. The kit of claim 39, further comprising a pharmaceutically acceptable carrier.

41. The kit of claim 39, wherein the SRC inhibitor comprises dasatinib, saracatinib, bosutinib, NXP900, KX01, KX2-391, PP1, or PP2.

42. The kit of claim 39, wherein the SRC inhibitor comprises dasatinib or saracatinib.

43. The kit of claim 39, wherein the S6K/AKT inhibitor comprises M2698, pyrazolopyrimidines, LY2780301, LY2584702, GNE-477, paxalisib, pyrvinium pamoate, PF-4708671, or MSC2363318A.

44. The kit of claim 39, wherein the SRC inhibitor comprises dasatinib and the S6K/AKT inhibitor comprises M2698.

45. The kit of claim 39, further comprising instructions to administer the SRC inhibitor and the S6K/AKT inhibitor to a subject having a condition associated with IDH1/2 mutation(s).

46. The kit of claim 45, wherein the condition associated with IDH1/2 mutation(s) comprises cholangiocarcinoma, oligodendroglioma, astrocytoma, leukemia, adenocarcinoma, glioma, melanoma, oligoastrocytoma, breast carcinoma, or myelodysplastic syndrome.

47. The kit of claim 46, wherein the cholangiocarcinoma comprises intrahepatic cholangiocarcinoma.

48. The kit of claim 39, further comprising instructions to administer the SRC inhibitor and the S6K/AKT inhibitor to a subject having an S6 phosphorylation level that exceeds a threshold.

49. A method of treating a subject in need thereof, comprising:
administering to the subject in need:
a therapeutically effective amount of an SRC inhibitor; and
a therapeutically effective amount of an S6K/AKT inhibitor.

50. The method of claim 49, wherein the SRC inhibitor comprises one or more of: dasatinib, saracatinib, bosutinib, NXP900, KX01, KX2-391, PP1, or PP2.

51. The method of claim 49, wherein the SRC inhibitor comprises dasatinib or saracatinib.

52. The method of claim 49, wherein the S6K/AKT inhibitor comprises one or more of: M2698, pyrazolopyrimidines, LY2780301, LY2584702, GNE-477, paxalisib, pyrvinium pamoate, PF-4708671, or MSC2363318A.

53. The method of claim 49, wherein the therapeutically effective amount provides a therapeutic treatment against a condition associated with IDH1/2 mutation(s).

54. The method of claim 53, wherein the condition associated with IDH1/2 mutation(s) comprises at least one of: a cholangiocarcinoma, an oligodendroglioma, an astrocytoma, a leukemia, an adenocarcinoma, a glioma, a melanoma, an oligoastrocytoma, a breast carcinoma, or a myelodysplastic syndrome.

55. The method of claim 53, wherein the condition associated with IDH1/2 mutation(s) comprises intrahepatic cholangiocarcinoma.

56. The method of claim 49, wherein the therapeutically effective amount of the SRC inhibitor comprises a dosage of 30 mg/kg.

57. The method of claim 49, wherein the therapeutically effective amount of the S6K/AKT inhibitor comprises a dosage of 10 mg/kg.

58. The method of claim 49, wherein the administering is through intravenous, intradermal, intraarterial, intranodal, intravesicular, intrathecal, intraperitoneal, intraparenteral, intranasal, intralesional, intramuscular, oral, intrapulmonary, subcutaneous, or sublingual administering.

59. The method of claim 49, wherein the therapeutically effective amount of the SRC inhibitor and the therapeutically effective amount of the S6K/AKT inhibitor are administered together.

60. The method of claim 49, wherein the therapeutically effective amount of the SRC inhibitor is administered within a clinically relevant time window of the therapeutically effective amount of an S6K/AKT inhibitor.

61. The method of claim 49, wherein the subject in need thereof has a level of phosphorylated S6 (pS6) 5% to 300% higher than a reference level, wherein the reference level comprises a pS6 level obtained from:
a biological sample of an individual who does not have cancer,
the subject at an earlier point in time, or
an individual with cancer without an IDH1/2 mutation.

62. The method of claim 61, wherein the level of pS6 in the subject comprises a range of 10 to 75% higher than the reference level.

63. The method of claim 49, further comprising identifying IDH1 and/or IDH2 mutation(s) in the subject.

64. The method of claim 63, wherein the identifying identifies IDH1/2 mutation(s) comprising an IDH1 mutation at residue 132.

65. The method of claim 63, wherein the identifying identifies IDH1/2 mutation(s) comprising an IDH2 mutation at residue 140.

66. The method of claim 63, wherein the identifying identifies IDH1/2 mutation(s) comprising an IDH2 mutation at residue 172.

67. The method of claim 63, wherein the identifying identifies IDH1/2 mutation(s) comprising an IDH1 mutation at residue 132 and an IDH2 mutation at residue 140.

68. The method of claim 63, wherein the identifying identifies IDH1/2 mutation(s) comprising an IDH1 mutation at residue 132 and an IDH2 mutation at residue 172.

69. The method of claim 63, wherein the identifying identifies IDH1/2 mutation(s) comprising an IDH2 mutation at residue 140 and an IDH2 mutation at residue 172.

70. The method of claim 63, wherein identifying one or more IDH1 and/or IDH2 mutation(s) in the subject comprises performing on a sample comprising cells of the subject one or more of: direct nucleic acid sequencing, hybridization methods, restriction enzyme digestion, polymerase chain reaction (PCT) amplification, or protein detection.

71. A method of treating a subject in need thereof, comprising:
measuring a level of phosphorylated S6 (pS6) in a sample from the subject;
determining if the level of pS6 in the subject is higher than a reference pS6 level; and
when the level of pS6 in the subject is higher than the reference pS6 level administering to the subject:
a therapeutically effective amount of an SRC inhibitor; and
a therapeutically effective amount of an S6K/AKT inhibitor.

72. The method of claim 71, wherein the level of pS6 in the subject is determined via western blot, antibody detection, a kinase activity assay, flow cytometry, immunocytochemistry, immunohistochemistry, mass spectrometry, or multi-analyte profiling.

73. The method of claim 71, wherein the level of pS6 in the subject comprises a range of 5 to 300% higher than a reference level, wherein the reference level comprises a pS6 level obtained from:
a biological sample of an individual who does not have cancer,
the subject at an earlier point in time, or
an individual with cancer without an IDH1/2 mutation.

74. The method of claim 71, wherein the level of pS6 (pS6) in the subject comprises a range of 10 to 75% higher than the reference level.

75. A composition, comprising:
an SRC inhibitor; and
an S6K/AKT inhibitor.

76. The composition of claim 75, wherein the SRC inhibitor comprises dasatinib, saracatinib, bosutinib, NXP900, KX01, KX2-391, PP1, or PP2.

77. The composition of claim 75, wherein the SRC inhibitor comprises dasatinib or saracatinib.

78. The composition of claim 75, wherein the S6K/AKT inhibitor comprises M2698, pyrazolopyrimidines, LY2780301, LY2584702, GNE-477, paxalisib, pyrvinium pamoate, PF-4708671, or MSC2363318A.

79. The composition of claim 75, further comprising a pharmaceutically acceptable carrier.

80. The composition of claim 75, wherein the SRC inhibitor is formulated with a first pharmaceutically acceptable carrier and wherein the S6K/AKT inhibitor is formulated with a second pharmaceutically acceptable carrier.


SEQUENCE LISTING (10 sequences)

SEQ ID NO: 1 - DNA, 20 bp, synthetic construct
  source 1..20: mol_type = other DNA
tcataggtcgtcatgcttat

SEQ ID NO: 2 - DNA, 21 bp, Homo sapiens
  source 1..21: mol_type = other DNA
catcctcaggaaccaacaatt

SEQ ID NO: 3 - DNA, 21 bp, Homo sapiens
  source 1..21: mol_type = other DNA
ctgactgagctcaccacaaag

SEQ ID NO: 4 - DNA, 22 bp, synthetic construct
  source 1..22: mol_type = other DNA
gcaagctgaccctgaagttcat

SEQ ID NO: 5 - DNA, 42 bp, synthetic construct
  source 1..42: mol_type = other DNA
catagtagataccaaagacagggtcttcaatcttttcccaac

SEQ ID NO: 6 - DNA, 42 bp, synthetic construct
  source 1..42: mol_type = other DNA
gttgggaaaagattgaagaccctgtctttggtatctactatg

SEQ ID NO: 7 - RNA, 20 nt, synthetic construct
  source 1..20: mol_type = other RNA
  modified_base 1: mod_base = gm
  modified_base 2: mod_base = m2a
  modified_base 3: mod_base = m2a
  modified_base 1..4: mod_base = OTHER; note = 3 prime-phosphorothioate internucleotide linkages between positions 1 and 2, positions 2 and 3, and positions 3 and 4
gaagggtttcgtgtaaaaaa

SEQ ID NO: 8 - RNA, 20 nt, synthetic construct
  source 1..20: mol_type = other RNA
  modified_base 1: mod_base = m2a
  modified_base 2: mod_base = um
  modified_base 3: mod_base = cm
  modified_base 1..4: mod_base = OTHER; note = 3 prime-phosphorothioate internucleotide linkages between positions 1 and 2, positions 2 and 3, and positions 3 and 4.
atcaagagcttggtcctaga

SEQ ID NO: 9 - RNA, 20 nt, synthetic construct
  source 1..20: mol_type = other RNA
  modified_base 1: mod_base = um
  modified_base 2: mod_base = cm
  modified_base 3: mod_base = gm
  modified_base 1..4: mod_base = OTHER; note = 3 prime-phosphorothioate internucleotide linkages between positions 1 and 2, positions 2 and 3, and positions 3 and 4.
tcgtggctttggcttcacgg

SEQ ID NO: 10 - RNA, 20 nt, synthetic construct
  source 1..20: mol_type = other RNA
  modified_base 1: mod_base = gm
  modified_base 2: mod_base = cm
  modified_base 3: mod_base = m2a
  modified_base 1..4: mod_base = OTHER; note = 3 prime-phosphorothioate internucleotide linkages between positions 1 and 2, positions 2 and 3, and positions 3 and 4.
gcactaccagagctaactca